SEQUENCE LISTING (2 sequences)

SEQ ID NO: 1 - protein, 23 aa, Artificial Sequence
  Synthetic peptide
QEPGGLVVPPTDAPVSPTTLYVE

SEQ ID NO: 2 - protein, 24 aa, Artificial Sequence
  Synthetic peptide
QLPTGVLGEPSDAVPTVYEVTPPG

(19) United States
(12) Patent Application Publication
(10) Pub. No.: US 2015/0160214 A1
(43) Pub. Date: Jun. 11, 2015

(54) Quantitation of Cellular Adhesion Dynamics Across Immobilized Receptors Under Rheological Shear Flow

(71) Applicant: Mayo Foundation for Medical Education and Research, Rochester, MN (US)

(72) Inventor: Matthew T. Auton, Rochester, MN (US)

(21) Appl. No.: 14/558,328

(22) Filed: Dec. 2, 2014

Related U.S. Application Data
(60) Provisional application No. 61/912,354, filed on Dec. 5, 2013.

(51) Int. Cl. G01N 33/569; G01N 33/86; G01N 33/50

(52) U.S. Cl. CPC G01N 33/56966; G01N 33/5032; G01N 33/86; G01N 2800/224; G01N 2500/10

(57) ABSTRACT

The present invention includes an apparatus and methods for measuring cell or platelet adhesion comprising: a rheological shear flow surface coated with an agent that provides cell or platelet adhesion; a detector to track the transit of cells or platelets on the surface under shear flow; and a processor that calculates both a pause time and a roll time of platelets, wherein the processor determines a median and a mean pause time and a median and a mean roll time of cells or platelets and compares them to cells or platelets having at least one of: no cell adhesion dysfunction, a mild adhesion dysfunction, or a severe adhesion dysfunction, between a cell surface adhesion molecule on the cell or platelet and the agent, wherein the processor provides a real-time, quantitative measurement of a dynamic range of cell or platelet adhesion.

### CROSS-REFERENCE TO RELATED APPLICATIONS

This application claims priority to U.S. Provisional Patent Application Ser. No. 61/912,354, filed Dec. 5, 2013, the entire contents of which are incorporated herein by reference.

### STATEMENT OF FEDERALLY FUNDED RESEARCH

This invention was made with U.S. government support by the National Heart Lung and Blood Institute of the National Institutes of Health, Grant No. HL109109. The government has certain rights in this invention.

### TECHNICAL FIELD OF THE INVENTION

The present invention relates in general to the field of disease conditions that involve cell adhesion, and more particularly, to methods and devices for measuring and directing treatment of conditions based on cellular adhesion dynamics across immobilized receptors under rheological shear flow.

### INCORPORATION-BY-REFERENCE OF MATERIALS FILED ON COMPACT DISC

None.

### BACKGROUND OF THE INVENTION

Without limiting the scope of the invention, its background is described in connection with cell adhesion.

Presently commercially available equipment to perform adhesion assay and commercially available software to track the movement of platelets during the video time-frame provide no means to statistically quantify the physics of instantaneous cell movements for many cells over a long time-frame. Current methods published in the literature quantify velocities of cell movement as an average distance travelled over a specified amount of time. Furthermore, these methods restrict analysis to the movement of only one cell at a time, a user time intensive process of analysis. Another disadvantage of this approach is that it cannot quantify instantaneous movements because only the start and end positions are considered.

As a result of these limitations, existing guidelines for the use of platelet adhesion laboratory tests exclude adhesion studies except for cases of high adherence or no adherence. For example, the Guidelines for the Laboratory Investigation of Heritable Disorders or Platelet Function, from the British Committee for Standards in Haematology, published August 2011 (Writing group: Paul Harrison et al.) (hereinafter the Guidelines), found, with regard to shear stress systems, that “[i]t is not currently possible to accurately determine the sensitivity of the PFA-100® for most mild, heritable platelet function defects since most reported studies comprise small patient numbers, with varying mixtures of these defects.” (Guidelines, pp. 10-11.) Existing devices were found to exhibit “poor sensitivity for mild platelet defects in a small number of prospective studies in patients with an unequivocal personal and family history of mucocutaneous bleeding.” Id. at 11. In conclusion, the Guidelines state that existing technology “ . . . provides an optional screening test, but this must be interpreted with caution and in the context of the clinical background, as the test is not diagnostic or sensitive for mild platelet disorders (1B).”

Another method for detecting changes in binding proteins, specifically, von Willebrand Factor (vWF) is taught in U.S. Patent Application Serial No. 2009/0202429, filed by Diacovo et al., entitled, Methods For Testing Anti-Thrombotic Agents. Briefly, this application provides a transgenic non-human animal expressing vWF A1 protein containing at least one mutation selected from the group consisting of: 1263P>S, 1269N>D, 1274K>R, 1287M>R, 1302G>D, 1308H>R, 1313R>W, 1314I>V, 1326R>H, 1329L>I, 1330E>G, 1333A>D, 1344T>A, 1347I>V, 1350T>A, 1370G>S, 1379H>R, 1381T>A, 1385T>M, 1391P>Q, 1394A>S, 1397L>F, 1421S>N, 1439L>V, 1442G>S, 1449R>Q, 1466A>P, 1469Q>L, 1472Q>H, 1473V>M, 1475H>Q, 1479S>G, and any combination thereof.

Another method is taught in U.S. Patent Application Publication No. 2013/0109012, filed by Sniadecki et al., entitled, Device To Expose Cells to Fluid Shear Forces and Associated Systems And Methods. Briefly, these applicants are said to disclose devices to expose cells to fluid shear forces, and associated systems and methods. In particular, several embodiments are directed toward devices to expose cells to fluid shear forces in order to measure changes in internal cell forces. A fluidic device includes a flow unit configured to induce fluid flow through the device, and may further include a fluid channel configured to accept a biological sample dispersed on an array of flexible structures. The flow unit can be configured to induce disturbed and/or laminar flow in the fluid channel. The device can further include optical or magnetic detection means configured to measure a degree of deflection of one or more flexible structures in the array.

Finally, U.S. Pat. No. 8,163,151, issued to Gabriel, entitled Assays for detection of von Willebrand Factor (vWF) multimers and for degradation of vWF by agents such as Adamts13 and related methods. Briefly, the patent application teaches methods of analyzing the electrophoretic mobility distribution of von Willebrand Factor (vWF) multimers include providing a sample medium comprising a plurality vWF multimers. The vWF multimers are electrophoretically separated by electrophoretic mobility in the sample medium by subjecting the sample medium to an electric field to provide separated vWF multimers. The separated vWF multimers in the sample medium are exposed to a light source to produce scattered light, which is detected, and the electrophoretic mobility distribution of the separated vWF multimers is determined from the detected scattered light.

### SUMMARY OF THE INVENTION

The present invention includes an apparatus and method for analyzing the movement of hundreds to thousands of cells or platelets resulting in distance travelled, instantaneous velocities, accelerations, and pause (residence) times. Statistical distributions of these physical properties of cell or platelet adhesion to a surface are obtained, which may be used to identify a disease involving cell or platelet adhesion, for evaluating candidate drugs that affect cell or platelet adhesion, and for taking real-time quantitative measurements of a dynamic range of cell or platelet function to select a treatment.

In one embodiment, the present invention includes an apparatus for measuring cell or platelet adhesion comprising: a rheological shear flow surface coated with an agent that provides cell or platelet adhesion; a detector to track the transit of cells or platelets on the surface under shear flow; and a processor that calculates both a pause time and a roll time of platelets, wherein the processor determines a median and a mean pause time and a median and a mean roll time of cells or platelets and compares them to cells or platelets having at least one of: no cell adhesion dysfunction, a mild adhesion dysfunction, or a severe adhesion dysfunction, between a cell surface adhesion molecule on the cell or platelet and the agent, wherein the processor provides a real-time, quantitative measurement of a dynamic range of cell or platelet adhesion. In one aspect, the cell surface adhesion molecule is von Willebrand Factor, an ICAM, a VCAM, a Lectin, an Integrin, a collagen, a fibrinogen, a subendothelial membrane protein, glycoprotein Ib(β), glycoproteinIb(α), glycoprotein Ib-IX-V complex, glycoprotein IIbIIIa, glyprotein VI, major histocompatibility complex, integrin α2 β1, or immunoglobin. In another aspect, the cell surface adhesion molecule is von Willebrand Factor protein comprising one or more point mutations in the A1, A2, A3, B, or C domains. In another aspect, the surface is coated with a mutant human von Willebrand factor protein, collagen, antigen, fibrin, fibrinogen, thrombospondin, fibronectin, laminin, or vimentin. In another aspect, the cells or platelets are obtained from a suspect suspected of having von Willebrand disease, platelet type von Willebrand disease, acquired von Willebrand syndrome, hypertrophic cardiomyopathy, Bernard-Soulier syndrome, Glanzmann's thrombasthenia thrombocytopenia, or various autoimmune coagulation disorders. In another aspect, the cells or platelets are obtained from a suspect suspected of having coagulopathies associated with left ventricular assist device implantation. In another aspect, the surface is in a multifluidic chamber. In another aspect, the surface is coated with a truncated human von Willebrand factor fusion protein that comprises a human von Willebrand factor platelet adhesion domain and a recombinant binding domain. In another aspect, the processor calculates the dynamic range of platelet factor to distinguish between two or more low adhesion cells or platelet binding dysfunctions that is statistically significant. In another aspect, the apparatus distinguishes between different low adhesion cell or platelet binding dysfunctions that is statistically significant. In another aspect, the cells are selected from T cells, B cells, macrophages, neutrophils, basophils, or eosinophils. In another aspect, the agent comprises an ethylene glycol polymer bound to the surface and a bivalent cation to provide a consistent binding surface for a His-tag or equivalent bivalent cation binding peptide or polypeptide that is removably attached with high affinity to the bivalent cation. In another aspect, wherein the apparatus processes between 100 to 1000, 1000 to 10,000, 10,000 to 15,000, 25,000 to 50,000, 50 to 75,000, 75,000 to 100,000, 100 to 100,000, 1000 to 90,000, 20,000 to 80,000, 30,000 to 70,000, 40,000 to 60,000 or more than 100,000 cells or platelets are imaged and processed to determine the median and the mean roll time of cells or platelets is determined to calculate the level of adhesion of the cells or platelets to the agent on the surface. In another aspect, the dynamic range is in the low adhesion level. In another aspect, the dynamic range is in the low adhesion level and provides differentiation between various low adhesion point mutants in von Willebrand disease. In another aspect, the processor calculates the adhesion and determines if the von Willebrand factor is natively structured, native-like 2M, native-like 2B, molten globule 2M or molten globule 2B. In another aspect, the processor distinguishes between a native von Willebrand Factor A1 domain and one or more of the following mutations: G1324S (2M); A1437T (2M); R1308L (2B); R1341Q (2B); R1306Q (2B); I1372S (2B); I1309V (2B); F1369I (2M); E1359K (2M); I1425F (2M); S1285F (2M); R1374H (2M); H1268D (2B); or V1316M.

In another embodiment the present invention includes a method of using a real-time quantitative measurement of a dynamic range of cell or platelet function to select a treatment comprising: obtaining a cell or platelet sample from a subject suspected of having a dysfunction in cell or platelet adhesion; flowing the cell or platelet sample over a rheological shear flow surface, wherein the surface is coated with an agent that provides cell or platelet binding; measuring the transit of the cell or platelet sample on the surface under shear flow; calculating both a pause time and a roll time of the cells or platelets obtained from a subject suspected of having a cell or platelet dysfunction, wherein the processor determines a median and a mean pause time and a median and a mean roll time of cells or platelets and compares them to cells or platelets having at least one of: no cell or platelet adhesion dysfunction, a mild adhesion dysfunction, or a severe adhesion dysfunction in a cell surface adhesion molecule, wherein the processor provides a real-time quantitative measurement of a dynamic range of cell or platelet function; and based on the dynamic range of cell or platelet function from the cell or platelet sample determining the course of treatment for the subject. In one aspect, the cell surface adhesion molecule is von Willebrand factor, an ICAM, a VCAM, a Lectin, an Integrin, a collagen, a fibrinogen, a subendothelial membrane protein, glycoprotein Ib(β), glycoproteinIb(a), glycoprotein Ib-IX-V complex, glycoprotein IIbIIIa, glyprotein VI, major histocompatibility complex, integrin α2 β1, or immunoglobin. In another aspect, the cell surface adhesion molecule is von Willebrand factor and the mutations are point mutations in the A1, A2, A3, B, or C domains. In another aspect, the surface is coated with a mutant human von Willebrand factor. In another aspect, the cells or platelets are obtained from a suspect suspected of having von Willebrand disease, platelet type von Willebrand disease, acquired von Willebrand syndrome, hypertrophic cardiomyopathy, Bernard-Soulier syndrome, Glanzmann's thrombasthenia thrombocytopenia, or various autoimmune coagulation disorders. In another aspect, the cells or platelets are obtained from a suspect suspected of having coagulopathies associated with left ventricular assist device implantation. In another aspect, the surface is in a multifluidic chamber. In another aspect, the surface is coated with a truncated human von Willebrand factor fusion protein that comprises a human von Willebrand factor platelet adhesion domain and a recombinant binding domain. In another aspect, the processor calculates the dynamic range of cell or platelet adhesion molecule to distinguish between two or more low adhesion cell or platelet binding dysfunctions that is statistically significant. In another aspect, the method distinguishes between different low adhesion cell or platelet binding dysfunctions that is statistically significant. In another aspect, the cells are selected from T cells, B cells, macrophages, neutrophils, basophils, or eosinophils. In another aspect, the agent comprises an ethylene glycol polymer bound to the surface and a bivalent cation to provide a consistent binding surface for a His-tag or equivalent bivalent cation binding peptide or polypeptide that is removably attached with high affinity to the bivalent cation. In another aspect, the method processes data from between 100 to 1000, 1000 to 10,000, 10,000 to 15,000, 25,000 to 50,000, 50 to 75,000, 75,000 to 100,000, 100 to 100,000, 1000 to 90,000, 20,000 to 80,000, 30,000 to 70,000, 40,000 to 60,000 or more than 100,000 cells or platelets are imaged and processed to determine the median and the mean roll time of cells or platelets is determined to calculate the level of adhesion of the cells or platelets to the agent on the surface. In another aspect, the dynamic range is in the low adhesion level. In another aspect, the dynamic range is in the low adhesion level and provides differentiation between various low adhesion point mutants in von Willebrand disease. In another aspect, the method further comprises the steps of: obtaining cell or platelet position data at a first time for many translocation events, wherein each event represents the 2-dimensional movement of a single cell or platelet moving over time; obtaining additional, subsequent position data for each of the cells or platelets at a second or subsequent point in time; calculating distance trajectories from coordinate data as a function of time and numerically differentiated using a Savitzky-Golay algorithm to obtain instantaneous velocities and accelerations as a function of time for every moving cell or platelet; reporting the properties are instantaneous velocities and accelerations as a function of time for each X and Y component directions; calculating pause times from the amount of time a cell or platelet is motionless, a distribution of the pause times, velocities and accelerations; and plotting the pause times and shear rates calculated from the pause times, velocities and accelerations to determine the level of adhesion of the cells or platelets to the surface. In another aspect, the processor calculates the adhesion and determines if the von Willebrand Factor is natively structured, nativelike 2M, nativelike 2B, molten globule 2M or molten globule 2B. In another aspect, the processor distinguishes between a native von Willebrand Factor A1 domain and one or more of the following mutations: G1324S (2M); A1437T (2M); R1308L (2B); R1341Q (2B); R1306Q (2B); I1372S (2B); I1309V (2B); F1369I (2M); E1359K (2M); I1425F (2M); S1285F (2M); R1374H (2M); H1268D (2B); or V1316M.

Yet another embodiment of the present invention includes a method of evaluating a candidate drug for changing cell or platelet adhesion comprising: (a) measuring the transit of a cell or platelet sample on a rheological shear flow surface under shear flow from a patient having a platelet adhesion dysfunction; (b) calculating both a pause time and a roll time of cells or platelets of the cell or platelet sample to determine a median and a mean pause time and a median and a mean roll time of cell or platelets; (c) comparing the median and mean pause times and the median and mean roll times of the cell or platelets in the cell or platelet sample to a sample obtained from a subject that does not have a cell or platelet adhesion dysfunction to provide a real-time quantitative measurement of a dynamic range of cell or platelet function; (d) exposing the platelets from the cell or platelet sample to a candidate drug; (e) repeating steps (a) to (c) after the exposing the cells or platelets to the candidate drug; and (f) determining if the candidate drug changes the median and mean pause times and the median and mean roll times of the cells or platelets that is statistically significant as compared to any reduction in the cells or platelets from the subject that does not have a cell or platelet adhesion dysfunction, wherein a statistically significant reduction indicates that the candidate drug is useful in changing cell or platelet adhesion.

Another embodiment of the present invention includes a method of performing a clinical trial to evaluate a candidate drug believed to be useful in treating a bleeding diatheses, the method comprising: (a) measuring the transit of a cell or platelet sample on a rheological shear flow surface under shear flow from a set of patients; (b) calculating both a pause time and a roll time of cells or platelets of the cell or platelet sample to determine a median and a mean pause time and a median and a mean roll time of cells or platelets; (c) comparing the median and mean pause times and the median and mean roll times of the cells or platelets in the platelet sample to provide a real-time quantitative measurement of a dynamic range of platelet function; (d) administering a candidate drug to a first subset of the patients, and a placebo to a second subset of the patients; (e) repeating steps (a) to (c) after the administration of the candidate drug or the placebo; and (f) determining if the candidate drug changes the median and mean pause times and the median and mean roll times of the cells or platelets that is statistically significant as compared to any reduction occurring in the second subset of patients, wherein a statistically significant reduction indicates that the candidate drug is useful in treating the bleeding diatheses.

Yet another embodiment of the present invention includes a method of using a real-time quantitative measurement of a dynamic range of cell or platelet function to select a treatment comprising: obtaining a cell or platelet sample from a subject suspected of having a dysfunction in platelet adhesion; flowing the cell or platelet sample over a rheological shear flow surface, wherein the surface is coated with an agent that provides cell or platelet binding; measuring the transit of the cell or platelet sample on the surface under shear flow; calculating both a pause time and a roll time of the cells or platelets obtained from a subject suspected of having a cell or platelet dysfunction, wherein the processor determines a median and a mean pause time and a median and a mean roll time of cells or platelets and compares them to platelets having at least one of: no cell or platelet adhesion dysfunction, a mild adhesion dysfunction, or a severe adhesion dysfunction in a cell surface adhesion molecule, wherein the processor provides a real-time quantitative measurement of a dynamic range of cell or platelet function; plotting the pause time and roll times for the cells or platelets, wherein the plots distinguish between various low adhesion diseases or conditions; and based on the dynamic range of cell or platelet function from the cell or platelet sample determining the course of treatment for the subject.

### BRIEF DESCRIPTION OF THE DRAWINGS

For a more complete understanding of the features and advantages of the present invention, reference is now made to the detailed description of the invention along with the accompanying figures and in which:

FIG. 1A shows the structural alignment of WT A1 (1auq, black), type 2B I1309V A1 (1ijk, dark grey) and type 2B R1306Q A1 extracted from the co-crystal complex with GP1bα (1m10, light grey). FIG. 1B shows the root mean squared deviation (RMSD) of all backbone atoms. Top: R1306Q—WT. Bottom: I1309V—WT. Diffraction resolutions are 2.3 Å for WT, 3.1 Å for R1306Q and 1.8 Å for I1309V. Horizontal line indicates residues within the disulfide bond. FIG. 1C shows the set-up of the present invention, in which a parallel plate flow chamber was used to examine the physics of shear-induced adhesion, in this example, of platelets.

FIG. 2 is a graph of platelet pause times and translocation velocities (Inset) for A1-1261 (black circles), A1-1238 (open circles) and A1-1261 in presence of 66 μM N-terminal peptide (black squares). The number of platelets analyzed at each shear rate is in the lower panel. Data are representative of three independent studies from different donors. Both proteins were immobilized at a concentration of 5 μM for ˜2 h and 300 μM of N-terminal peptide were added to immobilized A1-1261 followed by 1 h incubation. N-terminal peptide was added to 1 mL whole blood and buffer to a final concentration of 66 μM prior to flow.

FIG. 3A shows a far-UV CD spectra at 20° Celsius of A1-1261 (black circles), A1-1238 (open circles) and Inset: the N-terminal peptide (black squares) and of the scrambled peptide (open squares). FIG. 3B shows the urea denaturation at 25° Celsius of A1-1261 (black circles) and of A1-1238 (open circles) via CD at λ=222 nm. Insets are the ΔG⁰, m-value and cₘ for the native to intermediate transition. A1-1261 (black bars) and A1-1238 (white bars).

FIGS. 4A to 4D show scan rate dependency of normalized thermal unfolding transitions by fluorescence (λλ=359 nm) of A1-1261 (FIG. 4A) and A1-1238 (FIG. 4B) with excitation λ=280 nm. Scan rates were 2.0° Celsius/min (black circles), 1.6° Celsius/min (open circles), 0.9° Celsius/min (black squares) and 0.4° Celsius/min for A1-1261 and 0.6° Celsius/min for A1-1238 (both with open squares). Insets show representative thermal scans and corresponding baselines before data normalization. Scan rate dependency of thermal unfolding transitions by CD at λ=222 nm for A1-1261 (FIG. 4C) and A1-1238 (FIG. 4D). Scan rates were 2.0° Celsius/min (black circles), 1.5° Celsius/min (open circles), 1.0° Celsius/min (black squares) and 0.5° Celsius/min (open squares).

FIGS. 5A to 5D shows the parameters obtained from fitting the thermal transitions in FIG. 4A to 4D are reported for each applied method. FIG. 5A—Scan rate dependence of the T*—values (filled symbols and black average and regression lines) and apparent T_{M}—values (open symbols and gray average and regression lines). FIG. 5B—Scan rate dependence of the enthalpy, Δ_{T*}^{‡}. Lines are solid for A1-1261 and dashed for A1-1238. Symbols are: FL, A1-1261 (circles); FL, A1-1238 (squares—dotted squares for the second transition); CD, A1-1261 (triangles); CD, A1-1238 (diamonds). Reported in FIG. 5C and FIG. 5D are the average T* and Δ_{T*}^{‡} values±standard deviation for each method. A1-1261 (black bars); A1-1238 (white bars).

FIG. 6: Excess molar heat capacity by DSC at 2.0° Celsius/min for A1-1261 (black circles) and A1-1238 (open circles). Insets are the transition temperature, T*, and Δ_{T*}^{‡}, enthalpy, of the native to intermediate transition. A1-1261 (black bars) and A1-1238 (white bars).

FIG. 7A—Normalized thermal transitions of tryptophan fluorescence (λ=359 nm) of 2 μM A1-1261 with excitation λ=295 nm as a function of increasing concentrations of free N-terminal peptide. Concentrations are 0 μM (black circles), 0.02 μM (open circles), 0.2 μM (black squares), 50 μM (open squares), 300 μM (black triangles) of the N-terminal peptide and 300 μM of the scrambled peptide (open triangles). The inset shows transitions in presence of 300 μM N-terminal peptide (closed triangles) and in presence of 300 μM scrambled peptide (open triangles).

FIG. 7B—Determination of the binding affinity of the N-terminal peptide to A1-1261 from Δ_{T*}^{‡}. Symbols represent A1-1261+N-terminal peptide (black circles), A1-1261+scrambled peptide (open circles; average—long dashed line) and A1 1238+N-terminal peptide (black squares; average—short dashed line). The lower panel shows T* values for all analyzed thermal scans. Symbols and lines are homolog to the upper panel.

FIGS. 8A to 8F show: FIG. 8A distance travelled by a single platelet over time. Grey line represents the Savitzky-Golay fit of the data (black circles). FIG. 8B the Savitzky-Golay 1st derivative of the distance as a function of time gives the instantaneous velocity of a single platelet over time. Large spikes in the velocity represent detachment events. Parallel horizontal lines represent a threshold around the scatter about zero to define when a platelet is not moving. The length of time at which the velocities are within this threshold is equivalent to the pause time. Higher velocities are rolling velocities. FIG. 8C is a histogram of the velocities attained by a single platelet. Mean and median are indicated. FIG. 8D shows that pause times are determined by the amount of time a platelet's velocity is statistically zero within a noise threshold of 0±0.25 μm/s and all pause time event are averaged over all platelets analyzed. FIG. 8E Shear rate dependence of platelet mean pause times translocating on normal wild-type A1 domain (black) and representative type 2B (white) and 2M (grey) mutations recombinantly engineered into the A1 domain. FIG. 8F shows a rank order of mean pause times for platelets adhering to type 2B and 2M mutated A1 domain relative to WT A1 domain at 1500 s⁻¹. FIG. 8G shows a graph that distinguishes the mean pause times of wild type platelet binding versus a VWD P1337L mutant versus shear rates. FIG. 8H shows a graph that distinguishes the mean pause times of wild type platelet binding versus a VWD H1268D mutant versus shear rates. FIG. 8I shows a graph that distinguishes the mean pause times of wild type platelet binding versus a VWD I1372S mutant versus shear rates. FIG. 8J shows a graph that distinguishes the mean pause times of wild type platelet binding versus a VWD R1450E mutant versus shear rates. FIG. 8K shows a histogram that distinguishes the mean pause times of wild type platelet binding versus VWD R1450E, I1372S, H1268D, and P1337L mutants versus shear rates. FIG. 8L shows a graph that distinguishes the mean pause times of wild type platelet binding versus VWD R1450E, I1372S, H1268D, and P1337L mutants versus shear rates.

FIGS. 9A and 9B show histograms of the median pause times attained for multiple platelets in 1 min movies at two shear rates for A1-1261 (left), A1-1238 (center) and A1-1261+N-terminal peptide (right). FIG. 9A—Shear rate=1025 s⁻¹. FIG. 9B—Shear rate=3200 s⁻¹. n=the total number of platelets.

FIGS. 10A and 10B show histograms of the median velocities attained for multiple platelets in 1 min movies at two shear rates for A1-1261 (left), A1-1238 (center) and A1-1261+N-terminal peptide (right). FIG. 10A—Shear rate=1025 s⁻¹. FIG. 10B—Shear rate=3200 s⁻¹. n=the total number of platelets.

FIGS. 11A to 11C show platelet translocation on disulfide-intact A1 domain as a function of shear rate. FIG. 11A, upper panel shows the mean pause times derived from the statistical average of platelet distributions (filled circles) and from biexponential fitting of the pause time survival fraction using equation (23) (open circles), mean platelet velocities (center panel) and the amount of platelets analyzed (lower panel). FIG. 11B shows the survival fraction decay functions of the platelet pause times with increasing shear rates. Rates are: 800, 1320, 1500, 1950, 2500, 4250 and 9000 s−1 from top to bottom. Curves were fit to a biexponential functions. FIG. 11C shows the apparent rate constants k₁ (black circles) and k₂ (open circles) and the fractional amplitudes associated with k₁ (black circles) and k₂ (open circles). Error bars represent the mean standard deviation from four independent studies.

FIGS. 12A to 12C show a comparison of platelet translocations on disulfide-intact A1 with RCAM A1. FIG. 12A shows the traveled distance of a single platelet as a function of time at 1025 s−1; disulfide-intact A1 (black line), RCAM A1 (grey line). FIG. 12B shows a comparison of the instantaneous velocities obtained for a single platelet interacting with disulfide intact A1 (black line) and RCAM A1 (grey line) at 1025 s−1. FIG. 12C are histograms of the mean velocity of platelets translocating on disulfide-intact A1 (black bars) and RCAM A1 (gray bars) at shear rates of 1025 and 5500 s−1. Total number of platelets analyzed were 526 and 422 for RCAM A1 at 1025 and 5500 s−1 respectively; and 1132 and 1687 for disulfide-intact A1 at 1025 and 5500 s−1 respectively. Data are representative of four independent studies.

FIG. 13A shows fluorescence emission spectra of disulfide-intact A1 (closed symbols) and RCAM A1 (open symbols) in the presence of 0M (circles), 2M (squares) and 8M GdnHCl (triangles) with excitation λ=280 nm. (Inset). Wavelength of maximum fluorescence intensity (λₘₐₓ for disulfide-intact A1 (black bars) and RCAM A1 (white bars). FIG. 13B shows the far UV CD spectra of disulfide-intact A1 (closed circles) in buffer and RCAM A1 (open circles) in 0.5 M GdnHCl. FIG. 13C Near UV CD spectra for disulfide-intact A1 (closed circles) and RCAM A1 in buffer (open circles).

FIGS. 14A and 14B show GdnHCl denaturation (FIG. 14A) and urea denaturation (FIG. 14B) of disulfide-intact A1 (closed circles) and RCAM A1 (open circles) monitored by circular dichroism at λ=222 nm.

FIGS. 15A to 15C show thermal denaturation. FIG. 15A disulfide-intact A1 in 1M urea (closed circles) and RCAM A1 in 1 M urea (closed squares) monitored by circular dichroism at λ=222 nm. FIG. 15B disulfide-intact A1 in buffer (closed circles), 9M urea (open circles) and RCAM A1 in buffer (closed squares) monitored by intrinsic protein fluorescence emission at λₑₓ=359 nm with excitation at λₑₓ=280 nm. FIG. 15C disulfide-intact A1 in buffer (closed circles) and RCAM A1 in buffer (closed squares) monitored by ANS fluorescence emission at λ=495 nm with excitation at λₑₓ=350 nm. (Inset). Spectra of A1 and RCAM A1 in the presence of ANS at 20° Celsius before (closed circles and squares) and after the thermal scan (open circles and squares.

FIG. 16A shows the acrylamide quenching of tryptophan fluorescence in disulfide-intact A1 at 20° Celsius (closed circles) and 1° Celsius (closed squares), RCAM A1 at 20° Celsius (open symbols) and 1 μM NATA at 20° Celsius (grey circles) in buffer. FIG. 16B shows the resulting Stern-Volmer quenching constants derived from the slope of F₀/F as a function of acrylamide molarity at the indicated concentrations of GdnHCl. Disulfide-intact A1 (black bars), RCAM A1 (white bars) and NATA in buffer (grey bar).

FIG. 17 shows the size exclusion chromatograms for native disulfide-intact A1 (closed symbols) and RCAM A1 (open symbols) in buffer (circles) and 0.25M GdnHCl (squares). The column was equilibrated with PGA buffer and calibrated with several proteins as indicated by their peak maxima (a—Ovoalbumin from chicken (44.0 kDa), b—bovine Carboanhydrase (29 kDa), c—Myoglobin from horse (17.0 kDa), d—cytochrome c from horse (12.4 kDa). Void volume=6.85 mL with blue dextran (2000 kDa). Included volume 17.54 mL with vitamin B{12} (1.35 kDa).

FIGS. 18A to 18E summarize the clinical correlation of pause times and varying low level binding interactions with specific VWD mutations. FIG. 18A: the Shear rate dependency of platelet pause times on representative normal (WT A1), type 2B (P1337L) and type 2M (A1437T). FIG. 18B shows the rank order of pause times at 1500 s⁻¹ shear rate from least to greatest. The type 2M variants F1369I, E1359K, I1425F, 51285F and R1374H did not bind platelets at any shear rate. Grey bars indicate “molten globule” type 2B variants of A1 domain. Shear rate dependency of platelet pause times on FIG. 18C “natively structured” type 2B A1 domain variants, FIG. 18D “natively structured” type 2M A1 domain variants, and FIG. 18E “molten globule” A1 domain variants.

FIGS. 19A and 19B show, FIG. 19A are the platelet counts reported for vWD patients with known mutations as a function of the platelet pause times obtained at 1500 s⁻¹ shear rate shown in FIG. 2. The linearity has a correlation coefficient of determination (R2=0:81). FIG. 19B shows the ratio of vWF ristocetin cofactor activity relative to vWF antigen levels reported for vWD patients. Grey areas indicate the normal range of these clinical metrics. 2B=red, 2M=blue. Data points at zero pause time represent the type 2M mutations for which there was no shear dependent binding. These data are also tabulated in Table 4.

FIGS. 20A and 20B show the structure of the A1 domain in complex with GPIba with locations of type 2B (red) and 2M (blue) vWD mutations (A) and colored by conformational class (B); native (same color as A1 structure), nativelike (hatch right), and molten globule (hatch left). (Helices 2 and 3 are also indicated in hatch left.) The tryptophan residue is indicated for reference to FL studies. PDB:1SQ0. CHIMERA, Ver. 8.6.1 (www.cgl.ucsf.edu/chimera/) was used.

FIG. 21 Urea denaturation (FIG. 21A) and thermal denaturation (FIG. 21B) of the A1 domain monitored by CD at 222 nm for the representative variants indicated. (red) Gain-of-function, type-2B. (Blue) Loss-of-function, type-2M.

FIGS. 22A to 22D show urea denaturation of type 2B (22A & 22C) and type 2M (22B & 22D) variants of the A1 domain monitored by far-UV CD and categorized by “Native and Native-Like” (22A & 22B) and “Molten Globule” (22C & 22D).

FIGS. 23A to 23D shows thermal denaturation of type 2B (23A & 23C) and type 2M (23B & 23D) variants of the A1 domain monitored by far-UV CD and categorized by “Native and Native-Like” (23A & 23B) and “Molten Globule” (23C & 23D). Thermal scan rate was 2 C/min.

FIGS. 24A to 24D show thermal denaturation of type 2B (24A & 24C) and type 2M (24B & 24D) variants of the A1 domain monitored by intrinsic protein FL and categorized by “Native and Native-Like” (24A & 24B) and “Molten Globule” (24C & 24D). Thermal scan rate was 2 C/min.

FIGS. 25A to 25D show thermal denaturation of type 2B (25A & 25C) and type 2M (25B & 25D) variants of the A1 domain monitored by ANS FL and categorized by “Native and Native-Like” (25A & 25B) and “Molten Globule” (25C & 25D). Thermal scan rate was 2 C/min. The maximum ANS FL intensity of spectra taken prior to and after thermal denaturation was used to calculate the intensity ratio reported in FIG. 5F of the manuscript, Iₘₐₓ(N)/Iₘₐₓ(D).

FIG. 26 shows the thermal unfolding midpoints correlate with urea denaturation midpoints (R²=0.93).

FIGS. 27A to 27F show the Far-UV CD spectra of type 2B (FIGS. 27A & 27C) and type 2M (FIGS. 27B & 27D) variants of the A1 domain categorized by “Native and Native-Like” (FIGS. 27A & 27B) and “Molten Globule” (FIGS. 27C & 27D). FIG. 27E near-UV CD spectra of A1 domain variants from each of the four structure function classes represented in panels 27A-D.

FIGS. 28A to 28F show the biophysical metrics for secondary and tertiary structure. (A) Far-UV CD spectra. (B) CD ellipticity at 222 nm. (C) Stern-Volmer plots. (D) Stern-Volmer collisional quenching constants. (E) Normalized ANS FL spectra of native and thermally denatured A1 domain variants. (F) ANS intensity ratios. (Red) Type 2B and (blue) type 2M in panels A, C, and E (B, D, and F). All variants are ranked according to increasing pause time as in FIG. 31. (Black) Natively structured variants. (Dark gray) Native-Like variants. (Light gray) Molten globule variants. (White) RCAM A1.

FIGS. 29A to 29D shows Stern-Volmer plots of type 2B (29A & 29C) and type 2M (29B & 29D) variants of the A1 domain monitored by intrinsic protein FL and categorized by “Native and Native-Like” (29A & 29B) and “Molten Globule” (29C & 29D). Data were fit with a second order polynomial and the Stern-Volmer quenching constants reported in FIG. 31D of the manuscript were obtained by initial slope (i.e., the first derivative evaluated at 0M acrylamide.)

FIGS. 30A to 30C show a correlation between biophysical metrics for secondary and tertiary structure. (FIG. 30A) Stern-Volmer constants versus ANS intensity ratios (R²±0.82). (FIGS. 30B and 30C) Ellipticity versus ANS intensity ratios (R²±0.77) and Stern-Volmer constants (R²±0.82). (Red) 2B; (blue) 2M; (white) RCAM A1. To see this figure in color, go online.

FIG. 31A Shear-rate dependency of platelet pause times on normal (WT A1), type 2B (P1337L), and type 2M (A1437T) variants. FIG. 31B Rank order of pause times at 1500 s⁻¹ shear rate from least to greatest. The type 2M variants F1369I, E1359K, I1425F, S1285F, and R1374H did not bind platelets at any shear rate. (Solid) Natively structured variants. (Dark shaded) Native-Like variants. (Light shaded) Molten globule variants. (Open) RCAM A1 represents a case of firm adhesion with no platelet translocation due to loss of the disulfide bond (19). Shear-rate dependency of platelet pause times on FIG. 31C natively structured type 2B A1 domain variants, FIG. 31D natively structured type 2M A1 domain variants, and FIG. 31E molten globule A1 domain variants.

FIG. 32A shows platelet counts reported for vWD patients with known mutations as a function of the platelet pause times obtained at 1500 s⁻¹ shear rate shown in FIG. 31. The linearity has a correlation coefficient of determination (R²±0.88). FIG. 32B shows the ratio of vWF ristocetin cofactor activity relative to vWF antigen levels reported for vWD patients. (Gray areas) Normal range of these clinical metrics. (Red) Type 2B; (blue) type 2M. Data points at zero pause time represent the type 2M mutations for which there was no shear-dependent binding. These data are also tabulated in Table 4 with the primary clinical data given in Table 4.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “cell adhesion” or “cell attachment” are used interchangeably to refer to cell or platelet binding to a substrate. Non-limiting examples of cell adhesion can occur between cognate binding pairs or between cell or platelet surface proteins and the substrate or agents attached to the substrate (for example protein-protein binding, bindings between lectins and carbohydrates, and the like), including but not limited to cell adhesion proteins, antibodies, antigens, surface modified plastic and other like substrates.

As used herein, the term “agent,” which may be used interchangeably with the terms “chemical”, “inhibitor” or “drug,” refers to any compound that can be applied to cells or platelets to change their adhesion characteristics, e.g., adhesion strength, pause time, rolling time, rolling distance, etc., as measured and/or described herein.

As used herein, the term “inhibitor” refers to a molecule that blocks or decreases the adherence of cells or platelets to the cognate or other binding pairs or agents described herein, e.g., the binding of a protein to another protein, to itself or with other molecules such as carbohydrates.

As used herein, the term “mild adhesion dysfunction” refers to the extent to which cells or platelets bind to an endothelial or subendothelial vessel (or a surface modified to mimic an endothelial or subendothelial vessel) or other surface to which the cells or platelets would adhere to in vivo or in vitro that is less than the normal or wild-type binding of the native protein on cells or platelets with the equivalent surface. One non-limiting example of a mild adhesion dysfunction includes VWD mutants R1450E, I1372S, H1268D, and P1337L, which demonstrate varying degrees of mild adhesion dysfunction versus wild-type or normal binding by vWF, as known in the artor as described hereinbelow with regard to vWD.

### Example 1

### The Linker Between the D3 and A1 Domains of vWF Suppresses A1-GPIbα Catch Bonds by Site-Specific Binding to the A1 Domain

Platelet attachment to von Willebrand factor (vWF) requires the interaction between the platelet GP1bα and exposed vWF-A1 domains. Structural insights into the mechanism of the A1-GP1bα interaction have been limited to an N-terminally truncated A1 domain that lacks residues Q₁₂₃₈-E₁₂₆₀ that make up the linker between the D3 and A1 domains of vWF. The inventor has demonstrated that removal of these residues destabilizes quaternary interactions in the A1A2A3 tri-domain and contributes to platelet activation under high shear (Auton et al., J Biol Chem 2012; 287:14579-14585). This example demonstrates that removal of these residues from the single A1 domain enhances platelet pause times on immobilized A1 under rheological shear. A rigorous comparison between the truncated A1-1261 and full-length A1-1238 domains demonstrates a kinetic stabilization of the A1 domain induced by these N-terminal residues as evident in the enthalpy of the unfolding transition. This stabilization occurs through site and sequence specific binding of the N-terminal peptide to A1. Binding of free N-terminal peptide to A1-1261 has an affinity K_{D}=46 μM and this binding although free to dissociate is sufficient to suppress the platelet pause times to levels comparable to A1-1238 under shear stress. Our results support a dual structure/function role for this linker region involving a conformational equilibria that maintains quaternary A domain associations in the inactive state of vWF at low shear and an intra-A1-domain conformation that regulates the strength of platelet GP lba-vWF A1 domain associations in the active state of vWF at high shear.

The multimeric plasma glycoprotein, von Willebrand factor (vWF), is secreted from vascular endothelial cells into the blood and subendothelium as long filaments of covalently coupled monomeric units, each containing a conformationally regulated hook for platelet attachment. These monomeric units each contain a series of domains arranged in the order, D′-D3-A1-A2-A3-D4-B1-B2-B3-C1-C2-CK (Bonthron:1986, Sadler:1998). Within this sequence, the A1 domain functions as the hook for capturing platelets through interaction with platelet glycoprotein 1b and contributes to the arrest of bleeding under the vascular shear stress of blood flow (Savage:1996).

Early studies of the domain structure of vWF isolated a tryptic fragment between residues V₁₂₁₂-K₁₄₉₁ that retained functional binding to platelet GPlb (Fujimura:1986, Fujimura:198). In a variety of studies designed to assess platelet adhesion, heparin and collagen binding, this sequence was engineered recombinantly by several investigators with various lengths of the N and C termini outside of the major disulfide loop that comprises the A1 domain (Pietu:1989, Sugimoto:1991, Gralnick:1992, Azuma:1991). Eventually, optimal lengths of these N and C termini were obtained in a construct spanning residues Q₁₂₃₈-P₁₄₇₁ that eliminated disulfide linked aggregates and improved expression, purification and solubility of the recombinant A1 domain as a monomeric species in solution (Cruz:1993). For the better part of the last two decades, this sequence has been demonstrated to retain the regulatory mechanism for platelet adhesion in response to the shear stress of blood flow. It has been demonstrated to form high strength bonds with platelet GP1b (Arya:2002, Arya:2005), shear dependent platelet rolling on A1 domain coated surfaces (Coburn:2011, Yago:2008) and force dependent single molecule catch bonds with platelet GP1bα (Yago:2008). Furthermore, it has provided internally consistent relationships between the interdependence of conformational thermodynamics with shear and force dependent binding of platelet GP1bα based on clinically identified mutations that cause opposite functional phenotypes in von Willebrand disease (vWD) (Auton:2010, Auton:2009).

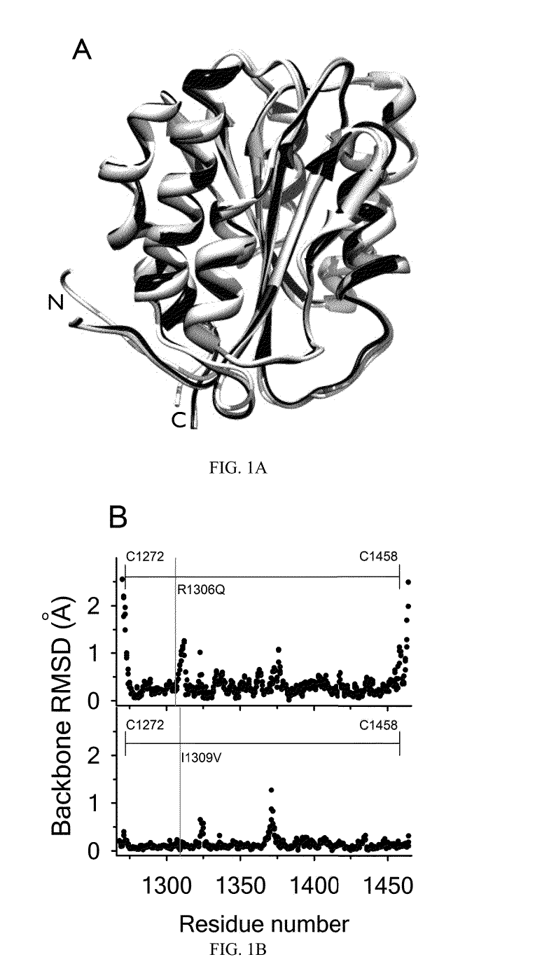

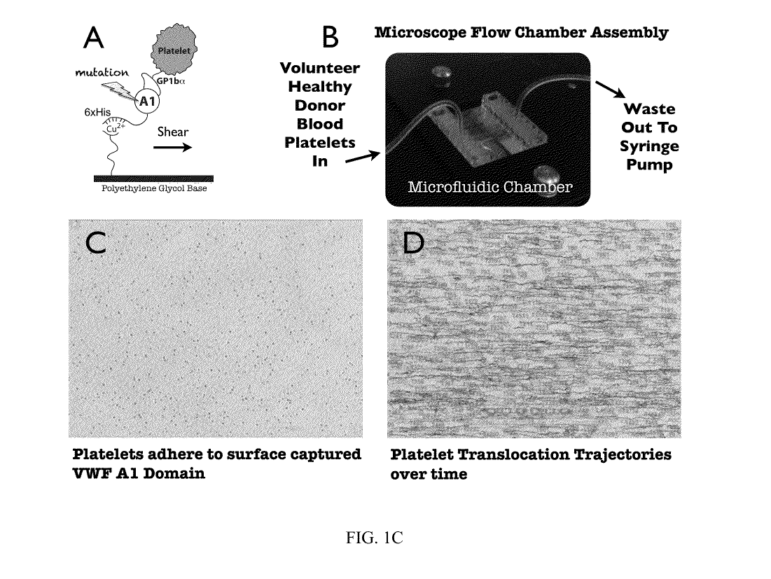

Early efforts to obtain a crystal structure of this domain were unsuccessful until chymotrypsin was used to cleave off residues Q₁₂₃₈-Y₁₂₅₈ and diffractable crystals were obtained (Emsley:1998, Fukuda:2002). While this truncated domain spanning the structurally resolved residues D₁₂₆₁-P₁₄₇₁ can bind to GP1bα, platelet adhesion to it at high shear rates was not as efficient as the domain containing the N-terminal flanking region which showed similar adhesive activity as native vWF (Kim:2010, Miyata:1999). Subsequent crystal structures of the truncated A1 domain and its co-crystal complex with GP1bα with and without type 2 vWD mutations show that the structures are comparatively similar with the backbone RSMD less than 1 \AA over much of the sequence within the disulphide loop (FIG. 1) (Celikel:2000, Fukuda:2002, Huizinga:2002, Dumas:2004). Consequently, structural insights into the mechanisms of type 2 von Willebrand Disease have been inadequate.

It has been known since the late 1980's that 15 residue peptides spanning the region between L₁₂₃₂-D₁₂₆₁ inhibit greater than 50% of the binding of vWF to platelet GP1bα. Furthermore, these peptides can completely inhibit the interaction between vWF and two monoclonal antibodies (NMC-4 and RG-46) known to block ristocetin and botrocetin-induced binding of vWF to GP1bα (Mohri:1988, Fujimura:1991). Co-crystal structures of A1 in complex with NMC-4 moAb show that NMC-4 binds A1 at the α4-α5 helix-loop-helix (Celikel:1998). Given the location of the structurally resolved N-terminal residues beginning at D₁₂₆₁, these data suggest that inhibition of the A1:NMC-4 interaction by the sequence Q₁₂₃₈-E₁₂₆₀ would involve a significant wrapping of the N-terminal sequence around the A1 domain. This type of interaction could result in a shielding effect that inhibits binding between A1 and GP1bα as has been demonstrated using vWF A domain fragments containing the N-terminal D′D3 domains (Ulrichts:2006). What is clear is that this N-terminal sequence, Q₁₂₃₈-E₁₂₆₀, is highly dynamic and its inhibition of crystal growth has precluded an accurate structure-based mechanism of GP1bα binding because of the inability to resolve its conformation in complex with the A1 domain.

The present inventor has shown that truncation of this N-terminal sequence destabilizes domain associations within the A1A2A3 tridomain resulting in a moderate increase in avidity for platelet GP1bα and fibrinogen-dependent platelet activation at high shear (Auton:2012). Similarly, moAb 1C1E7 directed at the N-terminal sequence induces a vWF functional phenotype similar to type 2B vWD with enhanced RIPA, SIPA, and evidence of platelet activation via increased intracellular Ca²⁺ (Tornai:1993, Ulrichts:2004). Here the inventor shows using flow chamber studies that this N-terminal flanking sequence suppresses the catch bond by decreasing the pause time of platelet interactions with surface immobilized A1 domain. Furthermore, the inventor developed a thermodynamic comparison of the stability of the A1 domain with and without its N-terminal flanking region and demonstrate that A1 binds specifically to the N-terminal peptide in solution. The binding of the free N-terminal peptide by A1 lacking this sequence also suppresses the catch bond.

Platelet Adhesion to Immobilized A1 Under Shear Flow.

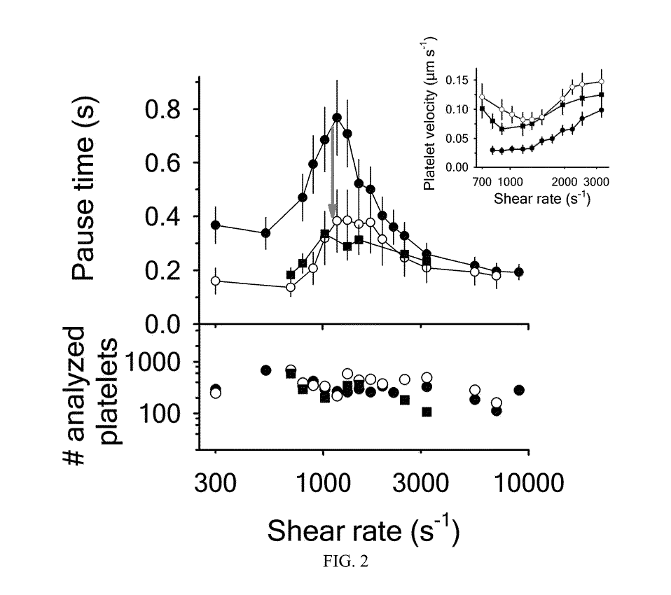

To understand the effect of the N-terminal peptide on the function of the A1-domain of vWF, the protein was expressed with (A1-1238) and without the N-terminal peptide (A1-1261). The inventor used a flow chamber to measure the dynamics of platelet interactions with A1-1238 and A1-1261. Each domain variant was immobilized on glass slides with a modified Cu²⁺ chelating chemistry that enabled surface capture via the N-terminally fused histidine tag. This method ensured reproducible capture of the proteins eliminating possible structural issues associated with nonspecific immobilization on plastic or glass. The slides were completely inert to platelets. Next, 1 mL of citrated whole blood followed by Tris buffered saline was perfused at low shear to enable platelets to attach to the surface immobilized domains. After red cells were perfused away with buffer, the translocation of the remaining attached platelets over the surfaces was recorded at each shear rate and the velocities and pause times calculated from the coordinate data of each platelet on the surface as described in the methods. FIG. 2 shows the pause time data as a function of shear rate along with the number of platelet tracks analyzed from each movie. A1-1261 has longer pause times than A1-1238 indicating that the presence of the covalently linked N-terminal sequence decreases the time for which platelets remain attached. In addition, the translocation velocities were increased for A1-1238 relative to A1-1261. To determine if this N-terminal sequence could inhibit the catch bond when free in solution, the inventor incubated surface immobilized A1-1261 with excess free peptide and added the free peptide to 1 mL of whole blood and to our perfusion buffer and performed the assay. The result was a significant decrease in pause times to the levels of that obtained for A1-1238 and an increase in translocation velocities. Addition of free N-terminal peptide to A1-1238 or a peptide with the same amino acid composition, but a scrambled sequence, to either A1-1238 or A1-1261 did not significantly change the pause times or translocation velocities observed in the absence of peptide.

Structural Characterization

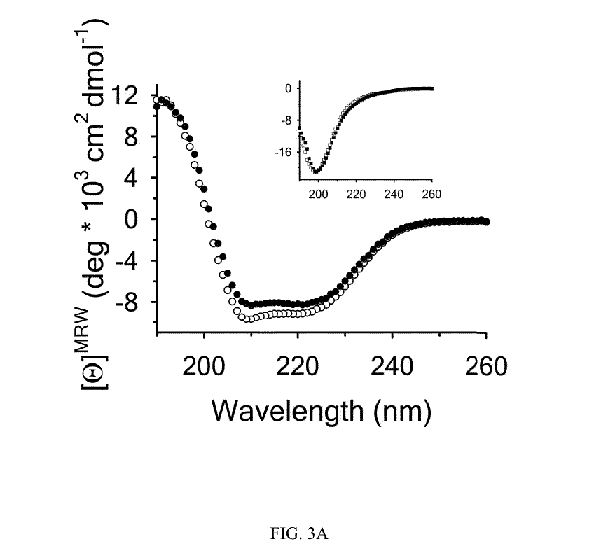

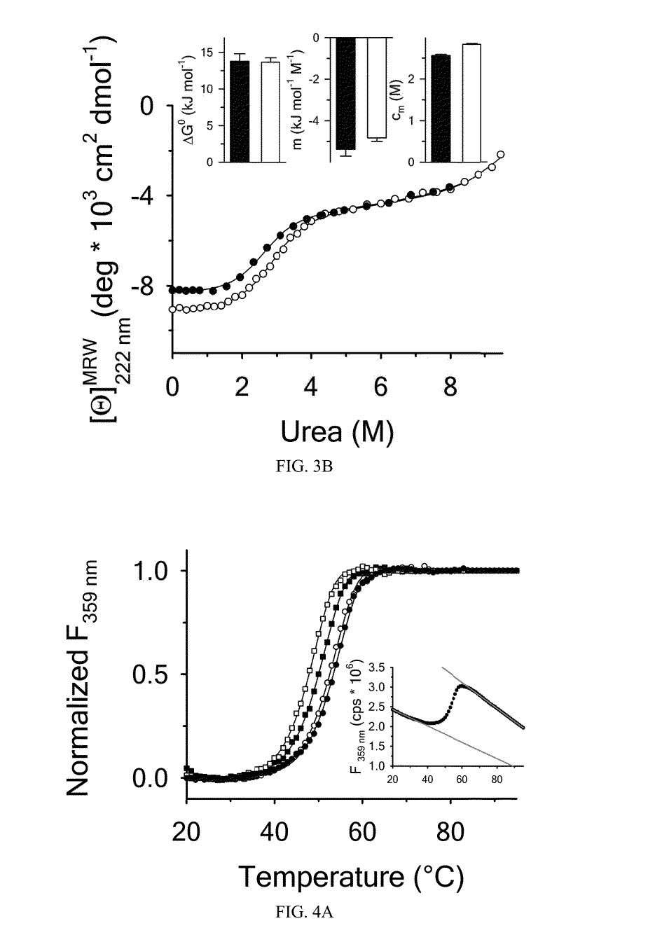

CD spectra obtained for A1-1261 and for A1-1238 are shown in FIG. 3A. Both proteins show a spectrum dominated by relatively high α-helical content as indicated by the two minima at λ=222 nm and 208 nm. Comparatively, the ellipticity of A1-1261 is slightly reduced relative to A1-1238, but a calculation of the secondary structure contributions for the two proteins (Supplement Table 1) shows similar α-helical and parallel and anti-parallel β-sheet structure (Bohm:1992). By contrast, the spectrum of the N-terminal peptide is dominated by the contribution of random coil-structure and spectrally equivalent to a peptide containing a scrambled sequence but identical amino acid composition.

Urea-Induced Unfolding

A1-1261 and A1-1238 were chemically unfolded with urea in order to compare the obtained thermodynamic parameters. The unfolding was monitored at 25° Celsius by CD at λ=222 nm, FIG. 3B. For both proteins, unfolding through an intermediate state was observed, as has been previously reported (Auton:2009). The thermodynamic parameters are illustrated in the insets of FIG. 3B and listed in the Supplement Table 2. Relative to A1-1261, A1-1238 has a marginal loss in unfolding cooperativity (+0.55±0.38 kJ/mol/M) of the transition and the midpoint, cₘ, of A1-1238 is increased by +0.27±0.04 M. These two contributions to the ΔG⁰ cancel each other so that the N-terminal peptide is neither stabilizing nor destabilizing the conformational transition.

Temperature-Induced Unfolding

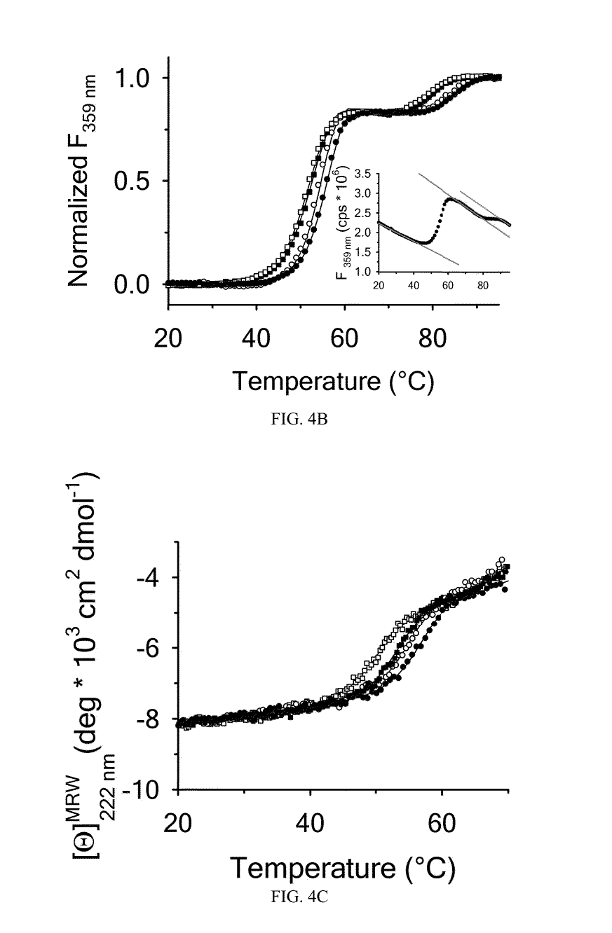

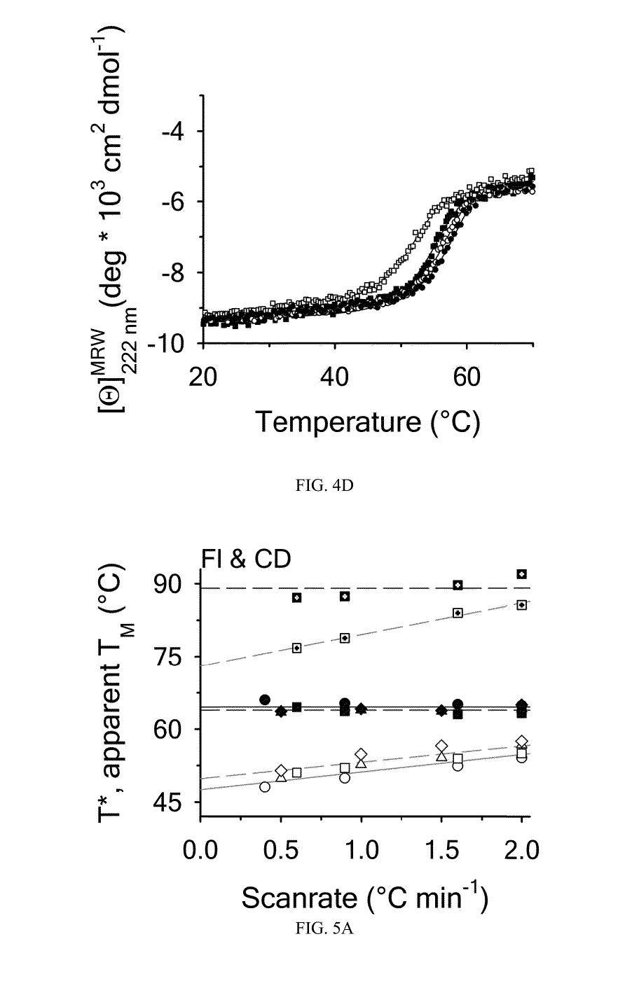

A1-1261 and A1-1238 were thermally unfolded using intrinsic protein tyrosine and tryptophan fluorescence with an excitation λ=280 nm and emission λ=359 nm and CD monitored at λ=222 nm. The observed thermal scans shown in FIGS. 4A and 4D demonstrate a strong dependency of the primary unfolding transition on the rate at which the temperature is changed indicating that the unfolding is kinetically controlled. As the scan rate increases, the midpoint of the transition (apparent T_{M}) also increases. Closer inspection of the thermal transition in FIG. 4B shows that while both spectroscopic methods capture the primary unfolding event between 40° Celsius and 60° Celsius, fluorescence captures a second transition at high temperature in A1-1238 (FIG. 4B) which is also scan rate dependent. This high temperature transition is absent in A1-1261 suggesting that the removal of the N-terminal sequence further exposes fluorophors in the protein (FIG. 4A). Thermal scans of the peptide alone did not reveal any evidence of a conformational transition by either CD or fluorescence indicating that this high temperature transition results from the exposure of the tyrosine in the N-terminal peptide when covalently linked to the A1 domain.

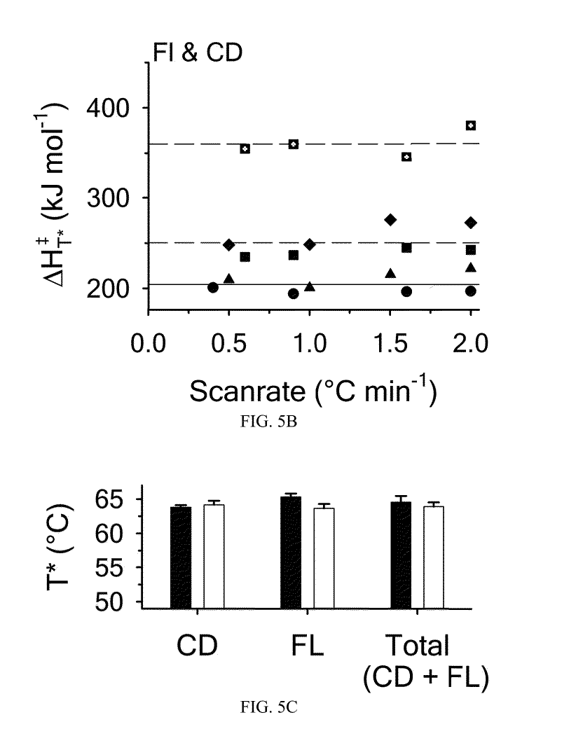

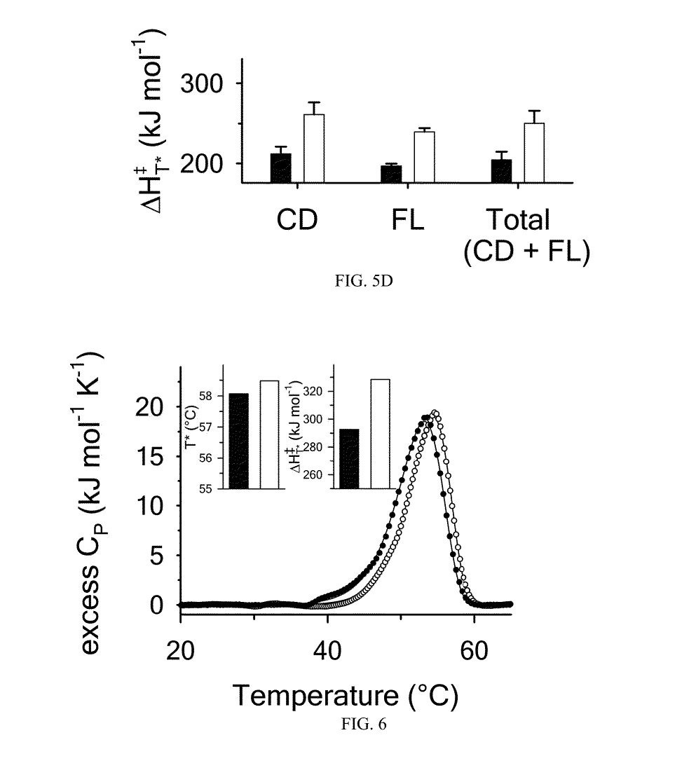

Thermal transitions were analyzed in terms of the observed midpoint of the transition, apparent T_{M}, and also by a two-state irreversible model or a three-state irreversible model as described in the Supplement and the calculated parameters are summarized in FIGS. 5A to 5D. FIG. 5 demonstrates the increasing apparent T_{M} as a function of scan rate for all methods employed. If the protein unfolding was at equilibrium and reversible, this apparent T_{M} would remain constant at all scan rates. Extrapolation of the T_{M} to a scan rate of 0° Celsius/min provides an indication of where reversible unfolding would occur under equilibrium conditions; 50° Celsius for A1-1238 and 48° Celsius for A1-1261 from both CD and fluorescence data. For the second transition observed by fluorescence that occurs with A1-1238, the T_{M} extrapolates to about 73° Celsius in the limit of zero scan rate. Analysis by the two-state and three-state irreversible models yields an R²>0.99 for both fluorescence and CD and a transition temperature, T*, that is independent of the temperature scan rate. In addition, the enthalpies of the transitions are independent of scan rate within experimental error FIG. 5B. FIGS. 5C and 5D show the resulting average T* and ΔH_{T*}^{‡}±the standard deviation obtained from the fitting of the primary thermal transitions in FIG. 4A to 4D for CD and fluorescence and a total average for both methods. Comparing the transition temperatures for the primary unfolding transition shows that both A1-1261 and A1-1238 have an identical T* within experimental error. A1-1261 T*=64.5±0.9° Celsius is not significantly different than A1-1238 T*=63.9±0.6° Celsius. However, the unfolding enthalpy was statistically larger for A1-1238 (250±15 kJ/mol) than for A1-1261 (204±10 kJ/mol). The average T* and ΔH_{T*}^{‡} observed in the second fluorescence transition of A1-1238 is 89.1±2.2° Celsius and 360±14 kJ/mol.

Thermal unfolding at 2° Celsius/min by DSC also shows that the T* is not significantly different between A1-1261 and A1-1231, but the enthalpy of unfolding is increased for A1-1238 (FIG. 6). The values obtained via DSC were not quantitatively comparable to those obtained from CD and fluorescence as a result of the aggregation of both proteins at the high temperatures and concentrations about 50 μM required to obtain reliable signal/noise in the calorimetric power compensation. It should also be noted that all thermal transitions remained irreversible in the presence of 1M arginine, an excipient known to solubilize proteins, and a scan rate dependency was also observed in the presence of 1M urea confirming that thermal unfolding is intrinsically irreversible and not an artifact of any nonspecific aggregation.

Interaction of A1-1261 with the N-Terminal Peptide

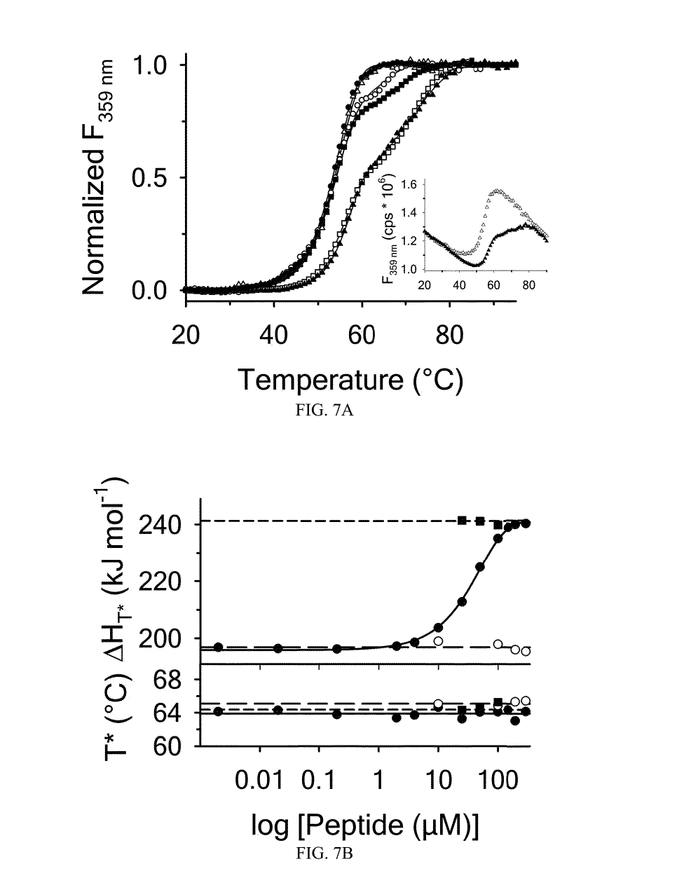

Since the thermal denaturation of A1 revealed that the covalently linked N-terminal peptide increases the enthalpy of the unfolding transition of the A1 domain, it was of interest whether this property could be used to detect binding of the peptide in solution to A1-1261. This would imply a specific interaction between this N-terminal sequence and the A1 domain when covalently linked in sequence. The only way to examine such a stabilization using spectroscopy was the measurement of thermal unfolding via fluorescence. Since the N-terminal peptide also contains a single tyrosine residue, the excitation wavelength was changed to λ=295 nm in order to measure tryptophan fluorescence and the effect of the peptide on the unfolding of the A1-domain was observed indirectly. Thermal scans of 2 μM A1-1261 were performed as a function of increasing amounts of the N-terminal peptide at 2° Celsius/min. FIG. 7A shows that the primary transition shifts from an apparent T_{M} 53° Celsius in the absence of peptide to 55.5° Celsius in the presence of saturating concentrations of peptide. The second transition becomes more prominent as the peptide concentration increases indicating that the peptide is binding to A1-1261. Thermal scans were also done in the presence of a peptide with the same amino acid composition, but a scrambled sequence. The inset of FIG. 7A compares the raw fluorescence thermal scans in the presence of 300 μM scrambled and N-terminal peptides. Unfolding in the presence of excess scrambled peptide was equivalent to unfolding in the absence of either peptide. FIG. 7B shows the resulting ΔH_{T*}^{‡} and T* obtained from these transitions as a function of the total concentration of N-terminal peptide. Whereas the T* was constant within ±1.0° Celsius, the enthalpy of the transition changed sigmoidally on a log scale from ≈197 kJ/mol to ≈242 kJ/mol indicating that the unfolding enthalpy is sensitive to the binding of the N-terminal peptide. Fitting the enthalpy data to a simple single site binding model yields a dissociation constant of ≈46±6 μM. Also the binding of the N-terminal peptide to A1-1261 was sequence specific since the scrambled peptide did not change the unfolding enthalpy at saturating concentrations. The N-terminal peptide did not change the unfolding enthalpy of A1-1238 suggesting that the binding is site specific and the covalently linked peptide occupies the binding site so that free N-terminal peptide cannot bind. In addition, the binding of the N-terminal peptide is fully reversible as excessive equilibrium dialysis of A1-1261 following incubation with 200 \mu M peptide for 1 hour resulted in a thermal scan with a equivalent A1-1261 in the absence of peptide.

The average T* and ΔH_{T*}^{‡} observed for the peptide-induced second fluorescence transition of A1-1261 are 90.1±8.5° Celsius and 142±37.1 kJ/mol. This second transition yields a comparable T* value to A1-1238 (89.1±2.2° Celsius), although is less well defined in cooperativity with a significant drop in ΔH_{T*}^{‡} from 360±14 kJ/mol when covalently linked to 142±37.1 kJ/mol when free to dissociate from the A1 domain over the full range of peptide concentrations studied.

The results of Example 1 demonstrate the following important properties of the A1 domain.

(1) The covalently linked N-terminal peptide suppresses the A1-GPIbα catch bond resulting in decreased pause times and faster translocation velocities. Specific binding of the N-terminal peptide free in solution to A1-1261 results in diminished pause times and enhanced velocities that are comparable to those obtained with A1-1238.

(2) The N-terminal peptide, free in solution, is unstructured with >80% of the sequence having β and random coil content. In its native sequential context, this 23 residue peptide does not significantly alter the overall secondary structure content of the A1 domain. Therefore, this peptide is likely unstructured in the natural sequential context relative to the A1 domain.

(3) Reversible urea denaturation illustrates that the stability of both A1-1261 and A1-1238 have an equivalent thermodynamic stability at 25° Celsius despite small changes in the cₘ and the m-value. Irreversible thermal denaturation illustrates that both A1-1261 and A1-1238 have equivalent transition temperatures T* that define where the rate of unfolding is unity. Interestingly, the enthalpy of unfolding is larger for A1-1238 than A1-1261 which indicates that the rate of unfolding of 1261 to the intermediate state at temperatures lower than the T* will be faster than A1-1238.

(4) Although urea denaturation of A1 is reversible, thermal denaturation is irreversible and kinetically controlled. Kinetic irreversibility is evident by the significant scan rate dependence of the apparent melting temperature. Analysis of CD and fluorescence transitions using an irreversible two-state model defined by a first order rate constant yield transition temperatures that are scan rate independent. Furthermore, the observed thermal transitions represent the native to intermediate conformational change observed in urea denaturation. This is evident by the significant amount of CD ellipticity remaining in the thermally denatured state and the presence of a second high temperature transition from the intermediate state to denatured observed by fluorescence when excited at 280 nm. The fact that this high temperature transition is not observed with A1-1261 indicates that the N-terminal peptide is involved in this I->D transition. In addition, extrapolation of the first apparent Tₘ of A1-1238 to zero scan rate (equilibrium) gives 49.8±1.2° Celsius. This is in agreement with the Tₘ=51.1±0.5° Celsius obtained from the reversible<->I urea-temperature phase diagram determined previously (Auton:2009th. The apparent Tₘ of the second transition of A1-1238 is also comparable to that obtained from the I<->D urea-temperature phase diagram although the error associated with that transition midpoint is larger (Auton:2009).

(5) In solution, the N-terminal peptide binds specifically to the truncated A1-1261. This binding is observed in the unfolding transition enthalpy, which changes sigmoidally with respect to the natural log of the peptide concentrations from the enthalpy associated with A1-1261 unfolding to an enthalpy characteristic of A1-1238 unfolding. Scrambling the peptide sequence did not have any change in the unfolding properties of A1-1261 and the N-terminal peptide of correct sequence did not bind to A1-1238. These results indicate site and sequence specific binding of the peptide to A1-1261 at a site that is normally occupied by the covalently linked N-terminal peptide in A1-1238.

In von Willebrand disease, the effect of inherited type 2B and 2M mutations on the N<->I conformational equilibrium is now established for several mutations (Auton:2009). These subtypes of mutations in the A1 domain cause clinically opposite phenotypes resulting in bleeding. From the perspective of reversible urea denaturation at variable temperature, it has been shown that type 2M mutations shift the equilibrium in favor of the native state and decrease the affinity of A1 for platelet GP1bα. Conversely, type 2B mutations shift the equilibrium in the opposite direction toward the intermediate state and increase the affinity of A1 for platelet GP1bα. The inventor studied the irreversible thermal stability of A1 in the absence of urea and have demonstrated that truncating the N-terminal linker peptide from the A1 domain decreases the enthalpy of the N->I transition without altering the transition temperature. This will necessarily increase the rate of unfolding from the native to intermediate state at temperatures below the transition temperature. However, the thermal stability of A1-1261 in solution can be restored to that of A1-1238 through specific binding of the free N-terminal peptide. The rate dependent effect of this intramolecular interaction between the A1 domain and its N-terminal linker establishes the possibility of an on/off conformational switch that is affected by vWD mutations and by rheological shear stress on vWF multimers. In the crystal structure, the most frequently identified type 2B mutations cluster in the loops and turns near the interface between the lower surface of the domain and the N-terminus. In addition to their destabilization of the globular domain structure, part of their effect on vWF conformation could be to favor dissociation of this N-terminal linker resulting in the uncoupling of A and possibly D domains within vWF which would enhance interactions between A1 and platelet GP1bα. The inventor observed that a monospecific antibody (A108), directed against the sequence D₁₄₄₄-E₁₄₅₂ within the α helix of the A1 domain, has a greater reactivity to the truncated 1261-A1A2A3 tridomain relative to the longer 1238-A1A2A3 tridomain. It also has a greater reactivity to purified plasma vWF in the presence of 0.5 mg/mL ristocetin (Auton:2012). Therefore, it is plausible that this structural region of the A1 domain plays a role for the binding of the N-terminal linker.

A corollary to the effect of vWD mutations on A1 domain conformational equilibria is their effect on the shear stress dependent platelet rolling velocities on immobilized A1 and force dependent single bond lifetimes between vWF A1 and platelet GP1bα. These properties are proportional to the strength of the interaction. The inventor previously demonstrated on a single molecule scale that this interaction becomes stronger and increases the lifetime of the protein complex up to a critical threshold force above which the bond weakens and the lifetime decreases (Yago:2008). This force sensing binding process referred to as “catch-bonding” regulates platelet detachment, rolling velocities and overall adhesiveness of vWF as a function of shear stress. Type 2B mutations shift this threshold to lower forces due to the low stability of the native state. Consequently, at lower shear, attached platelets persist due to the higher bond strength and increase the risk for microthrombi. The stabilization of the native state caused by type 2M mutations shifts this threshold to higher forces and weakens the bond strength at physiological shear stress thereby decreasing the adhesive capacity of vWF for platelet GP1bα (Auton:2010).

The inventor shows that the platelet pause time to immobilized A1 has a similar behavior to the single molecule bond lifetime measurements with a sudden increase in platelet pause time at a critical shear threshold ≈1500 s⁻¹ (Yago:2008). Truncation of the N-terminal linker from the A1 domain enhances the pause times at low shear and shear rates that define the catch bond regime. However, addition of saturating amounts of free N-terminal peptide to the surface immobilized A1-1261 restores the pause times to those observed for A1-1238. Considering both of these observations, the fact that the binding of the free peptide in solution to A1 restores both the kinetic thermal stability and the catch-bond properties of the truncated domain to that of the full length A1 domain demonstrates that the binding of this N-terminal linker is specific and critical for modulating the rheological dependence platelet adhesion to vWF.

The inventor's previous studies on the A1A2A3 tridomain fragment of vWF have demonstrated that similar to gain of function mutations (Auton:2010), truncation of this sequence also destabilizes the quaternary association of these domains resulting in effective inhibition of RIPA and fibrinogen-dependent platelet activation under high shear (Auton:2012). These studies confirmed a structural role for this N-terminal sequence to maintain inter-domain associations and keep the tridomain in a thermodynamically stable binding incompetent conformation. The observations reported here highlight another role of the N-terminal linker as a force sensor for the regulation of platelet adhesion to the A1 domain in the open active state of vWF.

In conclusion, the rotational and elongational forces on plasma vWF and the tensile forces on vascular wall tethered ULvWF multimers that occur in the presence of rheological shear likely activate vWF in a two step process that first dissociates the A domains and subsequently modulates the kinetic stability of the A1 domain and its affinity for platelet GP1bα. These processes appear to be regulated by an on/off conformational equilibria between the A1 domain and its N-terminal flanking sequence although this may be a relatively small contribution to the overall process of conformational activation of full length VWF in vivo.

Proteins. Recombinant human vWF A1-1238 (amino acids Q₁₂₃₈-P₁₄₇₁) and its N-terminal truncated variant A1-1261 (amino acids D₁₂₆₁-P₁₄₇₁) were expressed in E. coli M-15 cells as fusion proteins containing a N-terminal 6×His-Tag using BamHI and HindIII restriction sites in the Qiagen pQE-9 plasmid vector (Morales:2006, Cruz:2000). Proteins were purified from inclusion bodies by solubilization in 6 M GuHCl followed by refolding by excessive dilution into 4 L TBS-T and isolated by affinity chromatography using Ni²⁺-chelated Sepharose followed by a second purification via Heparin-Sepharose. The purity of the recombinant proteins was confirmed via reducing SDS-PAGE and also by size exclusion chromatography using a Phenomenex SEC S3000 column on a BioCAD Sprint perfusion chromatography system. Proteins were stored at 0° Celsius in 150 mM NaCl, 25 mM Tris HCl, pH=7.4 and dialyzed overnight against a temperature stable buffer-mixture of 10 mM sodium acetate, 10 mM Na₂HPO₄, 10 mM Glycine, 150 mM NaCl, 1 mM EDTA, pH=8 before all measurements.

The N-terminal peptide (QEPGGLVVPPTDAPVSPTTLYVE (SEQ ID NO.:1)) corresponding to residues Q1238-E1260 and the scrambled peptide (QLPTGVLGEPSDAVPTVYEVTPPG (SEQ ID NO.:2)) were synthesized at the Mayo Clinic Proteomics Core Lab with a free N-terminal amine and C-terminal acid, checked for purity via reversed phase HPLC and their correct masses of 2365.2 Da and 2430.25 Da were verified via electrospray ionization mass spectrometry.

Protein and peptide concentrations were quantified using a Shimadzu UV2101PC spectrophotometer with the Pace method (Pace:1995) from absorption at λ=280 nm minus twice the absorption at λ=333 nm for correction of light scattering. Extinction coefficients for A1-1238 (ε=15350 L/mol/cm) and A1-1261 (ε=14235 L/mol/cm) and the N-terminal peptide (ε=1215 L/mol/cm) were calculated from the number of tyrosines (A1-1238=8, A1-1261=7, N-terminal peptide=1) and a single tryptophan.

Determination of platelet pause times. Determination of platelet pause times A Glycotech† rectangular parallel-plate flow chamber was mounted to slides with a chelated Cu 21 surface obtained from Microsurfaces Inc. # to which the A1-1261 and A1-1238 domains in TBS were immobilized by the 63His-Tag. Citrated whole blood (1 mL) obtained from informed consent of healthy donors with approval from the Baylor College of Medicine institutional review board followed by buffer was perfused through the chamber at 300s21 for A1-1261 and 700s21 for A1-1238 shear rate to attach platelets to the surface immobilized proteins using a syringe driven variable flow rate pump. The shear was increased incrementally and 1 min movies were recorded at 25 frames/s in phase contrast with 232 pixel binning on a Zeiss Axiocam MRm camera attached to a Zeiss Axio Observer D1 inverted microscope. Tracking analysis was performed using Mediacybernetics ImagePro Premier§. The resulting X-Y coordinate data for each platelet track was differentiated into instantaneous velocities using a Savitzky-Golay algorithm (Savitzky:1964) with a five data point window size and a second-order polynomial implemented into a Mathamatica notebook. These platelet tracks were retained for statistical analysis if the platelet was present for at least 1 s and travelled a total distance greater than 1 lm. Distance travelled in pixels was converted to lm given the camera pixel size=6.45 μm², pixel binning, and microscope magnification=400×. Pause times were determined by the amount of time (seconds) a platelets velocity was =0±0.13 μm/s, within the noise. Pause times for all platelet tracks are reported as the mean of the medians. Examples of this analysis for individual platelets and for multiple platelets are provided in the Supporting Information.

Protein denaturation. Urea and thermal unfolding of A1-1238 and A1-1261 was monitored by CD and fluorescence spectroscopy and DSC using an Aviv Biomedical Model 420C CD spectrometer, a Horiba Jobin-Yvon Fluorolog 3 spectrofluorimeter equipped with a Wavelength Electronics Model LF1-3751 temperature controller and a TA Instruments NanoDSC.

Isothermal urea induced unfolding of A1-1261 and A1-1238 at 25° Celsius was monitored via CD at λ=222 nm using a quartz cuvette with a path length of 1 mm and a protein concentration of 10 μM after an overnight equilibration. CD-signal was averaged for 10 min, corrected for the corresponding CD-signal of the buffer and converted into mean molar ellipticities per amino acid residue (θ_{MRW}).

Prior to all spectroscopic thermal scans, protein samples were equilibrated at 10° Celsius for 15 min to obtain a stable baseline. CD thermal scans between 10 and 95° Celsius were recorded at λ=222 nm using a protein concentration of 1 or 2 μM in a 1 cm quartz cell under moderate stirring. Scan rates were 0.5, 1.0, 1.5 and 2.0° Celsius/min. Integration time for each data point was 20 s with a 1 nm bandwidth. Fluorescence thermal scans between 10 and 95 Celsius were recorded at an emission λ=359 nm after excitation λ=280 nm or λ=295 nm using 2 μM protein in a 1 cm quartz cell with moderate stirring. Scan rates were 0.4 or 0.6, 0.9, 1.6 and 2.0° Celsius/min. At each temperature, relative fluorescence intensity was collected for 4 s and averaged. Thermal scans in the presence of N-terminal peptide at 2.0° Celsius/min were preceded by 1 h incubations at 20° Celsius with 0.02-300 μM peptide.

DSC was performed at 3 atm pressure. Protein samples (40 to 50 μM) and buffers were degassed under moderate stirring prior to use. The calorimeter was equilibrated overnight with buffer in the sample and reference cell to obtain a stable baseline and the protein sample was loaded during an equilibration step between scans. One measurement per second was recorded at 2.0° Celsius/min. DSC-traces were background corrected with the following irreversible scan that was used as a baseline. The molar heat capacity was calculated from the calorimetric power compensation (μJ/s) using the relation, C_{P} (kJ/mol/K)=Power (μJ/s)/[(v (Celsius/min))/60*V (mL)*c (μmol/L)]. The excess molar heat capacity, <C_{P}> (kJ/mol/K) was obtained by subtracting a polynomial baseline fit to the pre- and post-transition regions of the heat capacity traces using a Mathematica\footnote (www.wolfram.com/mathematica) notebook.

Isothermal urea unfolding was analyzed using a three state reversible model (N<->I<->D) as previously described (Auton:2009, Auton:2007). All thermal unfolding transitions were analyzed according to the irreversible models provided in the Supporting Information. (Atkins:1994, Lyubarev:1998, Kurganov:1997, Rainville).

Frame by frame platelet trajectory analysis. Tracking coordinates of individual platelets in units of pixels were exported from ImagePro Premier as a text file and imported into a Mathematica notebook that calculates a Savitzky-Golay first derivative of the total distance traveled over time (Savitzky:1964). The distance was calculated from the X and Y coordinates with the following equation.

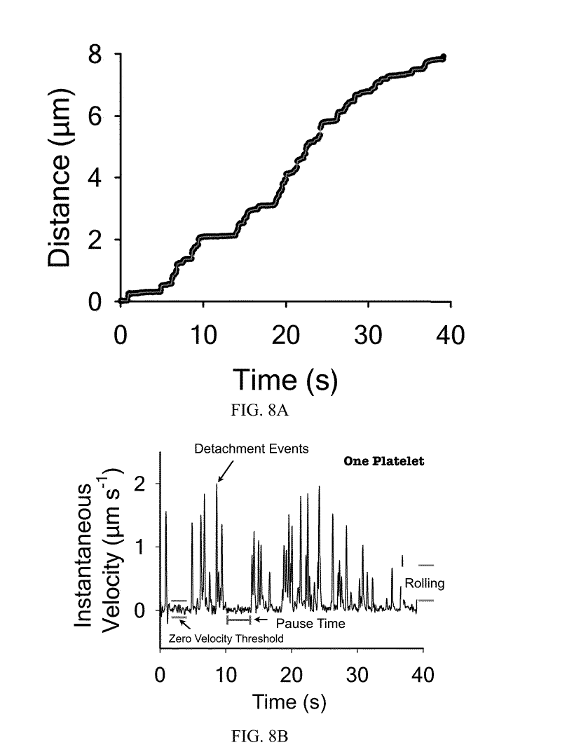

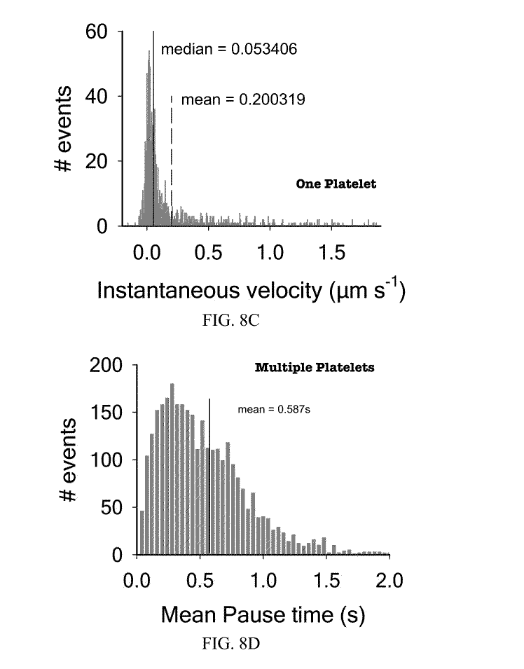

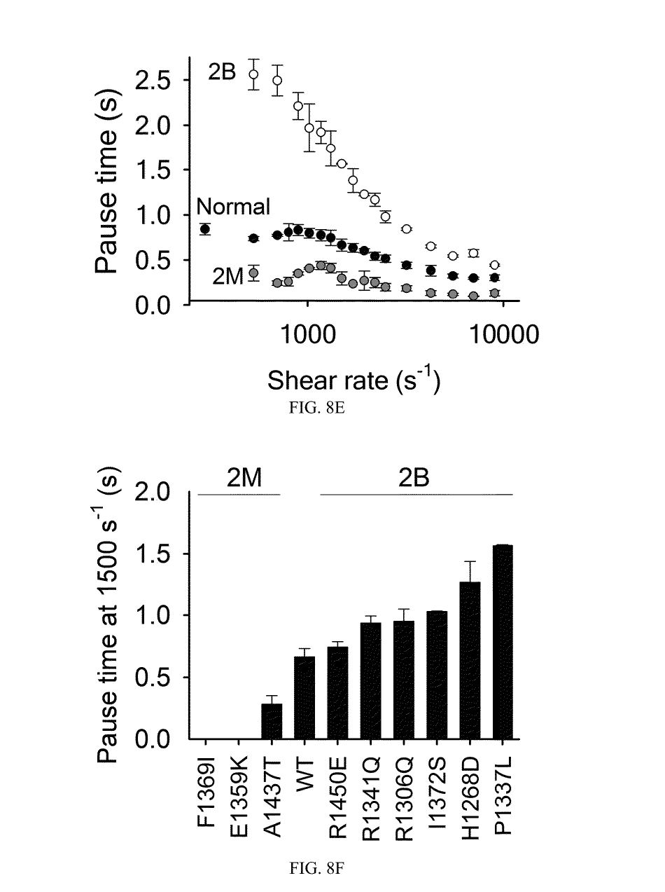

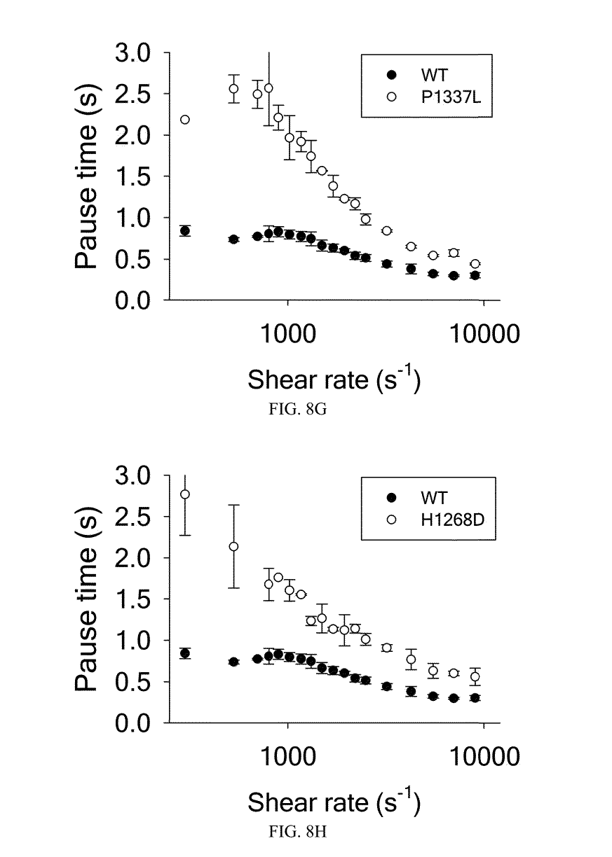

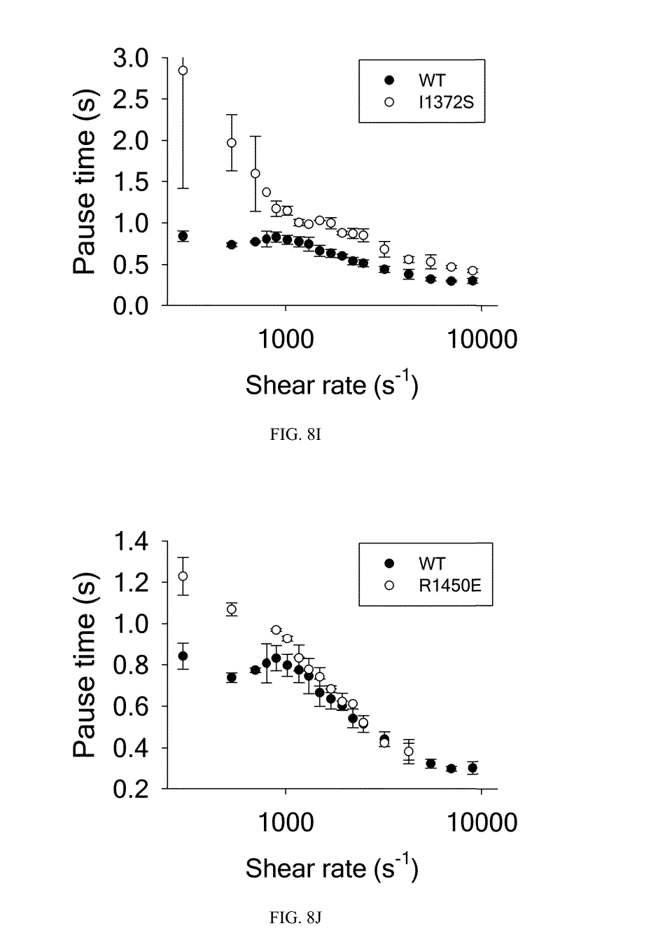

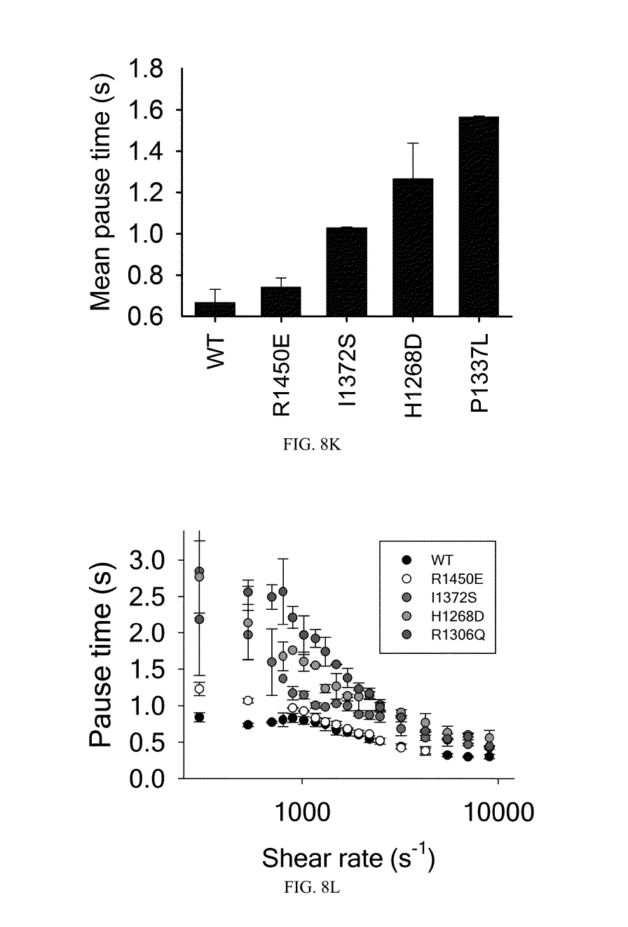

D(time)=\sqrt{(xₜ−x₀)²+(yₜ−y₀)²} (1)

where x₀ and y₀ are the coordinates of a platelet on the first frame of the movie and xₜ and tₜ are the coordinates on subsequent frames of the movie. The framerate was 24 frames/sec. FIG. 8A illustrates representative data for a single platelet track. The Savitzky-Golay first derivative gives the instantaneous velocity of a platelet over time (FIG. 8B). These velocities were translated into histograms to determine and illustrate the mean and median velocities obtained for a single platelet.

FIG. 8C shows the set-up of the present invention, in which a parallel plate flow chamber was used to examine the physics of shear-induced adhesion, in this example, of platelets. The top left shows a schematic of the surface of the plate onto which a copper containing linking agent is used to trap a protein with a 6×His tag and to which, in this example, a platelet binds. In the top right, an example of the microfluidic chamber is shown. In the bottom left, the platelets that adhere to the surface have been imaged, and in the bottom right, the trajectories of each of the platelets imaged in the bottom left are individually tracked over time. FIG. 8D shows that pause times are determined by the amount of time a platelet's velocity is statistically zero within a noise threshold of 0±0.25 μm/s and all pause time event are averaged over all platelets analyzed.

Quantitative distinction between type 2B and type 2M VWD mutational effects on platelet adhesion. The inventor obtained data on a number of gain (2B) and loss (2M) of function mutations in the single A1 domain. FIG. 8F shows a rank order of mean pause times for platelets adhering to type 2B and 2M mutated A1 domain relative to WT A1 domain at 1500 s⁻¹. FIG. 8G shows a graph that distinguishes the mean pause times of wild type platelet binding versus a vWD P1337L mutant versus shear rates. FIG. 8H shows a graph that distinguishes the mean pause times of wild type platelet binding versus a vWD H1268D mutant versus shear rates. FIG. 8I shows a graph that distinguishes the mean pause times of wild type platelet binding versus a vWD I1372S mutant versus shear rates. FIG. 8J shows a graph that distinguishes the mean pause times of wild type platelet binding versus a vWD R1450E mutant versus shear rates. FIG. 8K shows a histogram that distinguishes the mean pause times of wild type platelet binding versus vWD R1450E, I1372S, H1268D, and P1337L mutants versus shear rates. FIG. 8L shows a graph that distinguishes the mean pause times of wild type platelet binding versus vWD R1450E, I1372S, H1268D, and P1337L mutants versus shear rates. Compares the shear rate dependence of platelet pause times on a mild loss-of-function type 2M mutation (A1437T) and an extreme type 2B gain-of-function mutation (P1337L) with normal wild-type A1 domain. The platelet pause times associated with these mutations represent the breadth of our dynamic range. FIG. 8F illustrates a rank order of pause times at 1500 s⁻¹ from complete loss-of-function in the case of type 2M mutations F1369I and E1359K to a range of pause times obtained for gain-of-function type 2B mutations. These results demonstrate that the method can discriminate not only between gain- and loss-of-function but also various degrees of function that define the strength of platelet adhesion to these vWF A1 domain variants.

Since the velocity distribution was always skewed to high velocities the median velocity was taken as the most representative description of the most probable velocities. All distances and velocities were converted from pixels to micrometers by multiplying by the following factor.

Conversion=PS*PB/M=0.03225 μm/pixel (2)

Where the camera pixel size PS=6.45 μm on edge, the camera pixel binning PB=2 and the microscope magnification M=400.

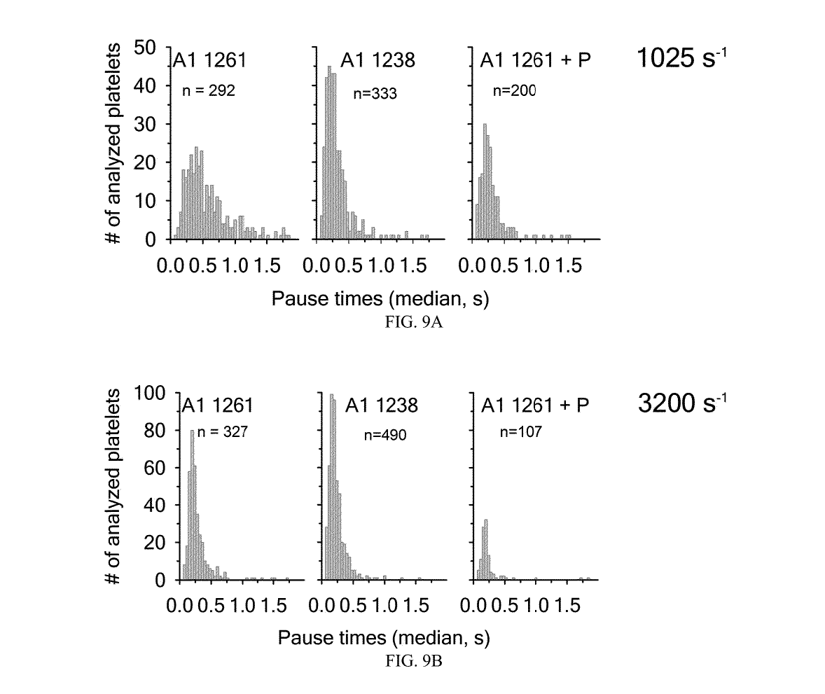

Pause times were determined from the length of time the platelet velocity remained within 0±4 pixel/sec=0.129 μm/sec as illustrated in FIG. 8B. Median pause times were calculated from velocity time profiles for all platelet tracks, stored in a list and translated to histograms for multiple platelets. The mean of the median pause times was determined to be representative of the most probable pause times. FIGS. 9A and 9B give representative pause time histograms for platelets translocating on surface immobilized A1-1261, A1-1238, and A1-1261 in the presence of peptide at two shear rates, 1025 s⁻¹ at the beginning of the catch bond phase and at 3200 s⁻¹ well into the slip bond phase. See also FIG. 1.

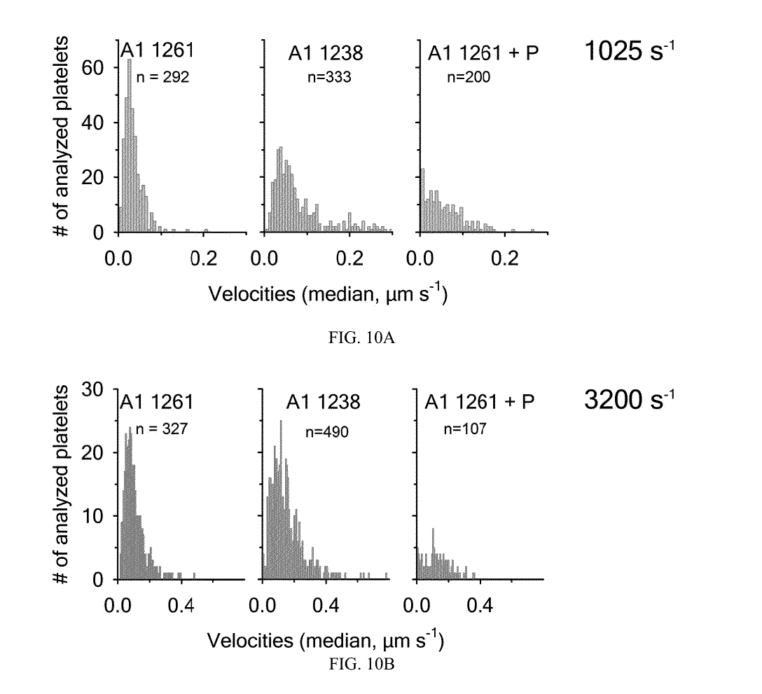

The median velocities travelled by all platelets were also translated to histograms for multiple platelets. The mean of the median velocities was determined to be representative of the most probable velocities. FIGS. 10A and 10B give representative velocity histograms for platelets translocating on surface immobilized A1-1261, A1-1238, and A1-1261 in the presence of peptide at two shear rates, 1025 s⁻¹ at the beginning of the catch bond phase and at 3200 s⁻¹ well into the slip bond phase. See also the inset of FIG. 1.

Analysis and Fitting of Two-State and Three-State Thermal Transitions

For an irreversible two-state transition, unfolding is kinetically controlled by a first order rate constant that varies with temperature according to the Arrhenius equation (Atkins:1994). In our analysis the inventor refers to the intermediate state as the final irreversible state for all thermal transitions involving the unfolding of A1-1261 in the absence of peptide when monitored by fluoresence, CD and DSC and for A1-1238 when monitored by CD and DSC. The following equations derived below are equally valid for any two state irreversible transitions The activation energy in going from N->I is ΔG^{‡}=−RT ln k^{‡} and the inventor expands ln k^{‡} as a function of temperature in terms of Δβ=(β−β*)=(1/RT−1/RT*) using a Taylor series, where T̂* is the temperature at which ΔG^{‡}=0.

$$ln {k}^{‡} ( T ) = ln {k}_{T *}^{†} + {Δβ ( \frac{\partial ln {k}^{‡}}{\partial β} )}_{T *} + \frac{{Δβ}^{2}}{2} {( \frac{{\partial }^{2} ln {k}^{‡}}{\partial {β}^{2}} )}_{T *} \qquad ( 3 )$$

where ln k_{T*}^{‡}=0, (θ ln k^{‡}/∂β)_{T*}=−ΔH_{T*}^{‡} corresponds to the enthalpy of activation and (∂² ln k^{‡}/∂β²)_{T*}=RT*²ΔC_{PT*}^{‡} is the relationship that defines the heat capacity of activation. The inclusion of the second order parameter in this Taylor expansion is given only for the sake of completion and in all cases, the fitting of the data presented herein did not justify a non-zero heat capacity term.

The rate equation for the irreversible formation of I with respect to \β is in terms of the population, P_{I}.

$$\frac{\partial {P}_{I} ( T )}{\partial β} = - \frac{\partial {P}_{N} ( T )}{\partial β} = \frac{\partial {P}_{I}}{\partial t} \frac{\partial t}{\partial T} \frac{\partial T}{\partial β} = \frac{- {RT}^{2} {k}^{‡} ( T )}{v} ( 1 - {P}_{I} ( T ) ) \qquad ( 4 )$$

where ∂P_{I}/∂t=k(1−P_{I}), ∂t/∂T is the inverse of the thermal scan rate) (v) and ∂T/∂β=−RT². Separation of variables followed by numerical integration results in the following expression for the intermediate state population, where (P_{N}+P_{I}=1).

$${P}_{I} ( T ) = 1 - exp ( \frac{1}{R v} \int \frac{{k}^{‡} ( T )}{{β}^{2}} \partial β ) \qquad ( 5 )$$

For spectroscopy the inventor used the following function for the observed CD or fluorescence change.

Obs(T)=Obs_{N}(T)(1−P_{I}(T))+Obs_{I}(T)P_{I}(T) (6)

where Obs_{N}(T) and Obs_{I}(T) are linear baseline functions for the native and intermediate state observables.

In calorimetry, the observable is excess heat capacity [symbol] C_{P} [symbol] (T) and it is derived through differentiation of the excess enthalpy [symbol] H [symbol] (T)=P_{I}(T)ΔH^{‡}(T) with respect to β, where ΔH^{‡}(T)=ΔH_{T*}^{‡}−ΔβRT*²ΔC_{PT*}^{‡}.

$$〈 {C}_{P} 〉 ( T ) = \frac{- 1}{{RT}^{2}} \frac{\partial 〈 H 〉 ( T )}{\partial β} = \frac{- 1}{{RT}^{2}} ( {P}_{I} ( T ) \frac{\partial Δ {H}^{‡} ( T )}{\partial β} + Δ {H}^{‡} ( T ) \frac{\partial {P}_{I} ( T )}{\partial β} ) \qquad ( 7 )$$

In the limit of ΔC_{PT*}^{‡}→0, equation 7 reduces to after substitution of equations 4 and 5.

$$〈 {C}_{P} 〉 ( T ) = \frac{Δ {H}_{T *}^{‡} {k}^{‡} ( T )}{v} exp ( \frac{1}{Rv} \int \frac{{k}^{‡} ( T )}{{β}^{2}} \partial β ) \qquad ( 8 )$$

Because the fluorescence thermal scans of A1-1238 and A1-1261 in the presence of the N-terminal peptide showed a scan rate dependent second transition at higher temperatures that represented dissociation of the peptide from A1 domain, the inventor used a three-state irreversible model accounting for two irreversible rate constants that describe the N->I->D reaction (Lyubarev:1998).

ln k₁^{‡}(T)=ln k_{1,T*}^{‡}−ΔβΔH_{1,T*}^{‡} (9)

ln k₂^{‡}(T)=ln k_{2,T*}^{‡}−ΔβΔH_{2,T*}^{‡} (10)

where the inventor assumed a net zero heat capacity of activation for simplicity. The rate equations for the irreversible formation of I and D and the disappearance of N with respect to \β is in terms of their populations

$$\begin{matrix}\frac{\partial {P}_{N} ( T )}{\partial β} = \frac{{RT}^{2} {k}_{1}^{‡} ( T )}{v} {P}_{N} ( T ) & ( 11 ) \\ \frac{\partial {P}_{I} ( T )}{\partial β} = \frac{- {RT}^{2}}{v} ( {k}_{1}^{‡} ( T ) {P}_{N} ( T ) - {k}_{2}^{‡} ( T ) {P}_{I} ( T ) ) & ( 12 ) \\ \frac{\partial {P}_{D} ( T )}{\partial β} = \frac{- {RT}^{2} {k}_{2}^{‡} ( T )}{v} {P}_{I} ( T ) & ( 13 )\end{matrix}$$

Equation 11 is solved as in the case of a two-state model above.

$${P}_{N} ( T ) = exp ( \frac{1}{Rv} \int \frac{{k}_{1}^{‡} ( T )}{{β}^{2}} \partial β ) \qquad ( 14 )$$

Rearranging equation 12 reveals a first order linear differential equation in the following form (Rainville)

$$\frac{\partial {P}_{I} ( T )}{\partial β} - \frac{{k}_{2}^{‡} ( T ) {P}_{I} ( T )}{Rv {β}^{2}} = - \frac{{k}_{1}^{‡} ( T ) {P}_{N} ( T )}{Rv {β}^{2}} \qquad ( 15 )$$

that solves to equation 16 after substituting in equation 14 for the population of the native state.

$${P}_{I} ( T ) = \frac{- 1}{Rv} exp ( \frac{1}{R v} \int \frac{{k}_{2}^{‡} ( T )}{{β}^{2}} \partial β ) \int \frac{{k}_{1}^{‡} ( T )}{{β}^{2}} exp ( \frac{1}{R v} \int \frac{{k}_{1}^{‡} ( T ) - {k}_{2}^{‡} ( T )}{{β}^{2}} \partial β ) \partial β \qquad ( 16 )$$

The population of the denatured state is simply the following difference with substitution of equations 14 and 16.

P_{D}(T)=1−P_{N}(T)−P_{I}(T) (17)

The following function for the observed fluorescence change was used.

Obs(T)=Obs_{N}(T)P_{N}(T)+Obs_{I}(T)P_{I}(T)+Obs_{D}(T)P_{D}(T) (18)

where Obs_{N}(T), Obs_{I}(T) and Obs_{D}(T) are linear baseline functions for the native, intermediate and denatured state observables.

Equations 6, 8, and 18 were used to fit all thermal transitions using a Microsoft Excel script (written in our laboratory) that incorporates Boole's method for the numerical integration (en.wikipedia.org/wiki/Boole's\ rule). Fitting results were minimized to the total standard deviation of the sum of squared residuals. The goodness of fit was calculated via the R² coefficient of determination from the calculated total sum of squares SSₜₒₜ=Σᵢ(yᵢ− y̅)², regression sum of squares SS_{reg}=Σᵢ(fᵢ− y̅)², and the residual sum of squares SSₑᵣᵣ=Σᵢ(yᵢ−fᵢ)², where yᵢ and fᵢ are individual observed and fit values at any given temperature and y̅=(Σᵢ₌₁ⁿ yᵢ)/n is the mean experimental observable for the entire transition.

$${R}^{2} = 1 - \frac{{S}_{err}}{{S}_{tot}} = 1 - \frac{{{Σ}_{i} ( {y}_{i} - {f}_{i} )}^{2}}{{{Σ}_{i} ( {y}_{i} - \tilde{y} )}^{2}} \qquad ( 19 )$$

Analysis and fitting of the change in thermal unfolding enthalpy upon the binding N-terminal peptide to A1-1261 in solution.

For the thermal unfolding studies in the presence of peptide, the inventor treated the change in enthalpy of unfolding, ΔH_{T*}^{‡} as an observable directly proportional to the degree of binding, X, because the T* was not dependent on peptide concentration.

ΔH_{T*}^{‡}=ΔH_{T*,A1-1261}^{‡}+(ΔH_{T*,A1-1238}^{‡}−ΔH_{T*,A1-1261}^{‡})X (20)

For the simple single site binding model, A1+P<->A1P, where P is the peptide, the degree of binding is given by

$$X = \frac{K [ P ]}{1 + K [ P ]} \qquad ( 21 )$$

where K is the binding affinity and the free peptide, [P], concentration is given in terms of the total protein, A1_{T}, and peptide, P_{T}, concentrations.

$$[ P ] = \frac{- 1 + K ( {P}_{T} - A {1}_{T} ) + \sqrt{4 {KP}_{T} + {( - 1 + K ( {P}_{T} - A {1}_{T} ) )}^{2}}}{2 K} \qquad ( 22 )$$

### Example 2

### A Molten Globule Intermediate of the Von Willebrand Factor A1 Domain Firmly Tethers Platelets Under Shear Flow

Clinical mutations in patients diagnosed with Type 2A von Willebrand disease (vWD) have been identified that break the single disulfide bond linking N- and C-termini in the vWF A1 domain.

The inventor modeled the effect of these mutations on the disulfide-bonded structure of A1 by reducing and carboxy-amidating these cysteines. Solution biophysical studies show that loss of this disulfide bond induces a molten globule conformational state lacking global tertiary structure but retaining residual secondary structure. The conformational dependence of platelet adhesion to these native and molten globule states of A1 is quantitatively compared using real-time high-speed video microscopy analysis of platelet translocation dynamics under shear flow in a parallel plate micro-fluidic flow chamber. While normal platelets translocating on surface-captured native A1 domain retain the catch-bond character of pause times that increase as a function of shear rate at low shear and decrease as a function of shear rate at high shear, platelets that interact with A1 lacking the disulfide bond remain stably attached and do not translocate. Based on these findings, it was found that the shear stress-sensitive regulation of the A1-GPIb interaction is due to folding the tertiary structure of this domain. Removal of the tertiary structure by disrupting the disulfide bond destroys this regulatory mechanism resulting in high-strength interactions between platelets and vWF A1 that are dependent only on residual secondary structure elements present in the molten globule conformation.

Type 2A von Willebrand disease is clinically identified by a loss of high and intermediate von Willebrand factor multimers resulting in a loss of platelet-dependent function. The majority of missense mutations that cause type 2A disease occur in the A2 domain and are grouped by their effects on cellular retention of vWF or the susceptibility of vWF to proteolysis by ADAMTS13. However, a few mutations also classified as 2A occur in the A1 domain. The most puzzling are those that abolish the single disulfide bond in the A1 domain, which is required to maintain the native structure of the domain. These point mutations identified in vWD patients result in the substitution of Cysteine 1272 for Arginine, Glycine, Phenylalanine and Serine and Cysteine 1458 for Tyrosine (Woods:2012, Penas:2004, Meyer:1997, Lavergne:1992) Patients with C1272S and C1272F substitutions have been reported to be heterozygous so that one of the alleles is normal and although not reported, it is likely that the other known mutations are also heterozygous in vWD patients (Woods:2012, Penas:2004) Phenotypically, these patients display the distinctive clinical features of abnormal bleeding, decreased vWF ristocetin cofactor activity relative to vWF antigen, and diminished ristocetin-induced platelet agglutination (RIPA). Platelet counts are generally normal, but thrombocytopenia can occur (Meyer:1997).

Early studies resulted in disparate conclusions regarding the effect of the C1272-C1458 disulfide bond on the structure and function of the A1 domain. Initially, two independent studies using recombinant A1 domain demonstrated 1) a requirement of the disulfide bond for effective inhibition of RIPA using fixed platelets in the presence of purified plasma vWF and 2) the aggregation of washed platelets in the absence of vWF induced by the recombinant domain (Azuma:1993, Cruz:1993). However, a subsequent report demonstrated that a reduced and alkylated dispase-digested fragment of vWF containing residues V1262-K1491 of the A1 domain displayed dose-dependent inhibition of shear-induced platelet agglutination (SIPA) and was more effective at inhibiting the binding of purified vWF and LJ-Ib1 (an antibody against GPIbα) to GPIbα than the disulfide-intact fragment (Andrews:1989, Miura:1994) To further complicate the issue, studies on recombinant multimeric vWF containing mutations C1272R, C1272G and C1458G illustrated that binding of vWF to fixed platelets was dependent on the specific cysteine that was mutated (Siguret:1996). Mutating C1272 with either Arginine or Glycine caused a spontaneous interaction with platelets whereas C1458G resulted in a complete loss of function. Multimer gels also showed low to intermediate molecular weight multimers for C1272R and C1272G and a complete loss of all multimeric species for C1458G.

Studies from the Ruggeri lab illustrated an increase in GPIbα binding affinity of 1-2 orders of magnitude upon reducing and alkylating the disulfide bond. On the basis of fluorescence and circular dichroism, this result was interpreted to be a “loosening of native tertiary structure” (Miyata:1996) Furthermore, it was later demonstrated in flow chamber studies that a greater number of platelets adhered to glass surface-immobilized reduced/alkylated A1 domain at low shear than for disulfide-intact A1. At high shear this observation was reversed suggesting that fluid shear in laminar flow could switch the conformation of A1 from a low to a high affinity state that was similar to reduced/alkylated A1 with respect to platelet adhesion at low shear (Miyata:1999) These two key observations combined with our identification of a thermodynamically stable intermediate conformation in the three-state unfolding pathway of the A1 domain led to current understanding of how gain and loss of function mutations in the A1 domain affect the linkage between conformation and GPIbα binding affinity and the effect of shear stress on this linkage (Auton:2010, Auton:2009, Auton:2007tl) This mechanism appears to involve a delicate balance in the thermodynamic equilibrium between the low affinity native and high affinity intermediate state resulting in differential rates of dissociation of GPIbα from the A1 domain that modulate the efficiency of platelet adherence to vWF as a function of shear stress (Auton:2010, Kim:2010). Type 2 mutations affect the efficacy of platelet adherence to vWF and change the affinity of GPIbα binding by altering this conformational equilibrium in favor of one or the other thermodynamic state (Auton:2010, Auton:2009).

In principle, type 2A mutations that break the disulfide bond in A1 could cause severe problems in hemostasis that lead to microvasular thrombosis because of the enhanced affinity of this intermediate conformation for platelet GPIbα. Here, the inventor provides a rigorous functional and biophysical comparison of these two thermodynamic conformational states using native disulfide-intact A1 and reduced and carboxyamidated (RCAM) A1 as a model of the intermediate state (Auton:2010). Using a parallel-plate micro-fluidic flow chamber that immobilizes the A1 domains via an N-terminally fused 6× Histidine-Tag engineered into their sequence, the inventor found that disulfide-intact A1 exhibits catch-bond characteristics in the platelet pause times with a biexponential survival function that quantifies a unique shear dependence of the dissociation rate constants. In contrast, the enhanced affinity of RCAM A1 for platelet GPIbα abolishes platelet rolling on surface immobilized RCAM A1 resulting in net zero translocation velocities under shear flow. Biophysical, thermodynamic and spectroscopic comparisons between A1 and RCAM A1 illustrate that the intermediate conformation exists as a molten globule state that lacks tertiary structure while retaining residual secondary structure. This observation implies that folding inhibits the binding potential of A1 and that stable platelet attachment is primarily dependent upon secondary structure elements that are retained in the high affinity intermediate conformation. The implications of these findings pertaining to type 2A von Willebrand disease, vWF multimerization and protective mechanisms for vWF clearance are discussed.

Platelet Adhesion to Surface Captured Disulfide-Intact A1 and RCAM A1 Under Shear Flow.

The interaction of disulfide-intact A1 and of RCAM A1 with platelets was studied by immobilizing the proteins via the 6×His-Tag on Cellix biochips in which the internal channel surface was coated with a Cu2+ chelating chemistry. A 5 μM concentration of protein was immobilized in the biochip and citrated whole blood was perfused at a shear rate of 800 s−1 (FIG. 11B). This perfusion was followed by TBS buffer and after red blood cells cleared the channel, the shear was decremented to 100 s−1 or incremented to 9000 s−1 every two minutes at logarithmic intervals. Surface bound platelets were allowed to equilibrate at a given shear rate for 50 s and a 60 s video at 24 frames per second was recorded and analyzed as described in the methods. The internal channel surface lacking immobilized A1 domain was completely inert to platelets.

At each shear rate, platelet mean pause times and mean translocation velocities were calculated from the X-Y trajectories of moving platelets as described in the methods. Approximately 1000 platelet trajectories were analyzed at each shear rate (FIGS. 11B to 11D). The mean pause times were also calculated from the pause time survival fraction obtained from the cumulative integral of the pause time distribution (FIG. 11B) (Thomas:2006). The survival fraction exponentially decreases with faster decay rates as shear increases. Fitting the survival fraction with a bi-exponential decay function results in the apparent rate constants and amplitudes as a function of shear rate (FIG. 11C). The mean pause times were calculated from the survival fraction using equation 23 and were found to agree with taking the statistical mean of all platelet pause time distributions (FIG. 11A).

$$〈 r 〉 = \frac{{A}_{1}}{{k}_{1}} + \frac{{A}_{2}}{{k}_{2}} \qquad ( 23 )$$

Mean platelet pause times increased from ≅0.75 s at a shear rate of 530 s−1 to ≅0.9 s at 1025 s−1 and then decreased again upon further increase of the shear rate. The mean platelet translocation velocities mirrored the pause times and were minimal at a shear rate of 1025 s−1 and increased at lower and higher shear rates. While performing the flow studies, it was observed that platelets began to detach from surface immobilized disulfide-intact A1 at shear rates ≦300 s−1.

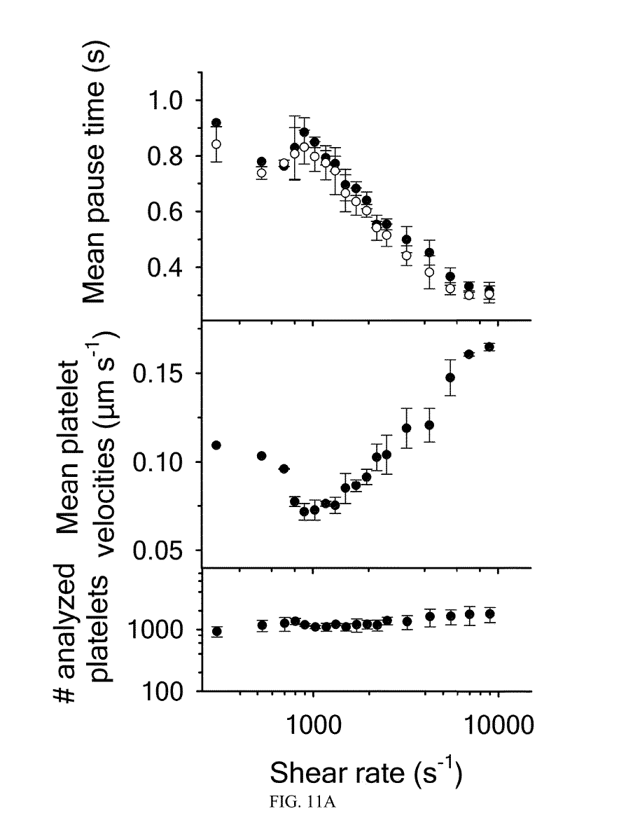

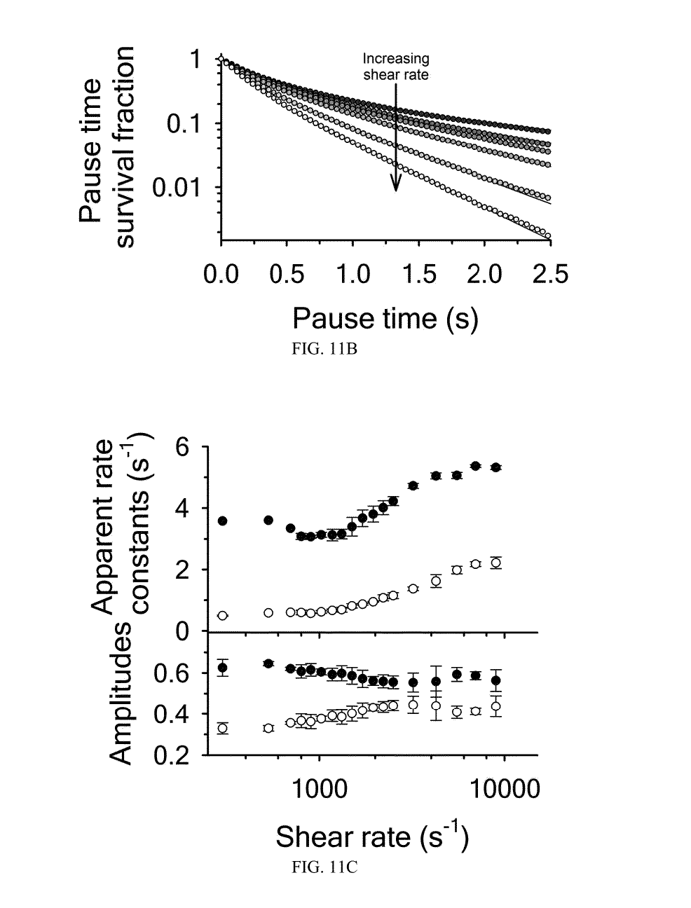

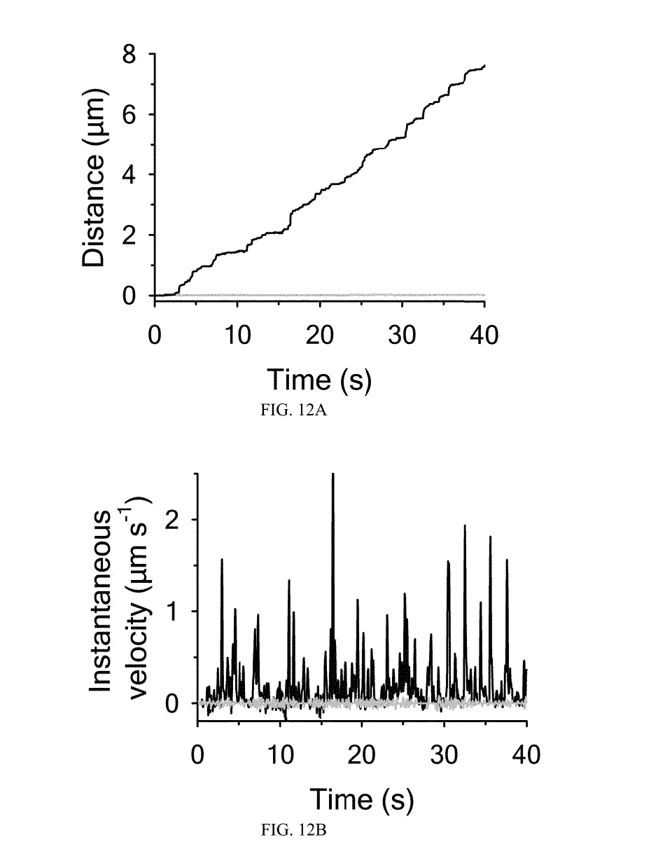

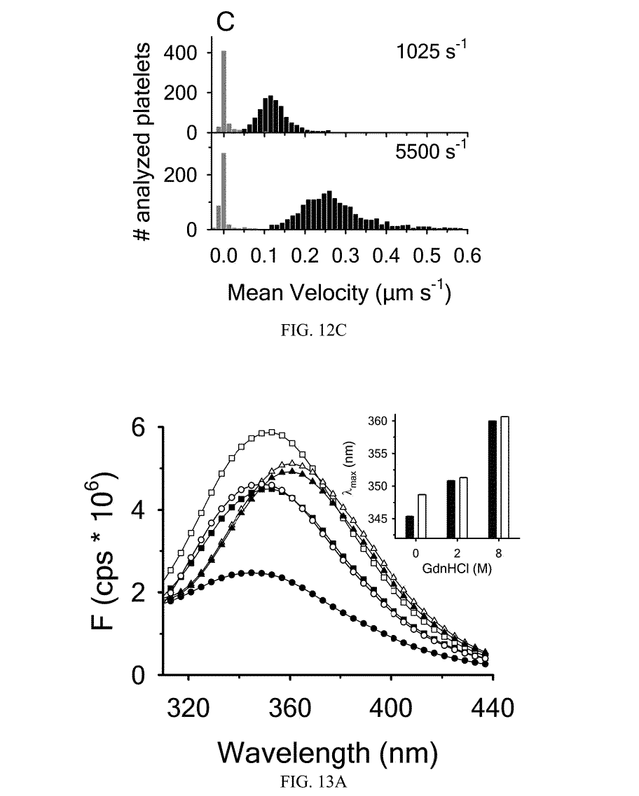

In contrast to disulfide-intact A1, the interactions between surface-immobilized RCAM A1 and platelet GPIbα resulted in captured platelets that did not translocate across the surface at all applied shear rates. Pause time analysis was not possible to do for RCAM A1 because the maximum was limited by the length of the recorded movies. However, the inventor compared the distance traveled and instantaneous velocities. FIG. 12A illustrates the traveled distance for a single platelet translocating on disulfide-intact A1 with a platelet on RCAM A1 over a 40 s timeframe. The traveled distance was converted into instantaneous velocities in FIG. 12B. While the interaction of a platelet with A1 caused many translocation events yielding nonzero velocities, the velocity of a platelet translocating on RCAM A1 was statistically zero. This is also shown by the histograms given in FIG. 12C which report velocity distributions of all analyzed platelet translocations at shear rates of 1025 and 5500 s−1. Furthermore, platelets remained firmly adhered to RCAM A1 at all shear rates investigated up to 9000 s−1 and remained attached to RCAM A1 even after removal of shear stress by stopping the flow. However, the total number of platelets interacting with RCAM A1 was less, by approximately half, than with disulfide-intact A1 indicating that the on-rate for bond formation is reduced for RCAM A1 relative to disulfide-intact A1. Representative movies of platelets translocating on disulfide-intact A1 and RCAM A1 at 1025 s−1 are provided in the supporting information.

Spectroscopic Properties of A1 and RCAM A1.

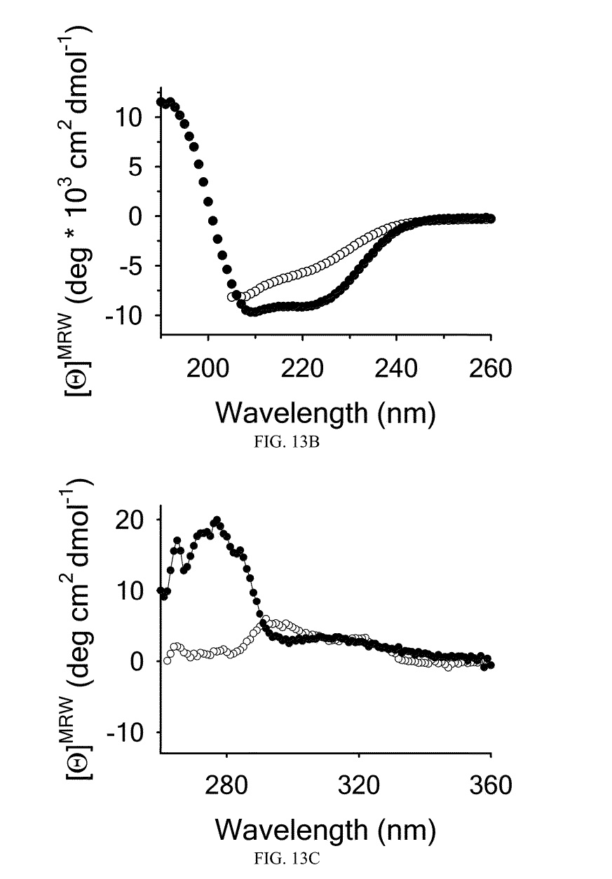

Fluorescence and circular dichroism spectra of A1 and of RCAM A1 were measured to compare the basic spectroscopic properties of the two protein variants (FIG. 13). The fluorescence spectrum of native A1 has a relatively low intensity with λmax of ˜345 nm. In presence of 2 M GdnHCl the fluorescence intensity increases as the interior of the protein becomes solvent accessible and a red shift of the spectrum to ˜351 nm occurs. 8 M GdnHCl decreased the fluorescence intensity and resulted in an additional redshift to ˜360 nm. The fluorescence spectrum of RCAM A1 in presence of 0.5 M GdnHCl showed increased fluorescence intensity and a slightly more red shifted λmax in comparison to A1. Increasing the GdnHCl concentration to 2 M and 8 M resulted in an increased intensity and a λmax comparable to that of the disulfide-intact A1 domain. Far UV CD spectra of the A1 domain are dominated by a high α-helical content whereas RCAM A1 had a reduced α-helical content. Nevertheless, RCAM A1 is still retains significant secondary structure content in solution; the distribution of which is summarized in Table 1. In the near UV, the CD of disulfide-intact A1 show a well-defined spectral banding pattern between 260 and 300 nm indicating significant tertiary structure content that is absent in RCAM A1.

TABLE 1
Secondary structure contribution calculated from CD-spectra*

| Protein | % α-Helix | % β-sheet | % Remainder** |
|---|---|---|---|
| A1 | 38.0 | 15.6 | 46.4 |
| RCAM A1 | 25 | 22 | 53 |

*Contributions were calculated from the CD-spectra using the program CDNN [1].
**Remaining structure contains contributions of β-turns and random coil structure.

Chemical Denaturation of A1 and RCAM A1.

The thermodynamic stability of A1 and of RCAM A1 was determined from GdnHCl and urea induced unfolding measured via CD and FL at 25° Celsius (FIG. 14). Three states, native, intermediate and unfolded, were observed by CD for disulfide-intact A1 in GdnHCl, but urea did not completely unfold the intermediate state. In contrast, the ellipticity for RCAM A1 in the absence of denaturant is comparable to that of the A1 domain in the intermediate state. Complete unfolding of A1 and of RCAM A1 occurs in GdnHCl but not in urea. However, in presence of urea, a small expansion of RCAM A1 compared to A1 in its intermediate state is observed. The obtained parameters for the transitions are summarized in Table 2 below.

TABLE 2
Thermodynamics of GdnHCl- and urea-induced unfolding at
25° C. with circular dichroism as the observable.

| | | A1 | | RCAM A1 | |
|---|---|---|---|---|---|
| Transition | Parameter | GdnHCl | Urea | GdnHCl | Urea |
| N ⇄ I | ΔG⁰ (kJ/mol) | 15.29 ± 0.63 | 13.40 ± 0.49 | ND | ND |
| | m-value (kJ/mol/M) | −18.67 ± 0.76 | −4.70 ± 0.17 | ND | ND |
| | cₘ (M) | 0.819 ± 0.006 | 2.85 ± 0.02 | ND | ND |
| I ⇄ D | ΔG⁰ (kJ/mol) | 29.30 ± 1.32 | 33.6 ± 6.25 | 28.69 ± 1.89 | ND |
| | m-value (kJ/mol/M) | −6.66 ± 0.30 | −3.35 ± 0.62 | −7.49 ± 0.49 | ND |
| | cₘ (M) | 4.40 ± 0.02 | 10.03 ± 0.19 | 3.83 ± 0.03 | ND |

Thermal Denaturation of A1 and of RCAM A1.

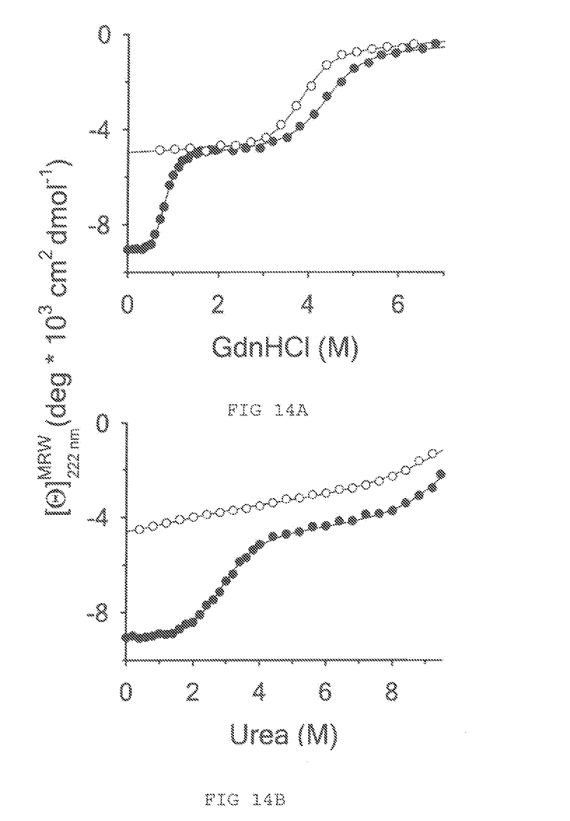

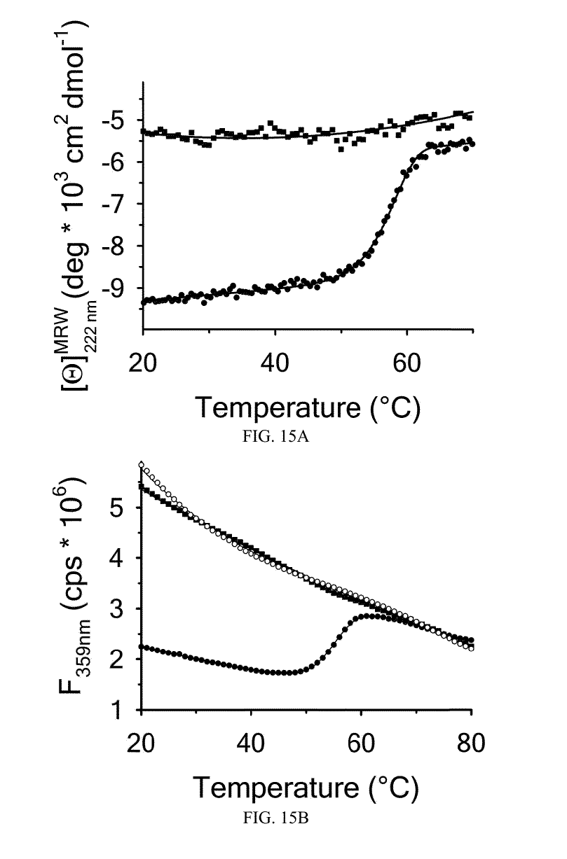

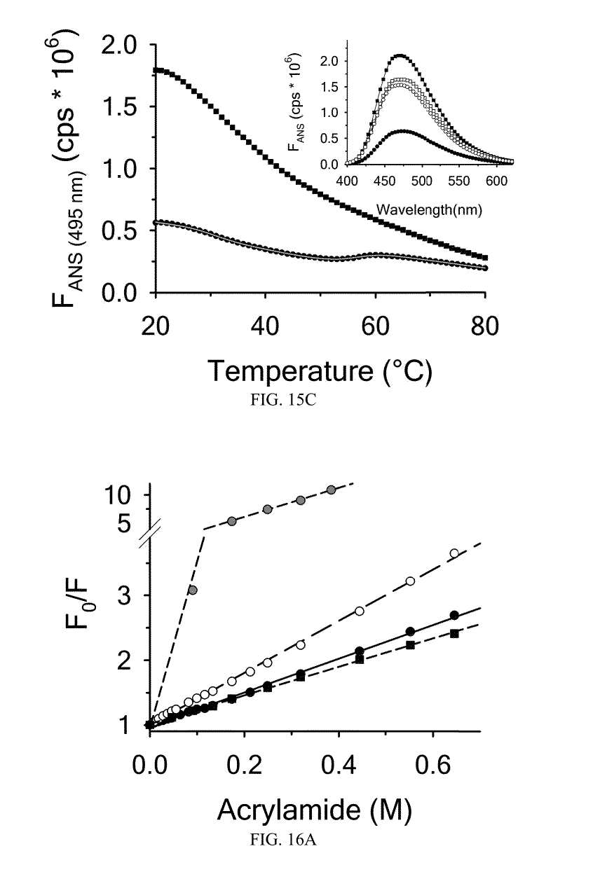

The unfolding of disulfide-intact A1 and RCAM A1 was also monitored via CD at 222 nm, intrinsic protein fluorescence at 280 nm/359 nm and ANS fluorescence at 350 nm/495 nm as a function of temperature. As thermal unfolding of the A1 domain is not reversible, the thermal transitions were analyzed using a two state irreversible model described in detail previously and all three spectroscopic methods resulted in comparable unfolding enthalpies and transition temperatures for A1, Table 3 (Tischer:2013) FIGS. 15A to 15C illustrate that all three methods resulted in a cooperative unfolding transition for disulfide-intact A1 that was absent for RCAM A1.

TABLE 3
Irreversible Thermal Denaturation Parameters.

| Method | T* (° C.) | ΔH* (kJ/mol) |
|---|---|---|
| Circular Dichroism | 65 | 272 |
| Intrinsic Fluorescence | 63 | 254 |
| ANS Fluorescence | 64.4 | 267 |

The circular dichroic ellipticity of disulfide-intact A1 changed from ˜−9.4 to ˜−5.5 upon thermal denaturation, FIG. 15A. Ellipticity of the thermally denatured state was comparable to that of RCAM A1 in presence of 1 M urea, ˜−5.0. The ellipticity of RCAM A1 was constant over the full range of temperature. RCAM A1 has a slight tendency for aggregation followed by precipitation even in presence of 1 M urea at high temperature. A thermal scan of RCAM A1 in absence of urea was significantly affected by protein aggregation beginning at ˜40° Celsius. The high tyrosine and tryptophan fluorescence intensity of RCAM A1 relative to disulfide-intact A1 indicated that most of the fluorophors were solvent exposed even at 20° Celsius, FIG. 15B. Thermal unfolding of RCAM A1 was comparable to disulfide-intact A1 in presence of 9 M urea.

Thermal denaturation in the presence of ANS was comparable to CD and intrinsic fluorescence, FIG. 15C. Spectra of the ANS fluorescence measured before thermal denaturation resulted a low initial ANS fluorescence for the disulfide-intact A1 domain indicating a relatively low exposure of hydrophobic surfaces. In contrast, the ANS spectrum of RCAM A1 resulted in significantly enhanced fluorescence intensity due to exposed hydrophobic surfaces. After each thermalscan, the protein samples were equilibrated at 20° Celsius and spectra were measured. The ANS fluorescence increased for disulfide-intact A1 and decreased slightly for RCAM A1 probably as a result of high temperature protein aggregation during the thermalscan.

Acrylamide Quenching of Tryptophan Fluorescence.

The spectroscopic characterization of A1 and of RCAM A1 showed that RCAM A1 corresponds well to the intermediate state of A1, which still contains a high amount of secondary structure, but lacks well-ordered tertiary structure or a hydrophobic core. This was also confirmed via acrylamide induced tryotophan fluorescence quenching performed with an excitation wavelength of 295 nm. The extent of the acrylamide quenching and hence the accessibility of the tryptophan residue was determined via the Stern Volmer equation and the resulting Stern-Volmer-constant.

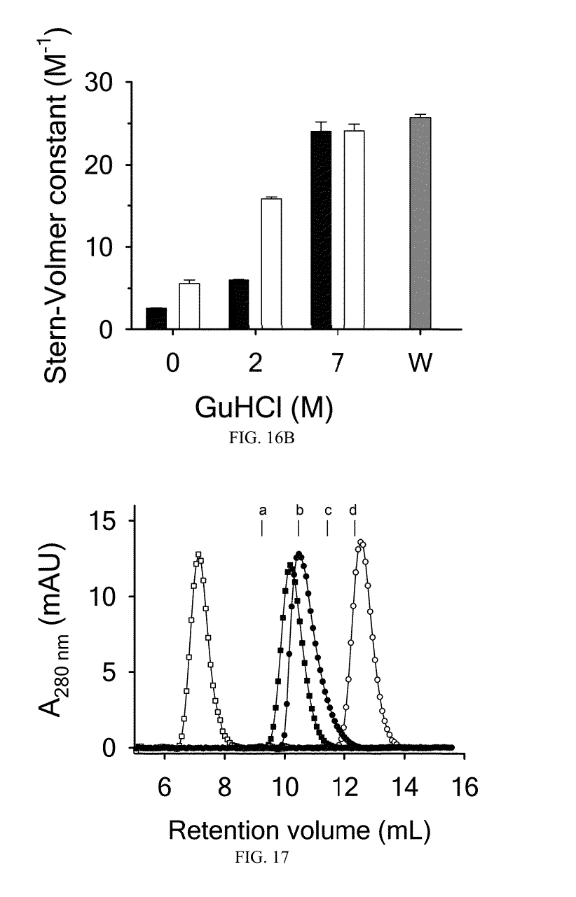

Initially a quenching determination was performed with disulfide-intact A1 at 20° Celsius and at 1° Celsius in order to distinguish between static and collisional quenching. Since the frequency of collisions between two molecules decreases at lower temperatures, the efficiency of a collisional quencher is reduced at lower temperatures and this results in a decrease Stern-Volmer constant. Clear evidence for collisional quenching could be obtained for the A1-domain since the Stern-Volmer constant decreased from 2.6±0.03 to 2.2±0.03 M−1, FIG. 16A, ruling out a static quenching mechanism.

In further quenching studies the accessibility of the tryptophan residue was determined for disulfide-intact A1 and RCAM A1 in 0, 2 and 7 M GdnHCl (FIG. 16B. In the presence of 2M GdnHCl, the Stern-Volmer constants for both protein variants increased relative to the absence of GdnHCl. In 7 M GdnHCl, these quenching constants for both disulfide-intact A1 and RCAM A1 were comparable to the quenching of NATA in water. The Stern Volmer constant of RCAM A1 in buffer was 5.6±0.4 M−1, comparable to disulfide-intact A1 in 2 M GdnHCl (6.0±0.1 M−1). Increasing GdnHCl concentrations reduced the efficiency of acrylamide quenching which is likely due to favorable interactions between GdnHCl and the tryptophan side chain which compete with collisional interactions between tryptophan and acrylamide (Nozaki:1970).

Analytical Size Exclusion Chromatography.

Size exclusion chromatography was performed to determine the apparent hydrodynamic radius of RCAM A1 relative to disulfide intact A1. In agreement with our previously reported results, the elution volume in buffer for RCAM A1 was higher (12.56 mL) than for disulfide-intact A1 (10.48 mL)(Auton:2010). While the elution volume for WT A1 corresponded well to a protein of ˜28 kDa, RCAM A1 eluted at a volume characteristic for a protein of ˜12 kDa. This atypical elution behavior indicates a partial unfolded protein that behaves like a significantly smaller protein when partitioning between the mobile and solid phases of the sizing column. However, the addition of 0.25M GdnHCl, resulted in a more expanded form of RCAM A1 that eluted at 7.13 mL relative to 10.19 mL for disulfide intact A1 in 0.25M GdnHCl. Elution of both proteins in the presence of 2M GdnHCl resulted in highly expanded conformations that approached the void volume of the column, 6.8 mL. Unfortunately, the low solubility of RCAM A1 precluded any direct measure of the size and shape of the protein due to limited detection by more direct measures such as analytical ultracentrifugation, dynamic light scattering or small angle X-ray scattering.

Reduction and carboxyamidation of the disulfide bond preferentially stabilizes a conformation that is spectroscopically similar to the urea- and GdnHCl-induced intermediate conformation of disulfide-intact A1 domain in the far UV CD. This unrestrained conformation exhibited an enhanced apparent binding affinity for GPIbα on fixed platelets that enabled a quantitative thermodynamic description of how von Willebrand disease mutations alter the coupling between shear force dependent dissociation from GPIbα and the conformational stability of A1 (Auton:2010). Here the inventor shows that RCAM A1 arrests platelets resulting in net zero translocation velocities. This is in stark contrast to disulfide-intact A1 where platelets are observed to stick and roll on the surface captured domain. Pause times determined for platelets translocating on disulfide-intact A1 represent the average length of time a platelet is immobile. This time has a complex dependence on the shear rate, which is seen to decrease at low shear, increase to a maximum at intermediate shear and then decrease again at higher shear rates. In single molecule studies designed to measure the lifetime of a bond between two proteins as a function of applied force, this behavior is referred to as a catch-slip interaction where the bond initially becomes weaker at low force, strengthens at intermediate forces, and weakens again at higher forces (Thomas:2008, Thomas:2008).

In these studies, the mean pause times for disulfide-intact A1 are longer than previously reported flow chamber measurements (Coburn:2011) and longer than single bond lifetime measurements by AFM (Yago:2008) indicating that multiple bonds between surface captured A1 and platelet GPIbα may be occurring. Multiple bonds might also explain the complex shear dependence of the apparent rate constants obtained from the pause time survival fraction (FIGS. 11B and 11C). Although others have reported rolling velocities of platelets or GPIbα coated microspheres on immobilized A1 at ≦100 s−1 (Yago:2008), these studies show consistent results with the low shear regime is that the interaction is so weak that platelets dissociate from the surface captured domain at less than 300 s−1. The inventor attributes these differences to the method of immobilization in which our protein is captured by the 6×His-tag rather than by nonspecific interactions between the A1 domain and glass or plastic surfaces. This method of surface capture by specific chelation chemistry preserves the structure of the domain and allows one to distinguish the effects of different A1 domain conformations on platelet adhesion.

Spectroscopic studies designed to interrogate the globular structure and stability of RCAM A1 confirm that breaking the disulfide linkage abolishes the native state structure. This loss of native structure is evident in 1) the increased wavelength of maximal fluorescence emission; 2) loss of circular dichroism in both the near and far UV; 3) the absence of an N<->I far UV CD transition in the chemical denaturation; 4) the absence of a thermal unfolding transition monitored by CD, FL and ANS; 5) increased intrinsic protein tyrosine and tryptophan fluorescence; 6) increased ANS binding and fluorescence; and 7) an increased Stern-Volmer constant due to collisional acrylamide quenching of solvent exposed tryptophan. Taken together, all of these spectroscopic observables demonstrate that RCAM A1 adopts a molten globule state that is devoid of tertiary structure contacts with exposed fluorophores and hydrophobic residues and only residual secondary structure content remains. Furthermore, ANS is commonly used as a molten globule indicator dye because it has a stronger affinity for proteins with prominent secondary structure but loosely packed tertiary structure elements compared with its affinity for native and random coil proteins (Baldwin:2013, Semisotnov:1991). It is commonly observed that molten globule proteins exhibit larger hydrodynamic radii than their native state conformations (Uversky:2002) While ANS is indicator of a molten globule state due to exposed hydrophobic surfaces, the SEC data show that RCAM A1 has a greater elution time than disulfide-intact A1 suggesting that the macromolecular dimensions of RCAM A1 are smaller than native A1. Near UV CD demonstrates that the tertiary structure of RCAM A1 is largely diminished if not completely absent. The inventor attributes the atypical SEC elution of RCAM A1 relative to native A1 to be due to the presence of significant amounts of supersecondary structure that could alter both the size as well as the shape of the protein in buffer. Small amounts of GdnHCl disrupt these structures due to stacking interactions that cause a greater expansion of RCAM A1 relative to native A1 (FIG. 17) (Lim:2009).

The co-crystal structure of the disulfide-intact A1 domain in complex with GPIbα shows two hydrogen-bonded contact interactions between 1) the A1-β3 strand and the β-hairpin switch of GPIbα and 2) α1β2 and β3α2 loops of A1 and the N-terminal β-hairpin of GPIbα flanking a highly solvated electrostatic surface area (Huizinga:2002). Aside from the electrostatics, these contact interactions are weak in the presence of shear due to the very few hydrogen bonds that make up the interaction. Furthermore, the GPIbα β-hairpin switch must transition from a coil conformation in the unbound state in order to accommodate binding (Huizinga:2002, Uff:2002).

The finding that RCAM A1 binds platelets tighter than disulfide-intact A1 is intriguing and counter intuitive. How can loss of structure result in gain of function? The interpretation provided herein, but in no way a limitation of the present invention, is that relaxing the domain structure by reducing the disulfide bond allows the protein to reorganize and form the secondary and supersecondary structural elements that are naturally expressed in the amino acid sequence. These secondary elements are stable in and of themselves and predetermined with a high affinity for the platelet GPIb receptor. Within this context, folding of the A1 domain, by linking the N- and C-terminal cysteines is inhibitory for the A1 GPIbα binding interaction, but not completely. Those secondary elements required for binding remain intact in the disulfide-linked domain and the process of folding and disulfide-bonding induces strain on these elements that reduces GPIb. This results in transient platelet interactions and the lack of stable adhesion to surface-captured disulfide-intact A1, observed as stick and roll translocations in the flow chamber. Releasing these secondary structure elements from the confines of the tertiary fold enables high affinity and high strength bonds with platelets. To support this idea, a small cyclic helical peptide was recently co-crystallized with GPIbα that inhibits the vWF-GPIbα interaction through binding between the concave surface of the leucine-rich repeats and the extended regulatory β-hairpin loop (McEwan:2009). Although it is not yet clear which secondary structure elements of the molten globule conformation interact with GPIbα, it is plausible that helical regions of A1 could interact in a similar manner as the inhibitor complex. It is therefore probable that the structural basis for the high affinity interaction between GPIbα and the molten globule conformation of A1 could be completely different than the native co-crystal complex portrays.

It is also conceivable that RCAM A1 interacts with other domains of the GPIb-IX-V complex or other receptors on platelet membranes. The vWF A1 domain is already known to bind heparin and collagen in addition to GPIbα and the equilibrium between the native conformation and a dynamic molten globule increases the probability of additional multivalency with respect to new protein ligands. Indeed, specific binding interactions can occur in molten globules. For example, small ligands bind to periplasmic binding proteins in the acid-induced molten globule state without conversion to the native state (Prajapati:2007). Disulfide intact vWF A1 domain also adopts a molten globule conformation at low pH like many other proteins including ribonuclease A, human α-lactalbumin and retinol binding protein (Miyata:1996, Vassilenko:2002) Similar to the A1 domain, α-lactalbumin forms a compact molten globule in the absence of disulfide bonds (Redfield:1999) Furthermore, the secondary hemostatic chemokine platelet factor 4 (PF4) that is released from activated platelets to promote coagulation though interactions with heparin, also adopts a molten globule conformation in the absence of disulfide bonds (Mayo:1992). In contrast to RCAM A1, which gains affinity for platelets in the molten globule conformation, PF4 looses affinity for heparin upon reduction of the disulfide bonds.

The high affinity molten globule state adopted by the A1 domain in the disulfide reduced form presents a considerable challenge for primary hemostasis in patients that harbor mutations that break the disulfide bond. Clearly there are both intracellular and extracellular mechanisms involved that reduce the probability of systemic thrombocytopenia. In the case reported for the patient with C1272F, plasma vWF multimer gel analysis showed an absence of high and intermediate molecular weight multimers (Woods:2012). However, only high molecular weight multimers were absent in a patient with a C1272S substitution, indicating that vWF can be secreted into the blood as low molecular weight forms (Penas:2004) Multimer studies on recombinant vWF containing C1272R and C1272G show an absence of high molecular weight multimers and an absence of both high and intermediate molecular weight species in C1458G vWF (Siguret:1996). Therefore, cross-linking the disulfide to induce the tertiary folding of A1 is required for efficient multimerization. A free cysteine in the A1 domain, due to the mutation of its partner, has the potential to pair with other cysteines in vWF resulting in incorrect linkages that could alter the quaternary structure required for multimerization. Similarly, other cysteine mutations in the D3 and B domains also result in defective multimerization that induces intracellular retention of vWF causing severe von Willebrand disease (Tjernberg:2006, Hommais:2006).

Since the single A1 domain with the reduced disulfide is sufficient to arrest translocation of platelets under shear flow, small molecular weight multimers containing an A1 domain cysteine mutation that are secreted could still pose a potential risk for microvascular thrombosis. At the level of the single A1 domain, loss of the disulfide bond is comparable to a severe type 2B gain of function phenotype. In severe cases, Type 2B VWD is also associated with reduced high molecular weight multimers and thrombocytopenia. Because of this functional similarity, ADAMTS13 could have a role in the clearance of affected vWF multimers. Increased levels of vWF pro-peptide relative to mature vWF in 2B vWD indicate an accelerated clearance of mutant vWF from the blood (Casari:2013, Casonato:2010). It has been recently demonstrated in mice that type 2B mutations are associated with an increase in satellite bands in low molecular weight multimers and a decrease in the circulatory half-life of vWF and vWF-platelet complexes (Casari:2013, Rayes:2010). Recombinant type 2B vWF proteolysis by recombinant ADAMTS13 is also enhanced relative to normal vWF (Rayes:2007) However, evidence to the contrary demonstrates that ADAMTS13 does not proteolyze the A2 domain in recombinant C1272S vWF either in the absence or presence of urea (Hassenpflug:2006). Furthermore, the kinetics of normal murine vWF clearance from plasma in ADAMTS13 deficient mice was not significantly different from mice expressing ADAMTS13 (Casari:2013, Lenting:2007, Badirou:2010). Similar to other cysteine mutations (Schooten:2005), it is plausible that A1 domain cysteine mutations could also result in increased clearance of vWF independent of ADAMTS13 due to a disrupted structural integrity of the multimers. Heterozygosity could present some measure of protection since not all monomeric units of multimeric vWF would lack the disulfide bond in the A1 domain. However, it is questionable whether low molecular weight multimers present in patient plasma contain molten globule A1 domains with a cysteine mutation or if they only contain normal disulfide linked A1 domain due to the compensation of the unaffected allele. This would be a reasonable possibility because part of the function of the endoplasmic reticulum (ER) is the retention of misfolded proteins for degradation (Lippincott Schwartz:2000), although not all misfolded proteins are retained in the ER (Kincaid:2007).

In conclusion, loss of the A1 domain disulfide bond due to genetic mutation induces a type 2A VWD phenotype because of the loss of mid- to high molecular weight multimeric forms of VWF. The inventor has assessed the functional consequence of a disulfide loss in the single A1 domain and found that it has properties of an extreme gain of function similar to type 2B VWD. This enhanced platelet interaction results from the spontaneous conversion of A1 to a molten globule conformation that retains secondary structural elements, which enable high affinity interactions with platelet GPIb. Which secondary structural elements in the A1 domain are involved in this interaction is still under investigation. Despite a severe gain of function at the single A1 domain level, vWD patients afflicted with A1 domain cysteine mutations do not show evidence of enhanced platelet-vWF interactions characteristic of type 2B vWD. This is likely a result of a number of possible protective checkpoints occurring throughout the biosynthetic, secretory and clearance pathways of vWF that depend on the proper folding and linkage of the disulfide in the A1 domain.

Tris HCl, GdnHCl, Tween 20 and Na₂HPO₄ were obtained from Fisher Scientific; sodium acetate and EDTA from ICN Biomedicals; acrylamide from Bio-rad and urea, glycine, NaCl, iodoacetamide, dithiothreitol, N-acetyl-tryptophanamide (NATA) and 8-Anilino-1-naphthalenesulfonic acid (ANS) from Sigma-Aldrich. All chemicals were of analytical grade or higher purity.

Protein Expression, Purification, Isolation and Synthesis.

The VWF A1 domain (amino acids Q1238-P1471) was expressed and purified as described previously (Tischer:2013) The reduction of A1 in buffer containing 2 M GdnHCl with 6 mM DTT followed by the carboxyamidation with 12 mM iodoacetamide was performed as described previously (Auton:2010) A1 was stored on ice in 150 mM NaCl, 25 mM Tris HCl, pH=7.4 (TBS). RCAM A1 was stored on ice in TBS+2M GdnHCl. All functional studies were performed in TBS. For all thermodynamic and spectroscopic studies, proteins were dialyzed overnight against PGA Buffer, 10 mM sodium acetate, 10 mM Na₂HPO₄, 10 mM Glycine, 150 mM NaCl, 1 mM EDTA, pH=8 before use.

Concentration Determination.

Protein, NATA, ANS and peptide concentrations were determined photometrically on a Shimadzu UV2101PC spectrophotometer using the Edelhoch method as modified by Pace (Pace:1995) from absorption at λ=280 nm minus twice the absorption at λ=333 nm for correction of light scattering. Extinction coefficients for A1 and RCAM A1 ε=15350 L/mol/cm) were calculated from eight tyrosines and one tryptophan. The extinction coefficient for NATA is 5630 L/mol/cm and the concentration of ANS-solutions was determined at λ=350 nm using an extinction coefficient of 4950 L/mol/cm (Motono:1999).

Parallel Plate Flow Chamber Studies.

The determination and analysis of platelet pause times and instantaneous translocation velocities over surface captured A1 and RCAM A1 was performed as previously described (Tischer:2013). Here, the inventor used the Cellix Mims Evo syringe pump controlled by VenaFluxAssay (www.cellixltd.com). Vena8™ CGS biochips were custom designed with 800 μm width by 80 μm height microchannels bonded to glass slides with a Cu²⁺ chelated PEG surface serviced by Microsurfaces Inc. (www.proteinslides.com/histag.html) to which the A1 and RCAM A1 in TBS were captured by the 6×His-Tag at a total concentration of 5 μM. Citrated whole blood was obtained from the informed consent of healthy volunteer donors with approval from the Mayo Clinic Institutional Review Board and 100 μL was perfused over the surface captured A1 domains at 700 s⁻¹. The shear was increased or decreased in a stepwise manner to measure the dynamics of platelet adhesion at high and low shear. After allowing the flow chamber to equilibrate for 50 s at each shear rate, 1 min movies were recorded at 24 frames per second in phase contrast at 200× magnification with 2×2 pixel binning on a Zeiss AxioCam Mrm camera (6.45 μm/px) attached to a Zeiss Axio Observer.D1 inverted microscope. Tracking analysis was performed using MediaCybernetics ImagePro® Premier (www.mediacy.com/index.aspx?page=IP\_Premier) The distance travelled in the direction of flow was calculated from the coordinate data for each platelet as a function of time using \sqrt{(xₜ−x₀)²}, where x₀ is the coordinates of a platelet on the first frame of the movie and xₜ is the coordinate on subsequent frames of the movie. These trajectories were differentiated into instantaneous velocities using a Savitzky-Golay algorithm (Savitzky:1964) with a five data point window size and a second order polynomial implemented into a Mathematica (www.wolfram.com/mathematica) notebook written in our lab. Trajectories were retained for statistical analysis if the platelet was present for ≧1 s and travelled a total distance greater than 1 Distance travelled in pixels (px) was converted to (μm) using 6.45 μm/px*2/200=0.0645 μm/px. Pause times were determined by the amount of time (sec) a platelets velocity was 0±4 px/s≅0±0.23 μm/s, within the noise. Data are reported as mean velocities and pause times attained by individual platelets averaged over all platelets analyzed. Integral analysis of the cumulative pause time histogram gives the survival fraction, equation (24).

$$\frac{\# {PT}_{total} - {\sum }_{t = 0}^{t} \# {PT}_{t}}{\# {PT}_{total}} \qquad ( 24 )$$

where # PTₜₒₜₐₗ is the total number of pause times counted and # PTₜ is the total number of pause times that are ≦ to time t. A bi-exponential decay function was fit to the fraction survival data to obtain estimates of the dissociation rates and amplitudes at each shear rate investigated.

CD Spectroscopy.

Urea and GdnHCl induced unfolding of A1 and of RCAM A1 at 25° Celsius was monitored via CD at 222 nm on an Aviv Biomedical Model 420C circular dichroism spectrometer. A 1 mm quartz cuvette and protein concentrations of 5 or 10 μM were used. Samples were equilibrated in their urea or GdnHCl containing buffer at 25° Celsius overnight. CD-signal was averaged for 5 to 10 min using an integration time of 1 s. CD-spectra were measured at 20° Celsius using 0.1 or 1 mm quartz cells with a step width of 1 nm between 190 and 260 nm with an integration time of 20 s and a bandwidth of 1 nm. CD-spectra in the near UV range (260-360 nm) were recorded using a 5 cm quartz cell with a 120 s integration time. Transition curves and protein spectra were corrected for the CD Signal of the corresponding buffer.

CD thermalscans were recorded between 10 and 95° Celsius at 222 nm with a protein concentration of 1 μM in a 1 cm quartz cell under moderate stirring. Prior to each measurement protein samples were equilibrated at 10° Celsius for 10-15 min in order to obtain a stable baseline. The scanrate for all Thermalscans was 2° Celsius/min. The integration time for each data point was 20 s, the bandwidth 1 nm. All measured CD data were converted into mean molar ellipticities per amino acid residue.

Fluorescence Spectroscopy.

All fluorescence measurements were performed on a Horiba Jobin-Yvon Fluorolog® 3 spectrofluorometer equipped with a Wavelength Electronics Model LF1-3751 temperature controller. Protein fluorescence emission spectra of 2 μM A1 and RCAM A1 were averaged three times between 305 and 440 nm with excitation at 280 nm. The step width was 1 nm and the integration time 1 s. ANS fluorescence emission spectra were averaged three times between 400 and 620 nm with excitation at 350 nm. Prior to the measurements 1 μM A1 or RCAM A1 was incubated for one hour at 20° Celsius with 100 μM ANS. All fluorescence spectra were corrected for the signal of the corresponding buffer.

Thermalscans monitoring protein fluorescence were recorded at an emission wavelength of 359 nm after excitation at 280 nm using a protein concentration of 2 μM in a 1 cm quartz cell under moderate stirring. ANS-Thermalscans were recorded at 495 nm after Excitation at 350 nm using 1 μM protein and 100 μM ANS. Prior to any performed thermalscan samples were equilibrated for 10-15 min. The scanrate was 2° Celsius/min. At each temperature between 10 or 20° Celsius and 95° Celsius relative fluorescence intensity was collected for 4 s and averaged.

Acrylamide quenching of tryptophan was performed using an excitation wavelength of 295 nm. After the addition of a certain amount of buffered acrylamide solution the protein solution was equilibrated for ˜5 min and then a fluorescence spectrum between 320 and 400 nm was recorded. Stern-Volmer constants were determined from the fluorescence intensity at 359 nm using the Stern-Volmer equation as described by Lakowicz (Lakowicz:2006)

$$\frac{{F}^{0}}{F} = 1 + {K}_{D} [ Q ] \qquad ( 25 )$$

where K_{D} is the Stern-Volmer quenching constant and K_{D-1} is the concentration of acrylamide at which F₀/F=2 and 50% of the fluorescence intensity is quenched.

Analytical Size-Exclusion Chromatography.

Analytical size-exclusion chromatography was performed at 4° Celsius using a Superdex 75 column with a total bed volume of 24 mL on an ÄKTAFPLC-system (GE Healthcare Life Sciences). The column was equilibrated with PGA buffer containing 0M, 0.25M and 2M GdnHCl and 500 μL of WT A1 (5 μM) and RCAM A1 (5 μM) were injected followed by elution at 0.5 mL/min flow rate.

Thus, the present invention provides a statistically robust and quantitative assay for vWF-platelet function capable of distinguishing between gain and loss-of-function and discerning differences in the extent to which mutations alter function. The apparatus and methods taught herein can thus be used to identify and estimate the severity of bleeding in patients of known genotype.

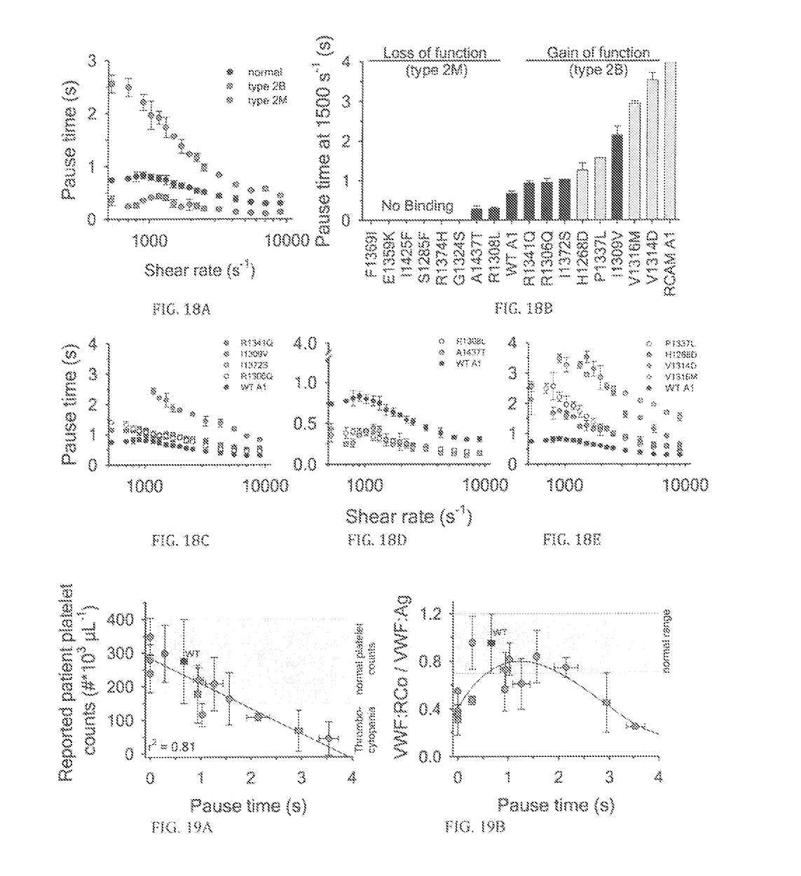

FIGS. 18A to 18E summarize the clinical correlation of pause times and varying low level binding interactions with specific VWD mutations. FIG. 18A: the Shear rate dependency of platelet pause times on representative normal (WT A1), type 2B (P1337L) and type 2M (A1437T). FIG. 18B shows the rank order of pause times at 1500 s⁻¹ shear rate from least to greatest. The type 2M variants F1369I, E1359K, I1425F, S1285F and R1374H did not bind platelets at any shear rate. Grey bars indicate “molten globule” type 2B variants of A1 domain. Shear rate dependency of platelet pause times on FIG. 18C “natively structured” type 2B A1 domain variants, FIG. 18D “natively structured” type 2M A1 domain variants, and FIG. 18E “molten globule” A1 domain variants.

FIGS. 19A and 19B show, FIG. 19A are the platelet counts reported for vWD patients with known mutations as a function of the platelet pause times obtained at 1500 s⁻¹ shear rate shown in FIG. 2. The linearity has a correlation coefficient of determination (R2=0:81). FIG. 19B shows the ratio of vWF ristocetin cofactor activity relative to vWF antigen levels reported for vWD patients. Grey areas indicate the normal range of these clinical metrics. 2B=red, 2M=blue. Data points at zero pause time represent the type 2M mutations for which there was no shear dependent binding. These data are also tabulated in Table 4.

TABLE 4
Summarizes the structure/function properties of Type 2 VWD variants of the A1
domain and their clinical manifestations in VWD patients and the pause times measured using the
present invention.

| Mutation | A1 domain | Pause Time | VWF:RCo/ | Patient Platelet | Patient VWF | |
|---|---|---|---|---|---|---|
| (VWD type)^{R} | Conformation^{a} | 1500 s⁻¹ (s)^{b} | VWF:Ag^{c} | Count (*10³/μL)^{c} | Multimers^{d} | Unique Symptoms^{e} |
| F1369I (2M) | MG | NB (2) | 0.36 ± 0.07 (4) | NR | Normal | ↓ RiPB, Normal BiPB |
| E1359K (2M) | MG | NB (2) | 0.55 (1) | NR | NR | ↓ RiPB, Normal BiPB |
| I1425F (2M) | MG | NB (2) | 0.32 ± 0.01 (4) | NR | Normal | ↓ RiPB, Normal BiPB |
| S1285F (2M) | MG | NB (2) | 0.39 ± 0.05 (3) | 348 ± 55 (3) | ↓ HMW | ↓ RiPB, ↓ BiPB |
| R1374H (2M) | MG | NB (2) | 0.36 ± 0.18 (50) | 239 ± 56 (6) | ↓ HMW w/ATS | ↓ RiPB, ↓ BiPB |
| G1324S (2M)^{f} | N | NB (3) | 0.31 ± 0.13 (4) | 278 ± 47 (3) | Normal | Absent RiPB, Normal BiPB |
| A1437T (2M) | NL | 0.28 ± 0.07 (3) | 0.47 ± 0.64 (4) | NR | NR | Mild moderate bleeding |
| H1308L (2B) | N | 0.29 ± 0.03 (5) | 0.96 ± 0.22 (20) | 298 ± 85 (11) | Normal | ↓ Collagen binding |
| WT A1 | N | 0.67 ± 0.07 (6) | 0.7-1.2 | 150-400 | Normal | Normal |
| R1341Q (2B) | NL | 0.94 ± 0.06 (5) | 0.56 ± 0.18 (27) | 178 ± 86 (26) | absent HMW | Moderate bleeding w/TCP |
| R1306Q (2B) | N | 0.95 ± 0.1 (3) | 0.73 ± 0.14 (5) | 219 ± 36 (5) | Normal | Mild TCP |
| I1372S (2B) | N | 1.03 ± 0.004 (2) | 0.82 ± 0.13 (2) | 116 ± 34 (2) | Normal | SPA |
| H1268D (2B) | MG | 1.26 ± 0.17 (4) | 0.61 ± 0.21 (5) | 208 ± 79 (8) | ↓ HMW | Intermittent mild TCP |
| P1337L (2B) | MG | 1.56 ± 0.005 (4) | 0.84 ± 0.22 (10) | 163 ± 78 (8) | ↓ HMW | Moderate TCP |
| I1309V (2B) | N | 2.14 ± 0.23 (3) | 0.75 ± 0.08 (14) | 109 ± 11 (13) | Normal | SPA & Persistent |
| | | | | | | moderate TCP |
| V1316M (2B) | MG | 2.94 ± 0.07 (4) | 0.45 ± 0.25 (47) | 68 ± 61 (31) | absent HMW | Chronic severe TCP |
| V1314D (2B) | MG | 3.53 ± 0.19 (3) | 0.25 (1) | 45 ± 50 (2) | absent HMW | Severe TCP |

^{a}N = “Native” (native structure with varying thermodynamic stability). NL = “Native-Like” (defined by a reduced secondary structure content with a urea and thermal unfolding transition), MG = “Molten Globule” (defined by lack of a urea and thermal unfolding transition).
^{b}NB = “No Binding.” Pause times are reported as the mean ± std. dev. of the number of experimental measurements made in parentheses.
^{c}Clinical data reported as the mean ± std. dev. of all reported data in the literature referenced below. The number of reported values are in parentheses. NR = “Not Reported.” VWF:RCo/VWF:Ag ratio and platelet counts are reported for a similar G1324A substitution [4]
^{d}HMW = “high molecular weight.” ATS = “abnormal triplet structure.”
^{e}RiPB = “ristoretein-induced platelet binding.” BiBP = “botrocetin-induced platelet binding.” SPA = “spontaneous platelet aggregation.”
TCP = “thrombocytopenia.” Adjectives describing TCP are quoted directly from the authors.
^{f}Some clinical data also included from the analogous mutation G1324A due to insufficient clinical data on G1324S.
^{R}Literature reporting patient clinical data.
F1369I → [5-7].
E1359K → [8, 9].
I1425F → [5, 6, 9].
S1285F → [10].
R1374H → [6, 11, 18].
G1324S → [4, 9, 19-22].
A1437T → [23].
R1308L → [16, 17, 24-26].
R1341Q → [16, 25, 27-30].
R1306Q → [28, 31, 32].
I1372S → [33 ].
H1268D → [9, 55, 34].
P1337L → [16, 17, 17, 25, 35, 36].
I1309V → [7, 16, 25, 30, 37].
V1316M → [9, 16, 25, 28, 30, 31, 36, 38-51].
V1314D → [23].

### Example 3

### Misfolding of vWF to Pathologically Disordered Conformations Impacts the Severity of von Willebrand Disease

The primary hemostatic von Willebrand factor (vWF) functions to sequester platelets from rheological blood flow and mediates their adhesion to damaged subendothelium at sites of vascular injury. The inventor surveyed the effect of 16 disease-causing mutations identified in patients diagnosed with the bleeding diathesis disorder, von Willebrand disease (vWD), on the structure and rheology of vWF A1 domain adhesiveness to the platelet GPIbα receptor. These mutations have a dynamic phenotypical range of bleeding from lack of platelet adhesion to severe thrombocytopenia. Using new rheological tools in combination with classical thermodynamic, biophysical, and spectroscopic metrics, the inventor establish herein a high propensity of the A1 domain to misfold to pathological molten globule conformations that differentially alter the strength of platelet adhesion under shear flow. Rheodynamic analysis establishes a quantitative rank order between shear-rate-dependent platelet-translocation pause times that linearly correlate with clinically reported measures of patient platelet counts and the severity of thrombocytopenia. These results demonstrate that specific secondary structure elements remaining in these pathological conformations of the A1 domain regulate GPIba binding and the strength of vWF-platelet interactions, which affects the vWD functional phenotype and the severity of thrombocytopenia.

von Willebrand disease (vWD) constitutes a very common bleeding diathesis with an estimated epidemiological prevalence of ˜1% of the human population (1). Over the course of the last few decades, clinical diagnostics have enabled the classification of this disorder into three distinct types (vWD types 1-3) of von Willebrand factor (vWF) deficiency. vWF, a multimeric glycoprotein secreted from the vascular endothelium, mediates platelet adhesion to exposed subendothelial connective tissue and contributes to the arrest of bleeding during primary hemostasis. It consists of multiple copies of A, B, C, and D domains that are arranged in the order D′-D3-A1-A2-A3-D4-B1-B2-B3-C1-C2-(cysteine knot).

Type-1 and type-3 vWD results in partial or complete quantitative deficiencies of vWF from blood plasma. The qualitative functional deficiencies of vWF associated with type-2 vWD result in one of the following: defective interactions between the A1 domain and the GPIba platelet receptor, loss of hemostatic activity due to abnormal multimer proteolysis of the A2 domain by the ADAMTS-13 plasma protease, or deficient binding of the D domains to FVIII.

Type-1 vWD accounts for ˜65-80% of cases; type-2 vWD, ˜20-35%; and type-3 is extremely rare (2). Of 399 unique mutations identified in the vWF gene associated with vWD, 160 are associated with type-2 vWD and 54 of these directly affect binding between A1 and GPIba (3) in the vWD subtypes 2B and 2M. These subtypes categorically represent either an increased (2B) or reduced/deficient (2M) vWF-dependent platelet adhesion. Missense mutations in these vWD subtypes have a dynamic range of clinical manifestations from a paucity of vWF-platelet interactions to severe thrombocytopenia resulting in bleeding at both extremes of vWF dysfunction.

From the time of early observations of impaired platelet adhesion to the subendothelium in vWD (4) and following the isolation and characterization of the vWF gene, protein, and domain structure (5-7), the general perception has been that mutations affecting the adhesion of platelets to vWF alter the conformation of the A1 domain. This idea has been prevalent even in the early literature describing the patients afflicted by the specific missense amino-acid substitutions causing the disease. Type 2B mutations were presumed to alter the conformation of vWF multimers and increase exposure of the GPIb binding site (8) such that vWF could mimic the effect of subendothelial matrix or collagen binding, causing pathologic spontaneous platelet aggregation (SPA) (9).

Conversely, a complete loss-of-function, type 2M mutation was thought to inhibit the normal allosteric regulation of GPIb binding (10). The concept of allosterism took precedence as more mutations were identified and the regulation of vWF interaction with platelet GPIb was thought to involve an equilibrium between binding-competent and incompetent states. More recently, it has been suggested that type 2M mutations could destabilize the global fold of the A1 domain resulting in loss of function (11-13). The first evidence of distinctly different GPIb binding conformations in the recombinant A1 domain was observed when reduction and alkylation of the disulfide bond enhanced GPIb binding affinity and reversed the shear flow dependency of platelet adhesion (14,15).

From a thermodynamic perspective the inventor directly observed a stable, partially unfolded intermediate conformation of the A1 domain in the denaturant unfolding pathway (16). To model this intermediate, the inventor reduced-and-carboxyamidated (RCAM) the A1 domain and found increased affinity for platelet GPIb. Combined with unfolding studies of A1 harboring a few mutations, the inventor developed a thermodynamic cycle that described the A1-GPIb interaction in terms of an equilibrium between low- and high-affinity conformations. The model was consistent with previous concepts of allosterism while bringing protein folding to the forefront of the issue, but it did not account for situations in which the A1 domain could misfold to inactive conformations (17,18). The inventor demonstrated that RCAM A1 exists in a molten globule state, a conformation lacking global tertiary structure while retaining secondary and potential supersecondary structure elements. These structural elements in RCAM A1 are sufficient to maintain high affinity for GPIb such that platelets remain firmly attached under shear flow (19)(shown hereinabove).

In this example, the predisposition of the platelet GPIba binding A1 domain of vWF is shown to be toward a partially disordered molten globule state involving mutation-induced structural abnormalities that specifically alter the shear-dependent function of vWF. The inventor recombinantly engineered 16 of the most commonly reported type 2B and 2M mutations into the A1 domain of vWF and compared their effect on shear-dependent platelet adhesion and the conformational and thermodynamic properties of A1. All proteins excepting five type 2M variants of the A1 domain supported shear-flow-dependent platelet adhesion to surface-captured A1 domain variants in a parallel plate flow-chamber. The strength of platelet adhesion by taking the statistical average of pause-time distributions was analyzed, which is a measure of the average time platelets remain immobile under shear flow. These pause times are significantly correlated to reported vWD patient platelet counts (R²±0.88) and the severity of thrombocytopenia indicating the structural and functional properties of the isolated A1 domain variants are representative of plasma vWF platelet interactions.

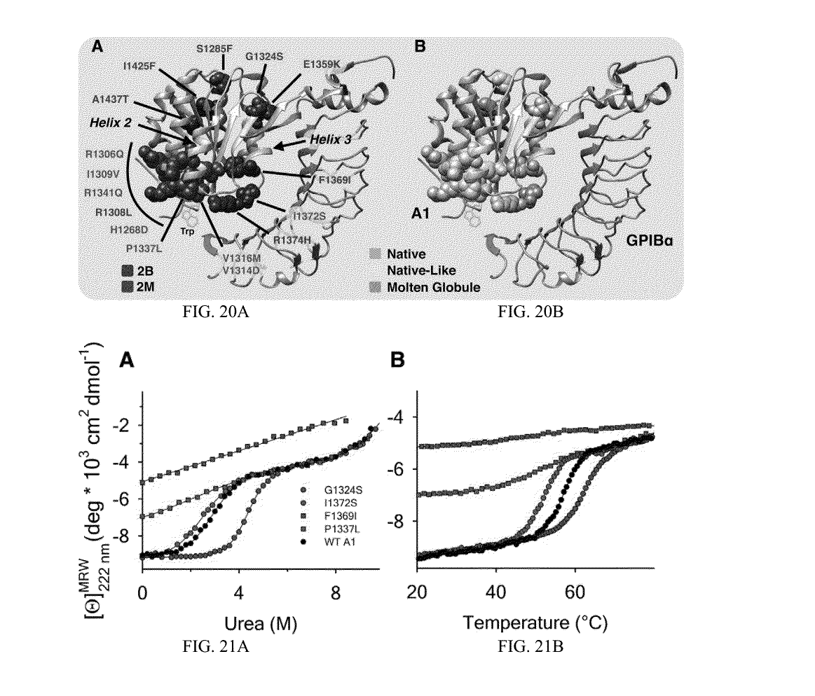

Using established conformational metrics (19), only six of the 18 variants (including WT and RCAM A1) are natively structured, and 10 of the remaining 12 variants are molten globules. One type 2M mutation reduced platelet adhesion relative to WT, five type 2M mutations resulted in molten globules with a complete loss of function, and four type 2B mutations induced molten globule states that differentially enhance platelet pause times under rheological shear flow. The structural locations of these mutations in the A1 domain (FIGS. 20A and 20B) show that allosteric effects in addition to localized effects of mutations on the a2-helix loop a3helix may regulate the adhesive strength of the A1-GPIba tether bond. These results show that the A1 domain misfolding to disordered conformations and indicate that the pathobiological function of the vWF A1 domain depends on secondary structure elements remaining in these disordered states.

Mutagenesis, Expression, Purification and Quantification of the vWF A1 Variants.

Primers for the mutation of the A1 domain in the pQE-9 plasmid vector were designed using Primer X and ordered from either Invitrogen or IDT DNA. Using these primers, mutations were generated using the Agilent Technologies Site-Directed Mutagenesis Kit. Plasmids were transformed into XL1 Blue bacteria cells. Bacteria were plated on LB agar containing 100 μg/mL ampicillin. Selected colonies were inoculated onto a new plate and screened for the correct mutation by PCR using primers from IDT generated from sequences on the pQE-9 vector or the A1 domain and run on 1% agarose gel. Plasmid bands were extracted using QIAGEN® QIAquick Gel Extraction Kit and sequenced in the Mayo Clinic sequencing lab. Colonies were also screened by growing in LB broth with 100 μg/mL ampicillin. The resulting plasmids were purified using the QIAGEN® QIAprep® Spin Miniprep Kit, digested with HindIII and BamHI restriction enzymes from New England Biolabs, and run on 1% agarose gel. Colonies with the correct mutation were grown in 250 mL LB broth with 100 μg/mL ampicillin. Plasmids were purified using the QIAGEN® EndoFree Plasmid Maxi Kit and reconstituted in TE buffer. The sequences of the plasmids were further verified by PCR and sequencing.

vWF A1 domain variants (amino acids Q₁₂₃₈-P₁₄₇₁) were expressed in E. coli, isolated and purified in the same manner as previously described for the WT A1 domain [1]. Proteins were stored at 0 C in 150 mM NaCl, 0.05% Tween 20 and 25 mM Tris HCl, pH 7.4 and dialyzed overnight against 10 mM sodium acetate, 10 mM Na₂HPO₄, 10 mM Glycine, 150 mM NaCl, 1 mM EDTA, pH 8 before use in spectroscopic experiments. Proteins used for the flow chamber experiments were dialyzed overnight exhaustively against 150 mM NaCl, 25 mM Tris HCl, pH 7.4. Protein concentrations were determined spectrophotometrically on a Shimadzu UV2101PC spectrophotometer as previously described [1, 2].

Biophysical, Structural, and Thermodynamic Metrics.

Circular dichroism measurements were performed on a Model No. 420C circular-dichroism spectrometer (Aviv Biomedical, Inc., Lakewood Township, N.J.). All fluorescence (FL) measurements were performed on a Fluorolog3 spectrofluorometer (HORIBA Jobin-Yvon, Edison, N.J.) equipped with a Model No. LF1-3751 temperature controller (Wavelength Electronics Inc., Bozeman, Mont.). Near- and far-ultraviolet (UV) circular dichroism (CD), intrinsic protein FL, and ANS (8-anilino naphthalene sulfonic acid) CD spectra were determined as described in Tischer et al. (19). Urea denaturation (monitored by far-UV CD) and thermal denaturation (monitored by far-UV CD, intrinsic protein FL, and ANS FL) were performed and analyzed as previously described (19,20). Acrylamide quenching of intrinsic tryptophan fluorescence was performed and analyzed as described in Tischer et al. (19). All spectra were collected at 20° C.

Parallel Plate Flow-Chamber Studies.

The rheological interaction of platelets with A1 domain variants harboring type 2M and type 2B mutations was studied using recently developed flow-chamber methods (19). The A1 domains (5 mM) were immobilized on Vena8™ biochips (Cellix, Dublin, Ireland) coated with a Cu²⁺-chelated poly-ethylene-glycol surface serviced by MicroSurfaces, Inc. (Englewood, N.J.) via the 6×His-Tag, enabling the preservation of the structural properties of the domain in solution and its concomitant effects on platelet adhesion under rheological shear flow. One-hundred microliters of citrated whole blood, obtained from the informed consent of normal healthy volunteer donors with approval from the Mayo Clinic Institutional Review Board (Rochester, Minn.), was perfused at a shear rate of 800 s⁻¹ followed by Tris-buffered saline. Platelets translocating on the surface-captured domains were filmed at 24 frames/s for 1 min at logarithmic intervals of the shear rate. Coordinates obtained from tracking analysis of the resulting movies were processed using the inventor recently developed statistical analytics for the quantification of cellular adhesion dynamics across immobilized receptors under rheological shear flow (supra). Biochips lacking the surface-captured A1 domain were completely inert to platelets.

Thermodynamic and Conformational Characterization of Types 2M and 2B vWF A1 Domain variants.

The effects of mutations on the thermodynamic stability of A1 (18) was determined and a framework that incorporated partial unfolding as a means for enhancing the binding affinity of A1 for GPIba was developed (17). Initially, urea-temperature phase diagram method were used for assessing the reversible thermodynamics of unfolding at various temperatures (18,21), and more recently thermal denaturations were used to assess kinetic irreversibility (20,22). The inventor employed these methods using a variety of spectroscopic observables that assess the conformation of these A1 domain variants as a function of temperature and urea.

The observables included the following: (1) Far-UV CD to assess secondary structure content; (2) Near-UV CD to assess tertiary structure content; (3) Intrinsic protein FL to assess solvent exposure of tyrosine and tryptophan residues; (4) Acrylamide quenching of tryptophan FL to assess local solvent exposure of the single tryptophan residue; and (5) Binding of the molten globule indicator-dye, ANS, to assess exposure of hydrophobic surface area.

In total, these studies have provided metrics that are able to classify the conformation of A1 into three types, based on biophysical spectroscopy, as follows:

Natively Structured.

In this class, the A1 domain is in its global native fold, but mutations induce local conformations in the native state ensemble that alter its thermodynamic stability against partial unfolding toward an intermediate conformation. The type 2B mutations R1306Q and I1309V and the type 2M mutation G1324S have previously been characterized in this conformational class (18). In addition, R1308L and I1372S also belong to this class.

Nativelike.

In this class, the A1 domain has reduced secondary structure content, but retains some of the tertiary structure required for a cooperative urea- or temperature-induced protein unfolding transition. The type 2M mutation A1437T and the type 2B mutation R1341Q belong to this class.

Molten Globule.

In this class, the A1 domain has a significantly reduced tertiary structure content as defined by the absence of a cooperative urea- or temperature-induced protein unfolding transition and retains only secondary and/or supersecondary structure. The type 2M mutations F1369I, E1359K, I1425F, S1285F, and R1374H, and the type 2B mutations H1268D, P1337L, V1316M, and V1314D, belong to this class.

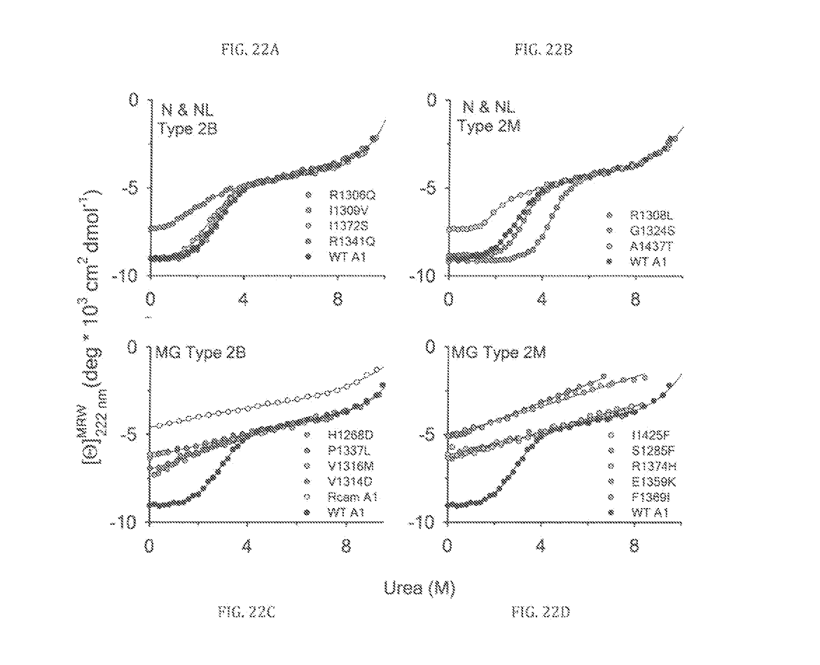

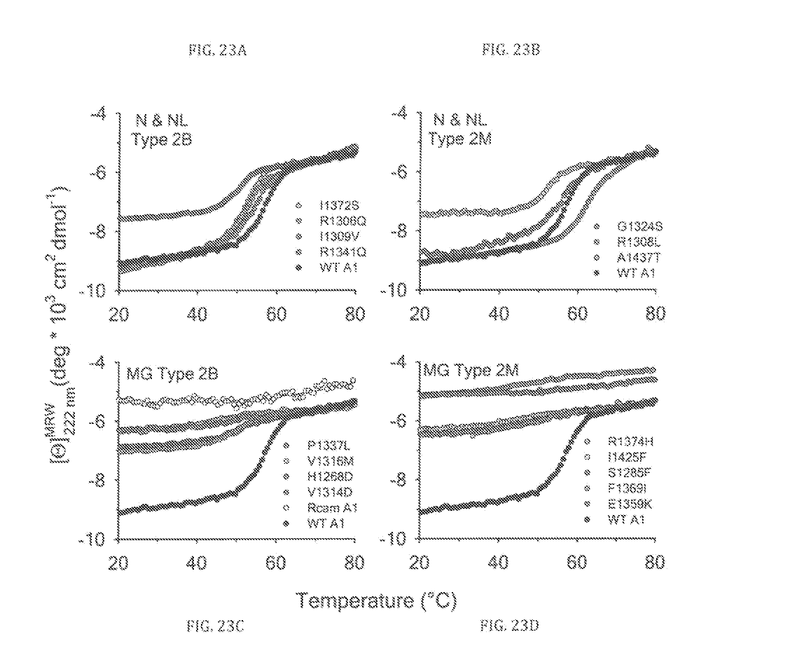

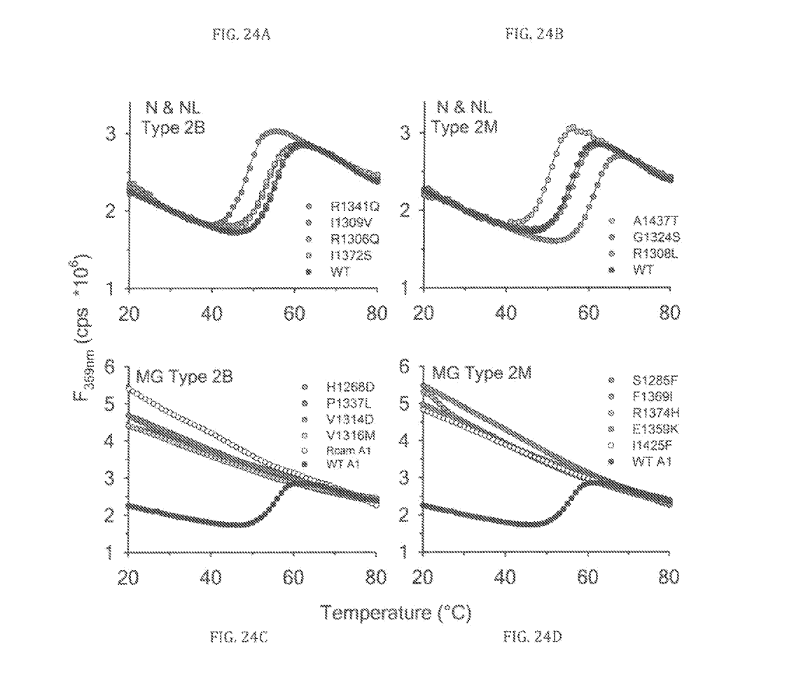

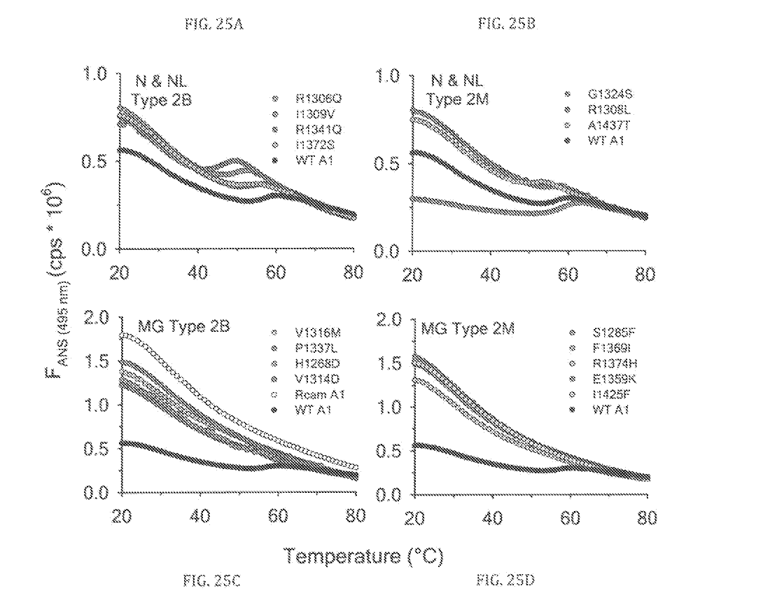

FIGS. 21A and 21B and FIG. 22, FIG. 23, FIG. 24, and FIG. 25 in the Supporting Material illustrate urea and thermal denaturations of type 2M and type 2B mutations from each conformational class monitored by Far-UV CD, intrinsic protein FL, and ANS FL for all A1-domain variants studied.

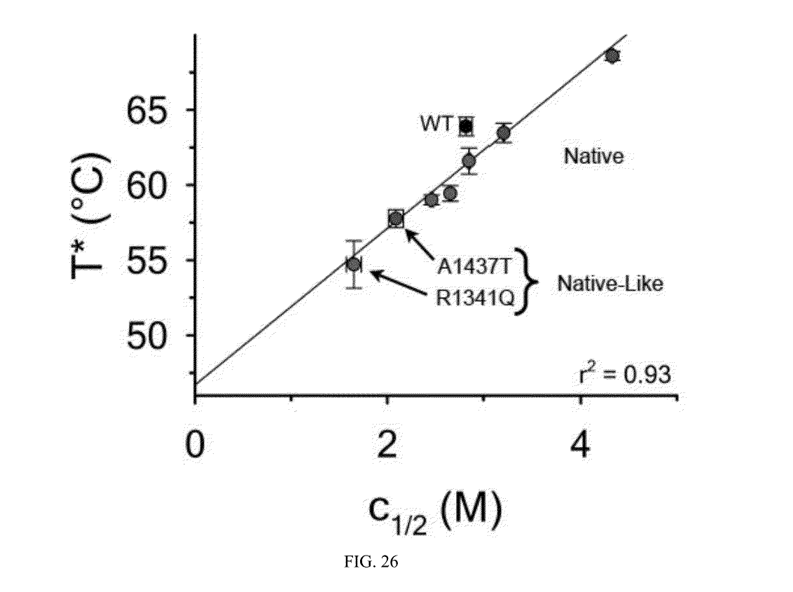

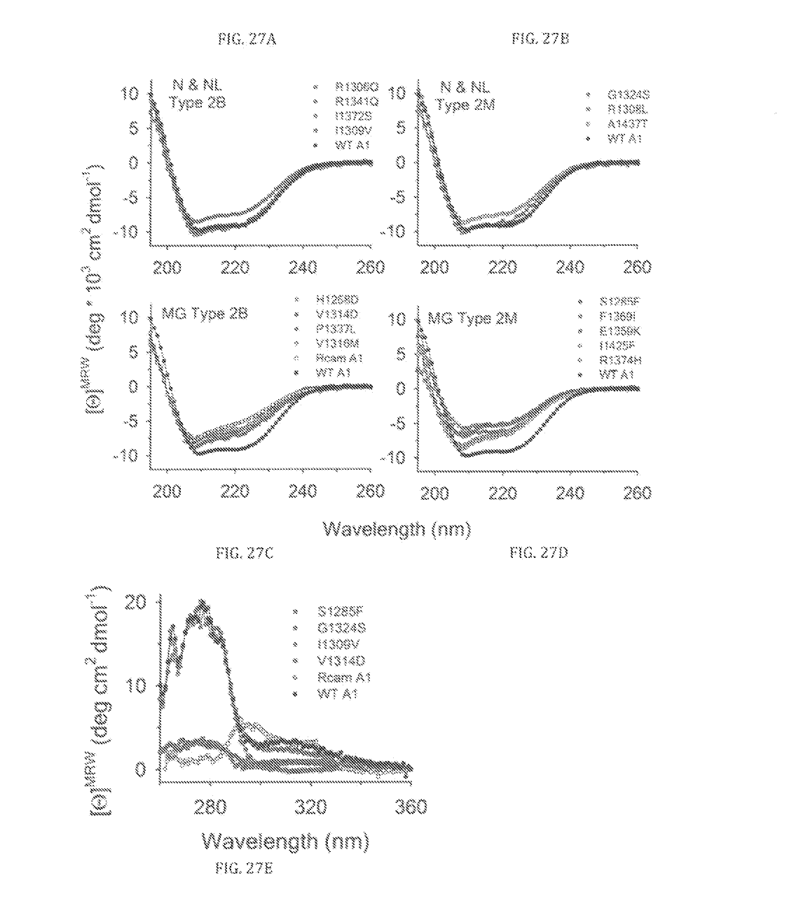

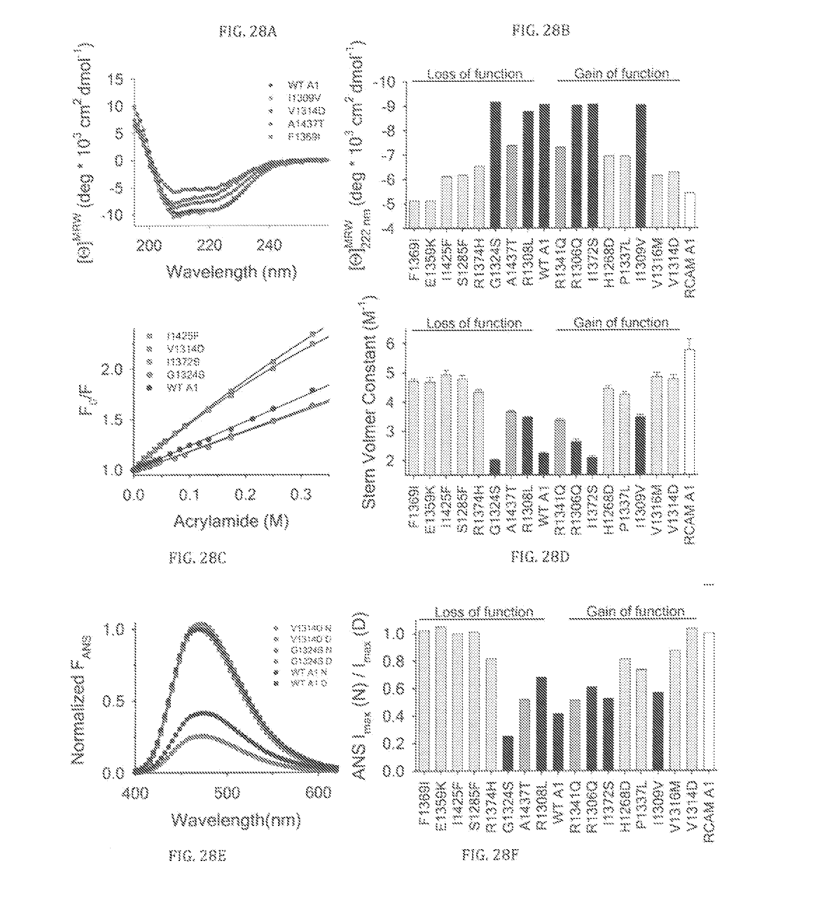

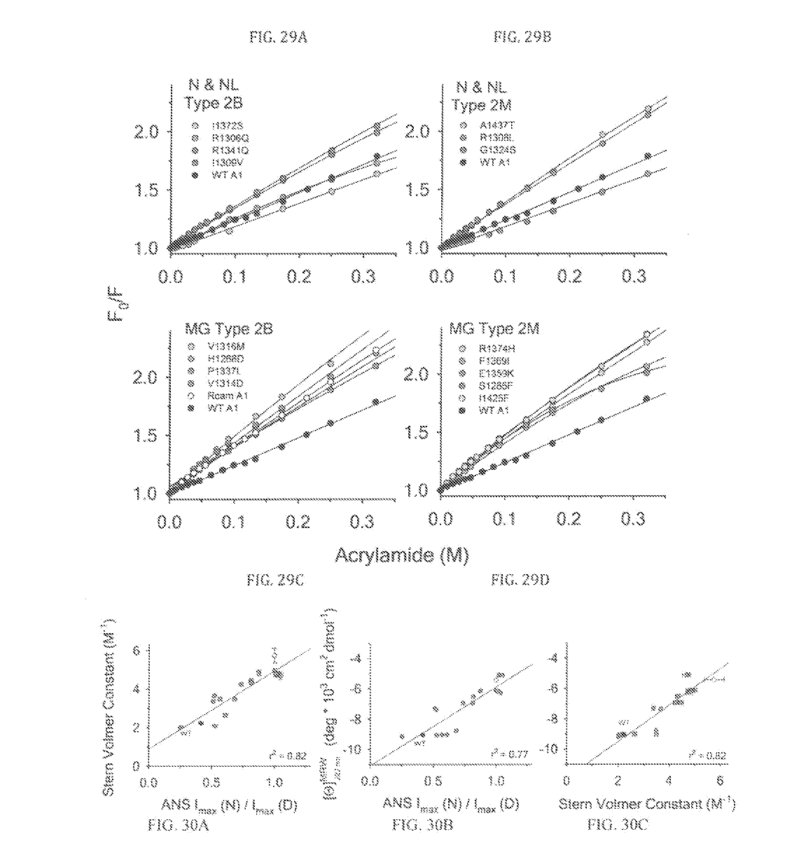

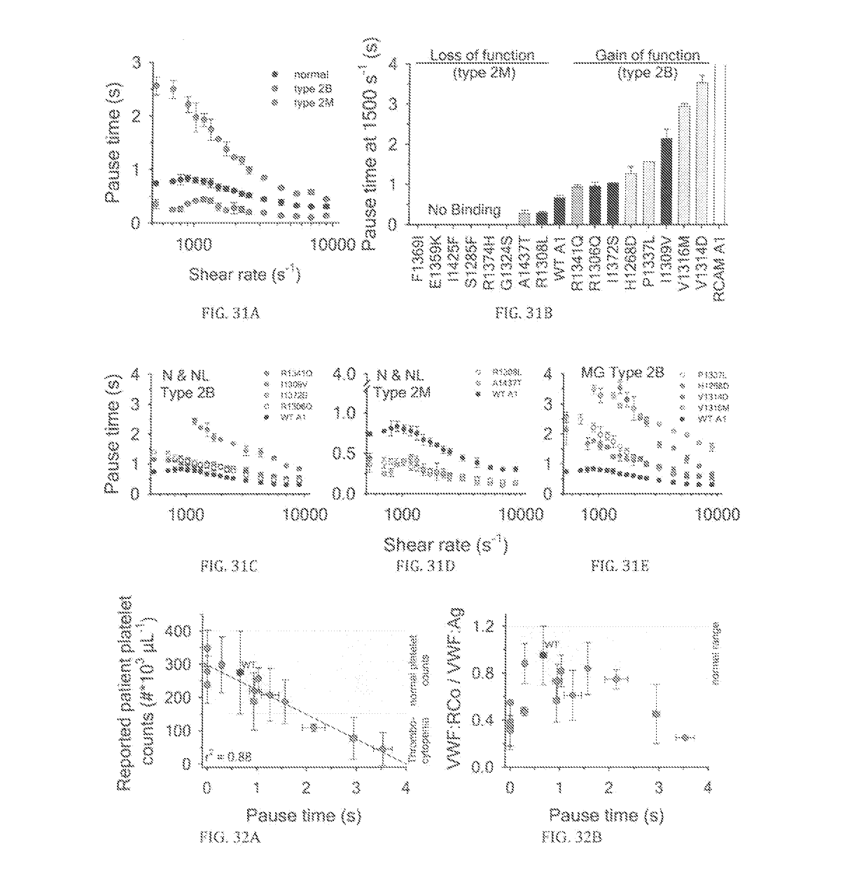

Thermodynamic analysis (see Table 5) of the native variants generally demonstrates that the stability is decreased relative to WT A1 for type 2B gain-of-function variants and the stability is increased for loss of function variants. This is also evident in the unfolding transition midpoints, T* and c_{1/2}, which are lower for gain-of-function and higher for loss-of-function variants. The T* and c_{1/2} values are also positively correlated so that a change in thermal stability is paralleled by a comparable change in urea stability (see FIG. 26). These results remain in general agreement with the inventor's previous conclusions (17 and supra). The Native-Like variants, A1437T and R1341Q, also have low stability, but because they are less structured, some regions of the A1 domain are likely to be partially unfolded. Intrinsic protein FL is generally not sensitive to conformational differences between natively structured and Native-Like conformations because the FL intensities are similar in both pre- and postthermal unfolding transitions. However, ANS FL does discriminate between these conformations, because the mutations result in exposure of new hydrophobic groups in the A1 domain.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

### Example 1

Auton M, Sowa K E, Behymer M, Cruz M A (2012) The N-terminal flanking region of A1 domain in von Willebrand factor stabilizes the structure of the A1A2A3 complex and modulates platelet activation under shear stress. The Journal of biological chemistry 287:14579-14585.
Bonthron D T, Handin R I, Kaufman R J, Wasley L C, On E C, Mitsock L M, Ewenstein B, Loscalzo J, Ginsburg D, Orkin S H (1986) Structure of pre-pro-von Willebrand factor and its expression in heterologous cells. Nature 324:270-273.
Sadler J E (1998) Biochemistry and genetics of von Willebrand factor. Annu Rev. Biochem. 67:395-424.
Savage B, Saldivar E, Ruggeri Z M (1996) Initiation of platelet adhesion by arrest onto fibrinogen or translocation on von Willebrand factor. Cell 84:289-297.
Fujimura Y, Titani K, Holland L Z, Russell S R, Roberts J R, Elder J H, Ruggeri Z M, Zimmerman T S (1986) von Willebrand factor. A reduced and alkylated 52/48-kDa fragment beginning at amino acid residue 449 contains the domain interacting with platelet glycoprotein Ib. J Biol Chem 261:381-385.
Fujimura Y, Titani K, Holland L Z, Roberts J R, Kostel P, Ruggeri Z M, Zimmerman T S (1987) A heparin-binding domain of human von Willebrand factor. Characterization and localization to a tryptic fragment extending from amino acid residue Val-449 to Lys-728. J Biol Chem 262:1734-1739.
Pietu G, Meulien P, Cherel G, Diaz J, Baruch D, Courtney M, Meyer D (1989) Production in Escherichia coli of a biologically active subfragment of von Willebrand factor corresponding to the platelet glycoprotein Ib, collagen and heparin binding domains. Biochem. Biophys. Res. Commun. 164:1339-1347.
Sugimoto M, Ricca G, Hrinda M E, Schreiber A B, Searfoss G H, Bottini E, Ruggeri Z M (1991) Functional modulation of the isolated glycoprotein Ib binding domain of von Willebrand factor expressed in Escherichia coli. Biochemistry 30:5202-5209.
Gralnick H R, Williams S, McKeown L, Kramer W, Krutzsch H, Gorecki M, Pinet A, Garfinkel L I (1992) A monomeric von Willebrand factor fragment, Leu-504-Lys-728, inhibits von Willebrand factor interaction with glycoprotein Ib-IX. Proc. Natl. Acad. Sci. U.S.A. 89:7880-7884.
Azuma H, Dent J A, Sugimoto M, Ruggeri Z M, Ware J (1991) Independent assembly and secretion of a dimeric adhesive domain of von Willebrand factor containing the glycoprotein Ib-binding site. J Biol Chem 266:12342-12347.
Cruz M A, Handin R I, Wise R J (1993) The interaction of the von Willebrand factor-A1 domain with platelet glycoprotein Ib/IX. The role of glycosylation and disulfide bonding in a monomeric recombinant A1 domain protein. J Biol Chem 268:21238-21245.
Arya M, Anvari B, Romo G M, Cruz M A, Dong J F, McIntire L V, Moake J L, Lopez J A (2002) Ultralarge multimers of von Willebrand factor form spontaneous high-strength bonds with the platelet glycoprotein Ib-IX complex: studies using optical tweezers. Blood 99:3971-3977.
Arya M, Kolomeisky A B, Romo G M, Cruz M A, Lopez J A, Anvari B (2005) Dynamic force spectroscopy of glycoprotein Ib-IX and von Willebrand Factor. Biophys. J. 88:4391-4401.
Coburn L A, Damaraju V S, Dozic S, Eskin S G, Cruz M A, McIntire L V (2011) GPIbα-vWF rolling under shear stress shows differences between type 2B and 2M von Willebrand disease. Biophys. J. 100:304-312.
Yago T, Lou J, Wu T, Yang J, Miner J J, Coburn L, Lopez J A, Cruz M A, Dong J F, McIntire L V, et al. (2008) Platelet glycoprotein Ibα forms catch bonds with human WT vWF but not with type 2B von Willebrand disease vWF. J. Clin. Invest. 118:3195-3207.
Auton M, Zhu C, Cruz M A (2010) The Mechanism of VWF-Mediated Platelet GPIbα Binding. Biophys. J. 99:1192-1201.
Auton M, Sedlak E, Marek J, Wu T, Zhu C, Cruz M A (2009) Changes in thermodynamic stability of von Willebrand factor differentially affect the force-dependent binding to platelet GPIbα. Biophys. J. 97:618-627.
Emsley J, Cruz M, Handin R, Liddington R (1998) Crystal structure of the von Willebrand Factor A1 domain and implications for the binding of platelet glycoprotein Ib. J Biol Chem 273:10396-10401.
Fukuda K, Doggett T A, Bankston L A, Cruz M A, Diacovo T G, Liddington R C (2002) Structural basis of von Willebrand factor activation by the snake toxin botrocetin. Structure 10:943-950.
Kim J, Zhang C Z, Zhang X, Springer T A (2010) A mechanically stabilized receptor-ligand flex-bond important in the vasculature. Nature 466:992-995.
Miyata S, Ruggeri Z M (1999) Distinct structural attributes regulating von Willebrand factor A1 domain interaction with platelet glycoprotein Ibα under flow. J Biol Chem 274:6586-6593.
Celikel R, Ruggeri Z M, Varughese K I (2000) von Willebrand factor conformation and adhesive function is modulated by an internalized water molecule. Nat Struct Biol 7:881-884.
Huizinga E G, Tsuji S, Romijn R A, Schiphorst M E, de Groot P G, Sixma J J, Gros P (2002) Structures of glycoprotein Ibα and its complex with von Willebrand factor A1 domain. Science 297:1176-1179.
Dumas J J, Kumar R, McDonagh T, Sullivan F, Stahl M L, Somers W S, Mosyak L (2004) Crystal structure of the wild-type von Willebrand factor A1-glycoprotein Ibα complex reveals conformation differences with a complex bearing von Willebrand disease mutations. J Biol Chem 279:23327-23334.
Mohri H, Fujimura Y, Shima M, Yoshioka A, Houghten R A, Ruggeri Z M, Zimmerman T S (1988) Structure of the von Willebrand factor domain interacting with glycoprotein Ib. J Biol Chem 263:17901-17904.
Fujimura Y, Usami Y, Titani K, Niinomi K, Nishio K, Takase T, Yoshioka A, Fukui H (1991) Studies on anti-von Willebrand factor (vWF) monoclonal antibody NMC-4, which inhibits both ristocetin- and botrocetin-induced vWF binding to platelet glycoprotein Ib. Blood 77:113-120.
Celikel R, Varughese K I, Madhusudan, Yoshioka A, Ware J, Ruggeri Z M (1998) Crystal structure of the von Willebrand factor A1 domain in complex with the function blocking NMC-4 Fab. Nat Struct Biol 5:189-194.
Ulrichts H et al. (2006) Shielding of the A1 domain by the D′D3 domains of von Willebrand factor modulates its interaction with platelet glycoprotein Ib-IX-V. J Biol Chem 281:4699-4707.
Tornai I, Arnout J, Deckmyn H, Peerlinck K, Vermylen J (1993) A monoclonal antibody recognizes a von Willebrand factor domain within the amino-terminal portion of the subunit that modulates the function of the glycoprotein IB- and IIB/IIIA-binding domains. J. Clin. Invest. 91:273-282.
Ulrichts H, Harsfalvi J, Bene L, Matko J, Vermylen J, Ajzenberg N, Baruch D, Deckmyn H, Tornai I (2004) A monoclonal antibody directed against human von Willebrand factor induces type 2B-like alterations. Journal of Thrombosis and Haemostasis 2:1622-1628.
Bohm G, Muhr R, Jaenicke R (1992) Quantitative analysis of protein far UV circular dichroism spectra by neural networks. Protein Eng. 5:191-195.
Auton M, Sowa K E, Smith S M, Sedlak E, Vijayan K V, Cruz M A (2010) Destabilization of the A1 domain in von Willebrand factor dissociates the A1A2A3 tri-domain and provokes spontaneous binding to glycoprotein Ibα and platelet activation under shear stress. J Biol Chem 285:22831-22839.
Morales L D, Martin C, Cruz M A (2006) The interaction of von Willebrand factor-A1 domain with collagen: mutation G1324S (type 2M von Willebrand disease) impairs the conformational change in A1 domain induced by collagen. J Thromb Haemost 4:417-425.
Cruz M A, Diacovo T G, Emsley J, Liddington R, Handin R I (2000) Mapping the glycoprotein Ib-binding site in the von Willebrand factor A1 domain. J Biol Chem 275:19098-19105.
Auton M, Cruz M A, Moake J (2007) Conformational Stability and Domain Unfolding of the von Willebrand actor A Domains. J. Mol. Biol. 366:986-1000.
Pace C N, Vajdos F, Fee L, Grimsley G, Gray T (1995) How to measure and predict the molar absorption coefficient of a protein. Protein Sci. 4:2411-2423.
Savitzky A, Golay M J E (1964) Smoothing and Differentiation of Data by Simplifies Least Squares Procedures. Anal Chem 34:1627-1639.
Atkins P W Chapter 25: The rates of chemical reactions. In: Physical Chemistry. 5 ed. New York: W. H. Freeman and Company; 1994. pp. 861-897.
Lyubarev A E, Kurganov B I (1998) Modeling of irreversible thermal protein denaturation at varying temperature. I. The model involving two consecutive irreversible steps. Biochemistry (Mosc) 63:434-440.
Kurganov B I, Lyubarev A E, Sanchez-Ruiz J M, Shnyrov V L (1997) Analysis of differential scanning calorimetry data for proteins. Criteria of validity of one-step mechanism of irreversible protein denaturation. Biophys. Chem. 69:125-135.
Rainville E D, Bedient P E Chapter 2—Equations of order one. In: Elementary Differential Equations. 7 ed. New York: Macmillan Publishing Company; pp. 17-46.
Savitzky A, Golay M J E (1964) Smoothing and Differentiation of Data by Simplifies Least Squares Procedures. Anal Chem 34:1627-1639.
Atkins P W Chapter 25: The rates of chemical reactions. In: Physical Chemistry. 5 ed. New York: W. H. Freeman and Company; 1994. pp. 861-897.
Kurganov B I, Lyubarev A E, Sanchez-Ruiz J M, Shnyrov V L (1997) Analysis of differential scanning calorimetry data for proteins. Criteria of validity of one-step mechanism of irreversible protein denaturation. Biophys. Chem. 69:125-135.
Lyubarev A E, Kurganov B I (1998) Modeling of irreversible thermal protein denaturation at varying temperature. I. The model involving two consecutive irreversible steps. Biochemistry (Mosc) 63:434-440.
Rainville E D, Bedient P E Chapter 2—Equations of order one. In: Elementary Differential Equations. 7 ed. New York: Macmillan Publishing Company; pp. 17-46.
Bohm G, Muhr R, Jaenicke R (1992) Quantitative analysis of protein far UV circular dichroism spectra by neural networks. Protein Eng. 5:191-195.

### REFERENCES

### Example 2

Woods A I, Sanchez-Luceros A, Kempfer A C, et al. C1272F: a novel type 2A von Willebrand's disease mutation in A1 domain; its clinical significance. Haemophilia. 2012; 18(1):112-116.
Penas N, Perez A, Gonzalez-Boullosa R, Batlle J. C1272S: a new candidate mutation in type 2A von Willebrand disease that disrupts the disulfide loop responsible for the interaction of vWF with platelet GP Ib-IX. Am J Hematol. 2004; 75(2):73-77.
Meyer D, Fressinaud E, Gaucher C, et al. Gene defects in 150 unrelated French cases with type 2 von Willebrand disease: from the patient to the gene. INSERM Network on Molecular Abnormalities in von Willebrand Disease. Thromb. Haemost. 1997; 78(1):451-456.
Lavergne J M, De Paillette L, Bahnak B R, et al. Defects in type HA von Willebrand disease: a cysteine 509 to arginine substitution in the mature von Willebrand factor disrupts a disulphide loop involved in the interaction with platelet glycoprotein Ib-IX. Br J Haematol. 1992; 82(1):66-72.
Azuma H, Hayashi T, Dent J A, Ruggeri Z M, Ware J. Disulfide bond requirements for assembly of the platelet glycoprotein Ib-binding domain of von Willebrand factor. J Biol Chem. 1993; 268(4):2821-2827.
Cruz M A, Handin R I, Wise R J. The interaction of the von Willebrand factor-A1 domain with platelet glycoprotein Ib/IX. The role of glycosylation and disulfide bonding in a monomeric recombinant A1 domain protein. J Biol Chem. 1993; 268(28):21238-21245.
Andrews R K, Gorman J J, Booth W J, et al. Cross-linking of a monomeric 39/34-kDa dispase fragment of von Willebrand factor (Leu-480/Val-481-Gly-718) to the N-terminal region of the α-chain of membrane glycoprotein Ib on intact platelets with bis(sulfosuccinimidyl) suberate. Biochemistry. 1989; 28(21):8326-8336.
Miura S, Fujimura Y, Sugimoto M, et al. Structural elements influencing von Willebrand factor (vWF) binding affinity for platelet glycoprotein Ib within a dispase-digested vWF fragment. Blood. 1994; 84(5):1553-1558.
Siguret V, Ribba A S, Christophe O, et al. Characterization of recombinant von Willebrand factors mutated on cysteine 509 or 695. Thromb. Haemost. 1996; 76(3):453-459.
Miyata S, Goto S, Federici A B, Ware J, Ruggeri Z M. Conformational changes in the A1 domain of von Willebrand factor modulating the interaction with platelet glycoprotein Ibα. J Biol Chem. 1996; 271(15):9046-9053.
Miyata S, Ruggeri Z M. Distinct structural attributes regulating von Willebrand factor A1 domain interaction with platelet glycoprotein Ibα under flow. J Biol Chem. 1999; 274(10):6586-6593.
Auton M, Zhu C, Cruz M A. The Mechanism of VWF-Mediated Platelet GPIbα Binding. Biophys. J. 2010; 99(4):1192-1201.
Auton M, Sedlak E, Marek J, et al. Changes in thermodynamic stability of von Willebrand factor differentially affect the force-dependent binding to platelet GPIbα. Biophys. J. 2009; 97(2):618-627.
Auton M, Cruz M A, Moake J. Conformational Stability and Domain Unfolding of the von Willebrand Factor A Domains. J. Mol. Biol. 2007; 366(3):986-1000.
Kim J, Zhang C Z, Zhang X, Springer T A. A mechanically stabilized receptor-ligand flex-bond important in the vasculature. Nature. 2010; 466(7309):992-995.
Thomas W, Forero M, Yakovenko O, et al. Catch-bond model derived from allostery explains force-activated bacterial adhesion. Biophys. J. 2006; 90(3):753-764.
Tischer A, Cruz M A, Auton M. The linker between the D3 and A1 domains of vWF suppresses A1-GPIbα catch bonds by site specific binding to the A1 domain. Protein Sci. 2013; 22(8): 1049-1059.
Nozaki Y, Tanford C. The solubility of amino acids, diglycine, and triglycine in aqueous guanidine hydrochloride solutions. J Biol Chem. 1970; 245(7):1648-1652.
Thomas W. Catch bonds in adhesion. Annu Rev Biomed Eng. 2008; 10:39-57.
Thomas W E, Vogel V, Sokurenko E. Biophysics of catch bonds. Annu Rev Biophys. 2008; 37:399-416.
Coburn L A, Damaraju V S, Dozic S, et al. GPIbα-vWF rolling under shear stress shows differences between type 2B and 2M von Willebrand disease. Biophys. J. 2011; 100(2):304-312.
Yago T, Lou J, Wu T, et al. Platelet glycoprotein Ibα forms catch bonds with human WT vWF but not with type 2B von Willebrand disease vWF. J. Clin. Invest. 2008; 118(9):3195-3207.
Baldwin R L, Rose G D. Molten globules, entropy-driven conformational change and protein folding. Current Opinion in Structural Biology. 2013; 23(1):4-10.
Semisotnov G V, Rodionova N A, Razgulyaev O I, et al. Study of the “molten globule” intermediate state in protein folding by a hydrophobic fluorescent probe. Biopolymers. 1991; 31(1):119-128.
Uversky V N. Natively unfolded proteins: a point where biology waits for physics. Protein Sci. 2002; 11:739-756.
Lim W K, R\″osgen J, Englander S W. Urea, but not guanidinium, destabilizes proteins by forming hydrogen bonds to the peptide group. Proc Natl Acad Sci USA 2009; 106(8):2595-2600.
Huizinga E G et al. Structures of glycoprotein Ibα and its complex with von Willebrand factor A1 domain. Science. 2002; 297:1176-1179.
Uff S, Clemetson J M, Harrison T, Clemetson K J, Emsley J. Crystal structure of the platelet glycoprotein Ib(α) N-terminal domain reveals an unmasking mechanism for receptor activation. J Biol Chem. 2002; 277:35657-35663.
McEwan P A, Andrews R K, Emsley J. Glycoprotein Ib inhibitor complex structure reveals a combined steric and allosteric mechanism of von Willebrand factor antagonism. Blood. 2009; 114:4883-4885.
Prajapati R S, Indu S, Varadarajan R. Identification and Thermodynamic Characterization of Molten Globule States of Periplasmic Binding Proteins. Biochemistry. 2007; 46(36):10339-10352.
Vassilenko K S, Uversky V N. Native-like secondary structure of molten globules. Biochim. Biophys. Acta. 2002; 1594(1):168-177.
Redfield C, Schulman B A, Milhollen M A, Kim P S, Dobson C M. Alpha-lactalbumin forms a compact molten globule in the absence of disulfide bonds. Nat Struct Biol. 1999; 6(10):948-952.
Molten globule monomer to condensed dimer: role of disulfide bonds in platelet factor-4 folding and subunit association. Biochemistry. 1992; 31(48):12255-12265.
Tjernberg P, Castaman G, Vos H L, Bertina R M, Eikenboom J C. Homozygous C2362F von Willebrand factor induces intracellular retention of mutant von Willebrand factor resulting in autosomal recessive severe von Willebrand disease. Br J Haematol. 2006; 133(4):409-418.
Hommais A, Stepanian A, Fressinaud E, et al. Mutations C1157F and C1234W of von Willebrand factor cause intracellular retention with defective multimerization and secretion. J Thromb Haemost. 2006; 4(1):148-157.
Casari C, Lenting P J, Wohner N. Clearance of von Willebrand factor. Journal of Thrombosis and Haemostasis, 2013.
Casonato A, Gallinaro L, Cattini M G, et al. Reduced survival of type 2B von Willebrand factor, irrespective of large multimer representation or thrombocytopenia. Haematologica. 2010; 95(8):1366-1372.
Rayes J, Hollestelle M J, Legendre P, et al. Mutation and ADAMTS13-dependent modulation of disease severity in a mouse model for von Willebrand disease type 2B. Blood. 2010; 115(23):4870-4877.
Rayes J, Hommais A, Legendre P, et al. Effect of von Willebrand disease type 2B and type 2M mutations on the susceptibility of von Willebrand factor to ADAMTS-13—RAYES—2007—Journal of Thrombosis and Haemostasis—Wiley Online Library. J Thromb Haemost. 2007; 5(2):321-328.
Hassenpflug W A, Budde U, Obser T, et al. Impact of mutations in the von Willebrand factor A2 domain on ADAMTS13-dependent proteolysis. Blood. 2006; 107(6):2339-2345.
Lenting P J, van Schooten C J M, Denis C V. Clearance mechanisms of von Willebrand factor and factor VIII. J Thromb Haemost. 2007; 5:1353-1360.
Badirou I et al. von Willebrand factor clearance does not involve proteolysis by ADAMTS-13. J Thromb Haemost. 2010; 8:2338-2340.
Schooten C J, Tjernberg P, Westein E, et al. Cysteine-mutations in von Willebrand factor associated with increased clearance. J Thromb Haemost. 2005; 3(10):2228-2237.
Lippincott-Schwartz J, Roberts T H, Hirschberg K. Secretory Protein Trafficking and Ogranelle Dynamics in Living Cells. Annu Rev. Cell Dev. Biol. 2000; 16:557-589.
Kincaid M M, Cooper A A. Misfolded proteins traffic from the endoplasmic reticulum (ER) due to ER export signals. Mol. Biol. Cell. 2007; 18(2):455-463.
Pace C N, Vajdos F, Fee L, Grimsley G, Gray T. How to measure and predict the molar absorption coefficient of a protein. Protein Sci. 1995; 4(11):2411-2423.
Motono C, Yamagishi A, Oshima T. Urea-Induced Unfolding and Conformational Stability of 3-Isopropylmalate Dehydrogenase from the Thermophile Thermus thermophilus and Its Mesophilic Counterpart from Escherichia coli. Biochemistry. 1999; 38(4):1332-1337.
Savitzky A, Golay M J E. Smoothing and Differentiation of Data by Simplifies Least Squares Procedures. Anal Chem. 1964; 34:1627-1639.
Lakowicz J R. Quenching of Fluorescence. Principles of Fluorescence Spectroscopy. 2006; (8):277-330.
Bohm G, Muhr R, Jaenicke R Quantitative analysis of protein far UV circular dichroism spectra by neural networks. Protein Eng. 1992; 5:191-195.

### REFERENCES

### Example 3

1. Rodeghiero, F., G. Castaman, and E. Dini. 1987. Epidemiological investigation of the prevalence of von Willebrand's disease. Blood. 69:454-459.
2. Lillicrap, D. 2013. von Willebrand disease: advances in pathogenetic understanding, diagnosis and therapy. Blood. dx.doi.org/10.1182/blood-2013-06-498303.
3. Peyvandi, F., T. Kunicki, and D. Lillicrap. 2013. Genetic sequence analysis of inherited bleeding diseases. Blood. 122:3423-3431.
4. Weiss, H. J., V. T. Turitto, and H. R. Baumgartner. 1978. Effect of shear rate on platelet interaction with subendothelium in citrated and native blood. I. Shear rate-dependent decrease of adhesion in von Willebrand's disease and the Bernard-Soulier syndrome. J. Lab. Clin. Med. 92:750-764.
5. Sadler, J. E., B. B. Shelton-Inloes, E. W. Davie. 1985. Cloning and characterization of two cDNAs coding for human von Willebrand factor. Proc. Natl. Acad. Sci. USA. 82:6394-6398.
6. Titani, K., S. Kumar, K. Fujikawa. 1986. Amino acid sequence of human von Willebrand factor. Biochemistry. 25:3171-3184.
7. Mancuso, D. J., E. A. Tuley, J. E. Sadler. 1989. Structure of the gene for human von Willebrand factor. J. Biol. Chem. 264:19514-19527.
8. Randi, A. M., S. Jorieux, J. E. Sadler. 1992. Recombinant von Willebrand Factor Arg578/Gln. A type IIB von Willebrand disease mutation affects binding to glycoprotein Ib but not to collagen or heparin. J. Biol. Chem. 267:21187-21192.
9. Cooney, K. A., W. C. Nichols, D. Ginsburg. 1991. The molecular defect in type JIB von Willebrand disease. Identification of four potential missense mutations within the putative GpIb binding domain. J. Clin. Invest. 87:1227-1233.
10. Rabinowitz, I., E. A. Tuley, J. E. Sadler. 1992. von Willebrand disease type B: a missense mutation selectively abolishes ristocetin-induced von Willebrand factor binding to platelet glycoprotein Ib. Proc. Natl. Acad. Sci. USA. 89:9846-9849.
11. Cruz, M. A., T. G. Diacovo, R. I. Handin. 2000. Mapping the glycoprotein Ib-binding site in the von Willebrand factor A1 domain. J. Biol. Chem. 275:19098-19105.
12. Rastegar-Lari, G., N. Ajzenberg, D. Baruch. 2001. Defect of heparin binding in plasma and recombinant von Willebrand factor with type 2 von Willebrand disease mutations. Thromb. Haemost. 86:1459-1465.
13. Stepanian, A., A. S. Ribba, D. Meyer. 2003. A new mutation, S1285F, within the A1 loop of von Willebrand factor induces a conformational change in A1 loop with abnormal binding to platelet GPIb and botrocetin causing type 2M von Willebrand disease. Br. J. Haematol. 120:643-651.
14. Miyata, S., S. Goto, Z. M. Ruggeri. 1996. Conformational changes in the A1 domain of von Willebrand factor modulating the interaction with platelet glycoprotein Iba. J. Biol. Chem. 271:9046-9053.
15. Miyata, S., and Z. M. Ruggeri. 1999. Distinct structural attributes regulating von Willebrand factor A1 domain interaction with platelet glycoprotein Iba under flow. J. Biol. Chem. 274:6586-6593.
16. Auton, M., M. A. Cruz, and J. Moake. 2007. Conformational stability and domain unfolding of the von Willebrand factor A domains. J. Mol. Biol. 366:986-1000.
17. Auton, M., C. Zhu, and M. A. Cruz. 2010. The mechanism of vWF-mediated platelet GPIba binding. Biophys. J. 99:1192-1201.
18. Auton, M., E. Sedlak, M. A. Cruz. 2009. Changes in thermodynamic stability of von Willebrand factor differentially affect the force-dependent binding to platelet GPIba. Biophys. J. 97:618-627.
19. Tischer, A., P. Madde, M. Auton. 2014. A molten globule intermediate of the von Willebrand factor A1 domain firmly tethers platelets under shear flow. Proteins. 82:867-878.
20. Tischer, A., M. A. Cruz, and M. Auton. 2013. The linker between the D3 and A1 domains of vWF suppresses A1-GPIba catch bonds by site-specific binding to the A1 domain. Protein Sci. 22:1049-1059.
21. Tischer, A., and M. Auton. 2013. Urea-temperature phase diagrams capture the thermodynamics of denatured state expansion that accompany protein unfolding. Protein Sci. 22:1147-1160.
22. Blancas-Mejia, L. M., A. Tischer, M. Ramirez-Alvarado. 2014. Kinetic control in protein folding for light chain amyloidosis and the differential effects of somatic mutations. J. Mol. Biol. 426:347-361.
23. Yago, T., J. Lou., C. Zhu. 2008. Platelet glycoprotein Iba forms catch bonds with human WT vWF but not with type 2B von Willebrand disease vWF. J. Clin. Invest. 118:3195-3207.
24. Auton, M., K. E. Sowa, M. A. Cruz. 2010. Destabilization of the A1 domain in von Willebrand factor dissociates the A1A2A3 tri-domain and provokes spontaneous binding to glycoprotein Iba and platelet activation under shear stress. J. Biol. Chem. 285:22831-22839.
25. Kim, J., C. Z. Zhang, T. A. Springer. 2010. A mechanically stabilized receptor-ligand flex-bond important in the vasculature. Nature. 466:992-995.
26. Baronciani, L., A. B. Federici, P. M. Mannucci. 2005. Expression studies on a novel type 2B variant of the von Willebrand factor gene (R13080 characterized by defective collagen binding. J. Thromb. Haemost. 3:2689-2694.
27. Federici, A. B., P. M. Mannucci, P. G. De Groot. 2009. Clinical and molecular predictors of thrombocytopenia and risk of bleeding in patients with von Willebrand disease type 2B: a cohort study of 67 patients. Blood. 113:526-534.
28. Favaloro, E. J. 2011. Diagnosis and classification of von Willebrand disease: a review of the differential utility of various functional von Willebrand factor assays. Blood Coagul. Fibrinolysis. 22:553-564.
29. Hayes, T. E., J. T. Brandt, M. T. Cunningham. 2006. External peer review quality assurance testing in von Willebrand disease: the recent experience of the United States College of American Pathologists proficiency testing program. Semin. Thromb. Hemost. 32:499-504.
30. Kitchen, S., I. Jennings, F. E. Preston. 2006. Laboratory tests for measurement of von Willebrand factor show poor agreement among different centers: results from the United Kingdom National External Quality Assessment Scheme for Blood Coagulation. Semin. Thromb. Hemost. 32:492-498.
31. Meijer, P., and F. Haverkate. 2006. An external quality assessment program for von Willebrand factor laboratory analysis: an overview from the European concerted action on thrombosis and disabilities foundation. Semin. Thromb. Hemost. 32:485-491.
32. Nichols, W. L., M. E. Rick, M. Weinstein. 2009. Clinical and laboratory diagnosis of von Willebrand disease: a synopsis of the 2008 NHLBI/NIH guidelines. Am. J. Hematol. 84:366-370.
33. Gangarosa, E. J., N. S. Landerman, E. G. Herndon, Jr. 1958. Hematologic complications arising during ristocetin therapy; relation between dose and toxicity. N. Engl. J. Med. 259:156-161.
34. Flood, V. H., K. D. Friedman, R. R. Montgomery. 2009. Limitations of the ristocetin cofactor assay in measurement of von Willebrand factor function. J. Thromb. Haemost. 7:1832-1839.
35. Howard, M. A., and B. G. Firkin. 1971. Ristocetin—a new tool in the investigation of platelet aggregation. Thromb. Diath. Haemorrh. 26:362-369.
36. Chandler, W. L., E. I. B. Peerschke, P. Meijer. 2011. von Willebrand factor assay proficiency testing. The North American Specialized Coagulation Laboratory Association experience. Am. J. Clin. Pathol. 135:862-869.
37. Howard, M. A., R. J. Sawers, and B. G. Firkin. 1973. Ristocetin: a means of differentiating von Willebrand's disease into two groups. Blood. 41:687-690.
38. Weiss, H. J., L. W. Hoyer, J. Rogers. 1973. Quantitative assay of a plasma factor deficient in von Willebrand's disease that is necessary for platelet aggregation. Relationship to factor VIII procoagulant activity and antigen content. J. Clin. Invest. 52:2708-2716.
39. Girma, J. P., Y. Takahashi, D. Meyer. 1990. Ristocetin and botrocetin involve two distinct domains of von Willebrand factor for binding to platelet membrane glycoprotein Ib. Thromb. Haemost. 64:326-332.
40. De Luca, M., D. A. Facey, M. C. Berndt. 2000. Structure and function of the von Willebrand factor A1 domain: analysis with monoclonal antibodies reveals distinct binding sites involved in recognition of the platelet membrane glycoprotein Ib-IX-V complex and ristocetin-dependent activation. Blood. 95:164-172.
41. Scott, J. P., R. R. Montgomery, and G. S. Retzinger. 1991. Dimeric ristocetin flocculates proteins, binds to platelets, and mediates von Willebrand factor-dependent agglutination of platelets. J. Biol. Chem. 266:8149-8155.
42. Zwaginga, J. J., K. S. Sakariassen, J. W. Heemskerk. 2007. Can blood flow assays help to identify clinically relevant differences in von Willebrand factor functionality in von Willebrand disease types 1-3? J. Thromb. Haemost. 5:2547-2549.
43. Favaloro, E. J. 2008. Can blood flow assays help to identify clinically relevant differences in von Willebrand factor functionality in von Willebrand disease types 1-3? J. Thromb. Haemost. 6:545-546.
44. Emsley, J., M. Cruz, R. Liddington. 1998. Crystal structure of the von Willebrand factor A1 domain and implications for the binding of platelet glycoprotein Ib. J. Biol. Chem. 273:10396-10401.
45. Fukuda, K., T. A. Doggett, R. C. Liddington. 2002. Structural basis of von Willebrand factor activation by the snake toxin botrocetin. Structure. 10:943-950.
46. Huizinga, E. G., S. Tsuji, P. Gros. 2002. Structures of glycoprotein Iba and its complex with von Willebrand factor A1 domain. Science. 297:1176-1179.
47. Dumas, J. J., R. Kumar, L. Mosyak. 2004. Crystal structure of the wild-type von Willebrand factor A1-glycoprotein Iba complex reveals conformation differences with a complex bearing von Willebrand disease mutations. J. Biol. Chem. 279:23327-23334.
48. Celikel, R., Z. M. Ruggeri, and K. I. Varughese. 2000. von Willebrand factor conformation and adhesive function is modulated by an internalized water molecule. Nat. Struct. Biol. 7:881-884.
49. Chen, W., J. Lou, and C. Zhu. 2009. Molecular dynamics simulated unfolding of von Willebrand factor A domains by force. Cell. Mol. Bioeng. 2:75-86.
50. Chen, W., J. Lou, and C. Zhu. 2010. Simulated thermal unfolding of the von Willebrand factor A domains. Cell. Mol. Bioeng. 3:117-127.
51. Interlandi, G., and W. Thomas. 2010. The catch bond mechanism between von Willebrand factor and platelet surface receptors investigated by molecular dynamics simulations. Proteins. 78:2506-2522.
52. Li, J., L. Zhang, and Y. Sun. 2012. Molecular basis of the initial platelet adhesion in arterial thrombosis: molecular dynamics simulations. J. Mol. Graph. Model. 37:49-58.
53. Liu, G., Y. Fang, and J. Wu. 2013. A mechanism for localized dynamics-driven affinity regulation of the binding of von Willebrand factor to platelet glycoprotein Iba. J. Biol. Chem. 288:26658-26667.
54. Lillicrap, D. 2009. Genotype/phenotype association in von Willebrand disease: is the glass half full or empty? J. Thromb. Haemost. 7 (Suppl 1): 65-70.
55. Bonnefoy, A., R. A. Romijn, M. F. Hoylaerts. 2006. von Willebrand factor A1 domain can adequately substitute for A3 domain in recruitment of flowing platelets to collagen. J. Thromb. Haemost. 4:2151-2161.
56. Rayes, J., A. Hommais, J. P. Girma. 2007. Effect of von Willebrand disease type 2B and type 2M mutations on the susceptibility of von Willebrand factor to ADAMTS-13. J. Thromb. Haemost. 5:321-328.
57. Schrank, T. P., D. W. Bolen, and V. J. Hilser. 2009. Rational modulation of conformational fluctuations in adenylate kinase reveals a local unfolding mechanism for allostery and functional adaptation in proteins. Proc. Natl. Acad. Sci. USA. 106:16984-16989.
58. Springer, T. A. 2011. Biology and physics of von Willebrand factor concatamers. J. Thromb. Haemost. 9 (Suppl 1):130-143.

What is claimed is:

1. An apparatus for measuring cell or platelet adhesion comprising:
a rheological shear flow surface coated with an agent that provides cell or platelet adhesion;
a detector to track the transit of cells or platelets on the surface under shear flow; and
a processor that calculates both a pause time and a roll time of platelets, wherein the processor determines a median and a mean pause time and a median and a mean roll time of cells or platelets and compares them to cells or platelets having at least one of: no cell adhesion dysfunction, a mild adhesion dysfunction, or a severe adhesion dysfunction, between a cell surface adhesion molecule on the cell or platelet and the agent, wherein the processor provides a real-time, quantitative measurement of a dynamic range of cell or platelet adhesion.

2. The apparatus of claim 1, wherein the cell surface adhesion molecule is von Willebrand Factor, an ICAM, a VCAM, a Lectin, an Integrin, a collagen, a fibrinogen, a subendothelial membrane protein, glycoprotein Ib(β), glycoproteinIb(α), glycoprotein Ib-IX-V complex, glycoprotein IIbIIIa, glyprotein VI, major histocompatibility complex, integrin α2 β1, or immunoglobin.

3. The apparatus of claim 1, wherein the cell surface adhesion molecule is von Willebrand Factor protein comprising one or more point mutations in the A1, A2, A3, B, or C domains.

4. The apparatus of claim 1, wherein the surface is coated with a mutant human von Willebrand factor protein.

5. The apparatus of claim 1, wherein the cells or platelets are obtained from a suspect suspected of having von Willebrand disease, platelet type von Willebrand disease, acquired von Willebrand syndrome, hypertrophic cardiomyopathy, Bernard-Soulier syndrome, Glanzmann's thrombasthenia thrombocytopenia, or various autoimmune coagulation disorders.

6. The apparatus of claim 1, wherein the cells or platelets are obtained from a suspect suspected of having coagulopathies associated with left ventricular assist device implantation.

7. The apparatus of claim 1, wherein the surface is in a multifluidic chamber.

8. The apparatus of claim 1, wherein the surface is coated with a truncated human von Willebrand factor fusion protein that comprises a human von Willebrand factor platelet adhesion domain and a recombinant binding domain.

9. The apparatus of claim 1, wherein the processor calculates the dynamic range of platelet factor to distinguish between two or more low adhesion cell or platelet binding dysfunctions that is statistically significant.

10. The apparatus of claim 1, wherein the apparatus distinguishes between different low adhesion cell or platelet binding dysfunctions that is statistically significant.

11. The apparatus of claim 1, wherein the cells are selected from T cells, B cells, macrophages, neutrophils, basophils, or eosinophils.

12. The apparatus of claim 1, wherein the agent comprises an ethylene glycol polymer bound to the surface and a bivalent cation to provide a consistent binding surface for a His-tag or equivalent bivalent cation binding peptide or polypeptide that is removably attached with high affinity to the bivalent cation.

13. The apparatus of claim 1, wherein between 100 to 1000, 1000 to 10,000, 10,000 to 15,000, 25,000 to 50,000, 50 to 75,000, 75,000 to 100,000, 100 to 100,000, 1000 to 90,000, 20,000 to 80,000, 30,000 to 70,000, 40,000 to 60,000 or more than 100,000 cells or platelets are imaged and processed to determine the median and the mean roll time of cells or platelets is determined to calculate the level of adhesion of the cells or platelets to the agent on the surface.

14. The apparatus of claim 1, wherein the dynamic range is in the low adhesion level.

15. The apparatus of claim 1, wherein the dynamic range is in the low adhesion level and provides differentiation between various low adhesion point mutants in von Willebrand disease.

16. The apparatus of claim 1, wherein the processor calculates the adhesion and determines if the von Willebrand Factor is natively structured, nativelike 2M, nativelike 2B, molten globule 2M or molten globule 2B.

17. The apparatus of claim 1, wherein the processor distinguishes between a native von Willebrand Factor A1 domain and one or more of the following mutations: G1324S (2M); A1437T (2M); R1308L (2B); R1341Q (2B); R1306Q (2B); I1372S (2B); I1309V (2B); F1369I (2M); E1359K (2M); I1425F (2M); S1285F (2M); R1374H (2M); H1268D (2B); or V1316M.

18. A method of using a real-time quantitative measurement of a dynamic range of cell or platelet function to select a treatment comprising:
obtaining a cell or platelet sample from a subject suspected of having a dysfunction in cell or platelet adhesion;
flowing the cell or platelet sample over a rheological shear flow surface, wherein the surface is coated with an agent that provides cell or platelet binding;
measuring the transit of the cell or platelet sample on the surface under shear flow;
calculating both a pause time and a roll time of the cells or platelets obtained from a subject suspected of having a cell or platelet dysfunction, wherein the processor determines a median and a mean pause time and a median and a mean roll time of cells or platelets and compares them to cells or platelets having at least one of: no cell or platelet adhesion dysfunction, a mild adhesion dysfunction, or a severe adhesion dysfunction in a cell surface adhesion molecule, wherein the processor provides a real-time quantitative measurement of a dynamic range of cell or platelet function; and
based on the dynamic range of cell or platelet function from the cell or platelet sample determining the course of treatment for the subject.

19. The method of claim 18, wherein the cell surface adhesion molecule is von Willebrand Factor, an ICAM, a VCAM, a Lectin, an Integrin, a collagen, a fibrinogen, a subendothelial membrane protein, glycoprotein Ib(β), glycoproteinIb(α), glycoprotein Ib-IX-V complex, glycoprotein IIbIIIa, glyprotein VI, major histocompatibility complex, integrin α2 β1, or immunoglobin.

20. The method of claim 18, wherein the cell surface adhesion molecule is von Willebrand Factor and the mutations are point mutations in the A1, A2, A3, B, or C domains.

21. The method of claim 18, wherein the surface is coated with a mutant human von Willebrand factor.

22. The method of claim 18, wherein the cells or platelets are obtained from a suspect suspected of having von Willebrand disease, platelet type von Willebrand disease, acquired von Willebrand syndrome, hypertrophic cardiomyopathy, Bernard-Soulier syndrome, Glanzmann's thrombasthenia thrombocytopenia, or various autoimmune coagulation disorders.

23. The method of claim 18, wherein the cells or platelets are obtained from a suspect suspected of having coagulopathies associated with left ventricular assist device implantation.

24. The method of claim 18, wherein the surface is in a multifluidic chamber.

25. The method of claim 18, wherein the surface is coated with a truncated human von Willebrand factor fusion protein that comprises a human von Willebrand factor platelet adhesion domain and a recombinant binding domain.

26. The method of claim 18, wherein the processor calculates the dynamic range of cell or platelet adhesion molecule to distinguish between two or more low adhesion cell or platelet binding dysfunctions that is statistically significant.

27. The method of claim 18, wherein the method distinguishes between different low adhesion cell or platelet binding dysfunctions that is statistically significant.

28. The method of claim 18, wherein the cells are selected from T cells, B cells, macrophages, neutrophils, basophils, or eosinophils.

29. The method of claim 18, wherein the agent comprises an ethylene glycol polymer bound to the surface and a bivalent cation to provide a consistent binding surface for a His-tag or equivalent bivalent cation binding peptide or polypeptide that is removably attached with high affinity to the bivalent cation.

30. The method of claim 18, wherein between 100 to 1000, 1000 to 10,000, 10,000 to 15,000, 25,000 to 50,000, 50 to 75,000, 75,000 to 100,000, 100 to 100,000, 1000 to 90,000, 20,000 to 80,000, 30,000 to 70,000, 40,000 to 60,000 or more than 100,000 cells or platelets are imaged and processed to determine the median and the mean roll time of cells or platelets is determined to calculate the level of adhesion of the cells or platelets to the agent on the surface.

31. The method of claim 18, wherein the dynamic range is in the low adhesion level.

32. The method of claim 18, wherein the dynamic range is in the low adhesion level and provides differentiation between various low adhesion point mutants in von Willebrand disease.

33. The method of claim 18, wherein the processor calculates the adhesion and determines if the von Willebrand Factor is natively structured, nativelike 2M, nativelike 2B, molten globule 2M or molten globule 2B.

34. The method of claim 18, wherein the apparatus distinguished between a native von Willebrand Factor A1 domain and one or more of the following mutations: G1324S (2M); A1437T (2M); R1308L (2B); R1341Q (2B); R1306Q (2B); I1372S (2B); I1309V (2B); F1369I (2M); E1359K (2M); I1425F (2M); S1285F (2M); R1374H (2M); H1268D (2B); or V1316M.

35. The method of claim 18, wherein the method further comprises the steps of:
obtaining cell or platelet position data at a first time for many translocation events, wherein each event represents the 2-dimensional movement of a single cell or platelet moving over time;
obtaining additional, subsequent position data for each of the cells or platelets at a second or subsequent point in time;
calculating distance trajectories from coordinate data as a function of time and numerically differentiated using a Savitzky-Golay algorithm to obtain instantaneous velocities and accelerations as a function of time for every moving cell or platelet;
reporting the properties are instantaneous velocities and accelerations as a function of time for each X and Y component directions;
calculating pause times from the amount of time a cell or platelet is motionless, a distribution of the pause times, velocities and accelerations; and
plotting the pause times and shear rates calculated from the pause times, velocities and accelerations to determine the level of adhesion of the cells or platelets to the surface.

36. A method of evaluating a candidate drug for changing cell or platelet adhesion comprising:
(a) measuring the transit of a cell or platelet sample on a rheological shear flow surface under shear flow from a patient having a platelet adhesion dysfunction;
(b) calculating both a pause time and a roll time of cells or platelets of the cell or platelet sample to determine a median and a mean pause time and a median and a mean roll time of cell or platelets;
(c) comparing the median and mean pause times and the median and mean roll times of the cell or platelets in the cell or platelet sample to a sample obtained from a subject that does not have a cell or platelet adhesion dysfunction to provide a real-time quantitative measurement of a dynamic range of cell or platelet function;
(d) exposing the platelets from the cell or platelet sample to a candidate drug;
(e) repeating steps (a) to (c) after the exposing the cells or platelets to the candidate drug; and
(f) determining if the candidate drug changes the median and mean pause times and the median and mean roll times of the cells or platelets that is statistically significant as compared to any reduction in the cells or platelets from the subject that does not have a cell or platelet adhesion dysfunction, wherein a statistically significant reduction indicates that the candidate drug is useful in changing cell or platelet adhesion.

37. A method of performing a clinical trial to evaluate a candidate drug believed to be useful in treating a bleeding diatheses, the method comprising:
(a) measuring the transit of a cell or platelet sample on a rheological shear flow surface under shear flow from a set of patients;
(b) calculating both a pause time and a roll time of cells or platelets of the cell or platelet sample to determine a median and a mean pause time and a median and a mean roll time of cells or platelets;
(c) comparing the median and mean pause times and the median and mean roll times of the cells or platelets in the platelet sample to provide a real-time quantitative measurement of a dynamic range of platelet function;
(d) administering a candidate drug to a first subset of the patients, and a placebo to a second subset of the patients;
(e) repeating steps (a) to (c) after the administration of the candidate drug or the placebo; and
(f) determining if the candidate drug changes the median and mean pause times and the median and mean roll times of the cells or platelets that is statistically significant as compared to any reduction occurring in the second subset of patients, wherein a statistically significant reduction indicates that the candidate drug is useful in treating the bleeding diatheses.

38. A method of using a real-time quantitative measurement of a dynamic range of cell or platelet function to select a treatment comprising:
obtaining a cell or platelet sample from a subject suspected of having a dysfunction in platelet adhesion;
flowing the cell or platelet sample over a rheological shear flow surface, wherein the surface is coated with an agent that provides cell or platelet binding;
measuring the transit of the cell or platelet sample on the surface under shear flow;
calculating both a pause time and a roll time of the cells or platelets obtained from a subject suspected of having a cell or platelet dysfunction, wherein the processor determines a median and a mean pause time and a median and a mean roll time of cells or platelets and compares them to platelets having at least one of: no cell or platelet adhesion dysfunction, a mild adhesion dysfunction, or a severe adhesion dysfunction in a cell surface adhesion molecule, wherein the processor provides a real-time quantitative measurement of a dynamic range of cell or platelet function;
plotting the pause time and roll times for the cells or platelets, wherein the plots distinguish between various low adhesion diseases or conditions; and
based on the dynamic range of cell or platelet function from the cell or platelet sample determining the course of treatment for the subject.
